SEQUENCE LISTING (33 sequences)

SEQ ID NO: 1 - protein, 14507 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 2 - protein, 115 aa, synthetic construct
  REGION 1..115: note = Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSS

SEQ ID NO: 3 - protein, 108 aa, synthetic construct
  REGION 1..108: note = Description of Artificial Sequence: Synthetic polypeptide
DIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKR

SEQ ID NO: 4 - protein, 8 aa, synthetic construct
  REGION 1..8: note = Description of Artificial Sequence: Synthetic peptide
GGSFSGYY

SEQ ID NO: 5 - protein, 7 aa, synthetic construct
  REGION 1..7: note = Description of Artificial Sequence: Synthetic peptide
INHSGST

SEQ ID NO: 6 - protein, 9 aa, synthetic construct
  REGION 1..9: note = Description of Artificial Sequence: Synthetic peptide
ARQSYITDS

SEQ ID NO: 7 - protein, 6 aa, synthetic construct
  REGION 1..6: note = Description of Artificial Sequence: Synthetic peptide
QDVSKW

SEQ ID NO: 8 - skipped

SEQ ID NO: 9 - protein, 9 aa, synthetic construct
  REGION 1..9: note = Description of Artificial Sequence: Synthetic peptide
QQANSFPWT

SEQ ID NO: 10 - protein, 120 aa, synthetic construct
  REGION 1..120: note = Description of Artificial Sequence: Synthetic polypeptide
QVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSS

SEQ ID NO: 11 - protein, 112 aa, synthetic construct
  REGION 1..112: note = Description of Artificial Sequence: Synthetic polypeptide
NFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLG

SEQ ID NO: 12 - protein, 8 aa, synthetic construct
  REGION 1..8: note = Description of Artificial Sequence: Synthetic peptide
GGSFSGYY

SEQ ID NO: 13 - protein, 7 aa, synthetic construct
  REGION 1..7: note = Description of Artificial Sequence: Synthetic peptide
INHSGST

SEQ ID NO: 14 - protein, 14 aa, synthetic construct
  REGION 1..14: note = Description of Artificial Sequence: Synthetic peptide
ARWSPFSYKQMYDY

SEQ ID NO: 15 - protein, 8 aa, synthetic construct
  REGION 1..8: note = Description of Artificial Sequence: Synthetic peptide
RGSIASAY

SEQ ID NO: 16 - skipped

SEQ ID NO: 17 - protein, 10 aa, synthetic construct
  REGION 1..10: note = Description of Artificial Sequence: Synthetic peptide
QSYDDNDHVI

SEQ ID NO: 18 - protein, 20 aa, synthetic construct
  REGION 1..20: note = Description of Artificial Sequence: Synthetic peptide
METDTLLLWVLLLWVPGSTG

SEQ ID NO: 19 - protein, 244 aa, synthetic construct
  REGION 1..244: note = Description of Artificial Sequence: Synthetic polypeptide
DIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKRSRGGGGSGGGGSGGGGSLEMAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSS

SEQ ID NO: 20 - protein, 253 aa, synthetic construct
  REGION 1..253: note = Description of Artificial Sequence: Synthetic polypeptide
NFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLGSRGGGGSGGGGSGGGGSLEMAQVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSS

SEQ ID NO: 21 - protein, 243 aa, synthetic construct
  REGION 1..243: note = Description of Artificial Sequence: Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 22 - protein, 520 aa, synthetic construct
  REGION 1..520: note = Description of Artificial Sequence: Synthetic polypeptide
METDTLLLWVLLLWVPGSTGDIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKRSRGGGGSGGGGSGGGGSLEMAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 23 - protein, 529 aa, synthetic construct
  REGION 1..529: note = Description of Artificial Sequence: Synthetic polypeptide
METDTLLLWVLLLWVPGSTGNFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLGSRGGGGSGGGGSGGGGSLEMAQVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 24 - protein, 344 aa, synthetic construct
  REGION 1..344: note = Description of Artificial Sequence: Synthetic polypeptide
WELSQLTHGVTQLGFYVLDRDSLFINGYAPQNLSIRGEYQINFHIVNQNLSNPDPTSSEYITLLRDIQDKVTTLYKGSQLHDTFRFCLVTNLTMDSVLVTVKALFSSNLDPSLVEQVFLDKTLNASFHQLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 25 - protein, 114 aa, synthetic construct
  REGION 1..114: note = Description of Artificial Sequence: Synthetic polypeptide
NFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 26 - protein, 80 aa, synthetic construct
  REGION 1..80: note = Description of Artificial Sequence: Synthetic polypeptide
NFSPLARRVDRVAIYEEFLRMDLPFWAVILIGLAGLLGLITCLICGVLVTTRRRKKEGEYNVQQQCPGYYQSHLDLEDLQ

SEQ ID NO: 27 - protein, 86 aa, synthetic construct
  REGION 1..86: note = Description of Artificial Sequence: Synthetic polypeptide
NFSPLARRVDRVAIYEEFLRMTRNGTQLQNFTLDRSSVLVDGYSPNRNEPLTGNSDLPFWAVILIGLAGLLGLITCLICGDLEDLQ

SEQ ID NO: 28 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 105 aa, synthetic construct
  REGION 1..105: note = Description of Artificial Sequence: Synthetic polypeptide
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 31 - protein, 114 aa, synthetic construct
  REGION 1..114: note = Description of Artificial Sequence: Synthetic polypeptide
NFSPLARRVDRVAIYEEFLRMTRNGTQLQAFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 32 - protein, 14447 aa, Homo sapiens
DKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 33 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2025/0361321 A1
(43) Pub. Date: Nov. 27, 2025

(54) ANTIBODIES TO MUCIN-16 AND METHODS OF USE THEREOF

(71) Applicants: Memorial Sloan Kettering Cancer Center, New York, NY (US); Eureka Therapeutics, Inc., Emeryville, CA (US)

(72) Inventors: David SPRIGGS, New York, NY (US); Javier MORALES, Emeryville, CA (US); Yoko NAKANO, Emeryville, CA (US); Hong LIU, Emeryville, CA (US)

(21) Appl. No.: 18/983,001

(22) Filed: Dec. 16, 2024

Related U.S. Application Data
(63) Continuation of application No. 17/292,749, filed on May 11, 2021, now Pat. No. 12,168,696.
(60) Provisional application No. 62/768,730, filed on Nov. 16, 2018.

(51) Int. Cl. C07K 16/30; A61K 39/00; A61P 35/00

(52) U.S. Cl. CPC C07K 16/3092; A61P 35/00; A61K 2039/505; C07K 2317/21; C07K 2317/31; C07K 2317/34; C07K 2317/622; C07K 2317/73; C07K 2317/76

(57) ABSTRACT

Provided herein are compositions, methods, and uses involving anti-Mucin-16 (MUC16) agents that immunospecifically bind an epitope of Mucin-16 (MUC16). Also provided herein are uses and methods for managing, treating, or preventing disorders, such as cancer and diseases associated with positive MUC16 expression.

### CROSS-REFERENCE TO RELATED PATENT APPLICATIONS

This application is a continuation of U.S. application Ser. No. 17/292,749, filed May 11, 2021, which is the U.S. National Stage Application of International Application No. PCT/US2019/061503, filed Nov. 14, 2019, which claims the benefit of and priority to U.S. Provisional Appl. No. 62/768,730, filed Nov. 16, 2018, the disclosure of each of which is incorporated by reference herein in its entirety.

### STATEMENT OF GOVERNMENT SUPPORT

This invention was made with government support under CA190174 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Apr. 22, 2025, is named 115872-3149_SL.xml and is 9,214,324,493 bytes in size. The 115872-3149_SL.xml ST.26 format was converted from the 115872-0494_SL.txt sequence listing prepared Dec. 23, 2019, on Apr. 22, 2025.

### BACKGROUND OF THE INVENTION

Mucins are important biomolecules for cellular homeostasis and protection of epithelial surfaces. Changes in expression of mucins in cancers, such as ovarian cancer, are useful as a biomarker for diagnosis, prognosis and treatment (Singh A P, et al., Lancet Oncol 2008: 9(11): 1076-85). MUC16 is a mucin that is over expressed on most ovarian carcinoma cells and is an established surrogate serum marker (CA-125) for the detection and progression of ovarian cancers (Badgwell D, et al., Dis Markers 23(5-6):397410 (2007); Bast R C, Jr, et al., Int J Gynecol Cancer 15 Suppl 3:274-81 (2005); Fritsche H A, et al., Clin Chem 44 (7): 1379-80 (1998); and Krivak T C et al., Gynecol Oncol 115 (1): 81-5 (2009)).

MUC16 is a highly glycosylated mucin composed of a large extracellular domain (CA-125), which is cleaved and released, and a retained domain (MUC-CD) (FIG. 1). MUC-CD comprises a non-repeating extracellular domain (MUC16 ectodomain) proximal to a cleavage site, a transmembrane domain, and a cytoplasmic tail with potential phosphorylation sites. Distal to the cleavage site, the released extracellular domain (CA-125) contains 16-20 tandem repeats of 156 amino acids, each with many potential glycosylation sites (O'Brien T J, et al., Tumor Biol 22(6):348-66 (2001)). Since the MUC16 antigen is otherwise expressed only at low levels in normal tissues of the uterus, endometrium, fallopian tubes, ovaries, and serosa of the abdominal and thoracic cavities, MUC16 is a potentially attractive target for immune-based therapies, including the targeting and treatment of cancer.

A significant portion of the extracellular domain of MUC16 is cleaved and secreted (i.e., CA-125), which limits the utility of this portion of MUC16 to be used as a target antigen on ovarian carcinomas. Many reported MUC16 monoclonal antibodies bind to epitopes present on the large secreted CA-125 fraction of the glycoprotein, and not to the retained MUC16 ectodomain (Bellone S Am. J Obstet Gynecol 200(1):75 e1-10 (2009), Berek J S. Expert Opin Biol Ther. 4(7): 1159-65 (2004); O'Brien T J, et al., Int J Biol Markers 13(4): 188-95 (1998)). Thus, the generation of new antibodies to the region of MUC16 that is not shed are needed for diagnostic and therapeutic purposes.

### SUMMARY OF THE INVENTION

Provided herein are compositions, methods, and uses of anti-Mucin 16 (MUC16) constructions that comprise antibody moieties that immunospecifically bind to Mucin 16 (MUC16), and modulate expression and/or activity of MUC16 for managing or treating MUC16-mediated disorders, such as cancer.

Provided herein, in certain embodiments, are anti-mucin 16 (MUC16) constructs comprising an antibody moiety that immunospecifically recognizes a mucin 16 (MUC16) polypeptide, wherein the antibody moiety comprises (a) (i) a variable heavy (VH) chain comprising a heavy chain complementarity determining region (HC-CDR) 1, an HC-CDR2, and an HC-CDR3 of the heavy chain variable domain of SEQ ID NO: 2; and (ii) a variable light (VL) chain comprising a light chain complementarity determining region (LC-CDR) 1, an LC-CDR2, and an LC-CDR3 of the light chain variable domain of SEQ ID NO: 3; or (b) (i) a variable heavy (VH) chain comprising a heavy chain complementarity determining region (HC-CDR) 1, an HC-CDR2, and an HC-CDR3 of the heavy chain variable domain of SEQ ID NO: 10; and (ii) a variable light (VL) chain comprising a light chain complementarity determining region (LC-CDR) 1, an LC-CDR2, and an LC-CDR3 of the light chain variable domain of SEQ ID NO: 11. In some embodiments, the antibody moiety immunospecifically recognizes a human MUC16. In some embodiments, the MUC16 is glycosylated. In some embodiments, the MUC16 is N-glycosylated at Asn1800 or Asn1806.

In some embodiments, the antibody moiety of the anti-mucin 16 (MUC16) constructs provided herein comprises (a) (i) a variable heavy (VH) chain comprising a heavy chain complementarity determining region (HC-CDR) 1 comprising the amino acid sequence of SEQ ID NO: 4; a HC-CDR2 comprising the amino acid sequence of SEQ ID NO: 5; and a HC-CDR3 comprising the amino acid sequence of SEQ ID NO: 6; and (ii) a variable light (VL) chain comprising: a light chain complementarity determining region (LC-CDR) 1 comprising the amino acid sequence of SEQ ID NO: 7; a LC-CDR2 comprising the amino acid sequence of SEQ ID NO: 8; and a LC-CDR3 comprising the amino acid sequence of SEQ ID NO: 9; or (b) (i) a variable heavy (VH) chain comprising a HC-CDR1 comprising the amino acid sequence of SEQ ID NO: 12; a HC-CDR2 comprising the amino acid sequence of SEQ ID NO: 13; and a HC-CDR3 comprising the amino acid sequence of SEQ ID NO: 14; and (ii) a variable light (VL) chain comprising: a LC-CDR1 comprising the amino acid sequence of SEQ ID NO: 15; a LC-CDR2 comprising the amino acid sequence of SEQ ID NO: 16; and a LC-CDR3 comprising the amino acid sequence of SEQ ID NO: 17.

In some embodiments, the antibody moiety of the anti-mucin 16 (MUC16) constructs provided herein immunospecifically binds to the ectodomain of MUC16. In some embodiments, the antibody moiety is a full-length antibody, a Fab, a Fab′, a F(ab′)2, an Fv, or a single chain Fv (scFv). In some embodiments, the VH chain and the VL chain are human VH chain and VL chain. In some embodiments, the antibody moiety is a monoclonal antibody. In some embodiments, the antibody moiety immunospecifically binds to a MUC16 c114 polypeptide comprising the amino acid sequence of SEQ ID NO: 25.

In some embodiments, the anti-MUC16 constructs provided herein inhibit in vitro invasion of a tumor cell that expresses MUC16 in a Matrigel invasion assay. In some embodiments, the tumor cell is an ovarian tumor cell.

In some embodiments, the antibody moiety of the anti-mucin 16 (MUC16) constructs provided herein comprises a VH comprising the amino acid sequence of SEQ ID NO: 2. In some embodiments, the antibody moiety comprises a VL comprising the amino acid sequence of SEQ ID NO: 3. In some embodiments, the antibody moiety comprises a VH comprising the amino acid sequence of SEQ ID NO: 10. In some embodiments, the antibody moiety comprises a VL comprising the amino acid sequence of SEQ ID NO: 11. In some embodiments, the antibody moiety comprises a VH comprising the amino acid sequence of SEQ ID NO: 2 and a VL comprising the amino acid sequence of SEQ ID NO: 3. In some embodiments, the antibody moiety comprises a VH comprising the amino acid sequence of SEQ ID NO: 10 and a VL comprising the amino acid sequence of SEQ ID NO: 11. In some embodiments, the antibody moiety comprises human-derived heavy and light chain constant regions. In some embodiments, the heavy chain constant region has an isotype selected from the group consisting of gamma 1, gamma 2, gamma 3, and gamma 4. In some embodiments, the light chain constant region has an isotype selected from the group consisting of kappa and lambda. In some embodiments, the antibody moiety is an immunoglobulin comprising two identical heavy chains and two identical light chains. In some embodiments, the immunoglobulin is an IgG.

In some embodiments, the anti-MUC16 construct provided herein is monospecific. In some embodiments, the anti-MUC16 construct provided herein is multispecific. In some embodiments, the anti-MUC16 construct provided herein is bispecific. In some embodiments, the anti-MUC16 construct provided herein is a tandem scFv, a diabody (Db), a single chain diabody (scDb), a dual-affinity retargeting (DART) antibody, a F(ab′)2, a dual variable domain (DVD) antibody, a knob-into-hole (KiH) antibody, a dock and lock (DNL) antibody, a chemically cross-linked antibody, a beteromultimeric antibody, or a heteroconjugate antibody. In some embodiments, the anti-MUC16 construct provided herein is a tandem scFv comprising two scFvs linked by a peptide linker. In some embodiments, the antibody moiety that immunospecifically recognizes MUC16 is a first antibody moiety, and wherein the anti-MUC16 construct further comprises a second antibody moiety that immunospecifically recognizes a second antigen. In some embodiments, the second antigen is an antigen on the surface of a T cell. In some embodiments, the second antigen is a CD3. In some embodiments, the second antigen is selected from the group consisting of CD3γ, CD3δ, CD3ε, and CD3ζ. In some embodiments, the second antigen is CD3ε.

In some embodiments, the anti-MUC16 construct provided herein is a chimeric antigen receptor (CAR). In some embodiments, the CAR comprises a co-stimulatory domain. In some embodiments, the CAR comprises a CD3 zeta (ζ) chain cytoplasmic signaling domain.

In some embodiments, the anti-MUC16 construct provided herein is further conjugated to a peptide agent, a detection agent, an imaging agent, a therapeutic agent, or a cytotoxic agent.

Also provided herein, in certain embodiments, are polypeptides comprising an amino acid sequence of one or more of SEQ ID NOs: 2-17 or an amino acid of an anti-MUC16 construct provided herein.

Also provided herein, in certain embodiments, are polynucleotides comprising a nucleic acid sequence encoding one or more polypeptides comprising an amino acid sequence of one or more of SEQ ID NOs: 2-17 or an amino acid of an anti-MUC16 construct provided herein. Provided herein, in certain embodiments, are vectors comprising the polynucleotide provided herein operably linked to a promoter.

Also provided herein, in certain embodiments, are cells comprising the anti-MUC16 construct provided herein, a polypeptide provided herein, a polynucleotide provided herein, or a vector provided herein. In some embodiments, the cell is a mammalian cell. In some embodiments, the cell is an immune cell. In some embodiments, the cell is a lymphocyte. In some embodiments, the cell is a T cell or a B cell.

Also provided herein, in certain embodiments, are pharmaceutical compositions comprising: a therapeutically effective amount of the anti-MUC16 construct provided herein, a polypeptide provided herein, polynucleotide provided herein, or a vector provided herein; and a pharmaceutically acceptable carrier.

Also provided herein, in certain embodiments, are methods of treating a MUC16-associated disease or disorder in a patient in need thereof, comprising administering to said patient a pharmaceutical composition comprising a therapeutically effective amount of the anti-MUC16 construct provided herein, a polypeptide provided herein, polynucleotide provided herein, or a vector provided herein. In some embodiments, the MUC16-associated disease or disorder is a cancer. In some embodiments, the cancer is a cancer of the ovary, lung, pancreas, breast, uterine, fallopian tube, or primary peritoneum. In some embodiments, the cancer is a metastatic cancer. In some embodiments, the pharmaceutical composition inhibits metastasis in the patient. In some embodiments, the patient is a human patient.

Also provided herein, in certain embodiments, are methods for producing an effector cell, comprising genetically modifying a cell with one or more nucleic acids encoding the anti-MUC16 construct provided herein.

Also provided herein, in certain embodiments, are methods of comprising introducing one or more nucleic acids encoding the anti-MUC16 construct provided herein into one or more primary cells isolated from a patient and administering cells comprising the one or more nucleic acids to the patient. In some embodiments, the method further comprises expanding the cells prior to administering the cells to the patient. In some embodiments, the primary cells are lymphocytes. In some embodiments, the primary cells are T cells.

In some embodiments, the methods of treatment provided herein further comprises administering a therapeutically effective amount of an additional therapeutic agent to the patient. In some embodiments, the therapeutic agent is an anti-cancer agent. In some embodiments, the therapeutic agent is a chemotherapeutic agent.

Also provided herein, in certain embodiments, are methods of detecting MUC16 in a sample, comprising: (a) contacting the sample with the anti-MUC16 construct provided herein; and (b) detecting the binding, directly or indirectly, between the anti-MUC16 construct and MUC16 that is present in the sample. In some embodiments, the anti-MUC16 construct is conjugated to a detectable label. In some embodiments, the detectable label is a chromogenic, enzymatic, radioisotopic, isotopic, fluorescent, toxic, chemiluminescent, nuclear magnetic resonance contrast agent. In some embodiments, the binding between the anti-MUC16 construct and any MUC16 in the sample is detected directly by detecting the detectable label. In some embodiments, the binding between the anti-MUC16 construct and any MUC16 in the sample is detected indirectly using a secondary antibody.

Also provided herein, in certain embodiments, are methods of diagnosing an individual suspected of having a MUC16-associated disease or disorder, comprising a) administering an effective amount of the anti-MUC16 construct provided herein to the individual; and b) determining the level of the binding, directly or indirectly, between the anti-MUC16 construct and any MUC16 in the individual, wherein a level of the binding above a threshold level indicates that the individual has the MUC16-associated disease or disorder. In some embodiments, the anti-MUC16 construct is conjugated to a detectable label. In some embodiments, the detectable label is a chromogenic, enzymatic, radioisotopic, isotopic, fluorescent, toxic, chemiluminescent, nuclear magnetic resonance contrast agent. In some embodiments, the binding between the anti-MUC16 construct and any MUC16 in the sample is detected directly by detecting the detectable label. In some embodiments, the binding between the anti-MUC16 construct and any MUC16 in the sample is detected indirectly using a secondary antibody.

A method of diagnosing an individual suspected of having a MUC16-associated disease or disorder, comprising a) contacting a sample comprising cells derived from the individual with the anti-MUC16 construct provided herein; and b) determining the number of cells in the sample bound to the anti-MUC16 construct, wherein a value for the number of cells bound to the anti-MUC16 construct above a threshold level indicates that the individual has the MUC16-associated disease or disorder. In some embodiments, the anti-MUC16 construct is conjugated to a detectable label. In some embodiments, the detectable label is a chromogenic, enzymatic, radioisotopie, isotopic, fluorescent, toxic, chemiluminescent, nuclear magnetic resonance contrast agent. In some embodiments, the binding between the anti-MUC16 construct and any MUC16 in the sample is detected directly by detecting the detectable label. In some embodiments, the binding between the anti-MUC16 construct and any MUC16 in the sample is detected indirectly using a secondary antibody.

Also provided herein, in certain embodiments, are methods of generating an anti-MUC16 construct that immunospecifically binds to a human MUC16 polypeptide, comprising selecting a human scFv specific for human MUC16 from a human scFv antibody phage display library. In some embodiments, selecting a human scFv specific for human MUC16 comprises contacting the human scFv antibody phage display library with a cell that expresses a recombinant MUC16 polypeptide. In some embodiments, the recombinant MUC16 polypeptide comprises the sequence of SEQ ID NO: 25.

Also provided herein, in certain embodiments, are uses of anti-MUC16 constructs, anti-MUC16 polypeptides, polynucleotides encoding anti-MUC16 constructs or anti-MUC16 polypeptides, vectors comprising the polynucleotides, or cells comprising any the polypeptides and polynucleotides thereof provided herein for the treatment of a disease or disorder associated with positive MUC16 expression. In some embodiments, the disease or disorder associated with positive MUC16 expression is a cancer.

Also provided herein, in certain embodiments, are uses of the anti-MUC16 constructs, anti-MUC16 polypeptides, polynucleotides encoding anti-MUC16 constructs or anti-MUC16 polypeptides, vectors comprising the polynucleotides, or cells comprising any the polypeptides and polynucleotides thereof provided herein in the manufacture of a medicament for the treatment of a disease or disorder associated with positive MUC16 expression. In some embodiments, the disease or disorder associated with positive MUC16 expression is a cancer.

Also provided herein, in certain embodiments, are uses of anti-MUC16 constructs, anti-MUC16 polypeptides, polynucleotides encoding anti-MUC16 constructs or anti-MUC16 polypeptides, vectors comprising the polynucleotides, or cells comprising any the polypeptides and polynucleotides thereof provided herein for the diagnosis of a disease or disorder associated with positive MUC16 expression. In some embodiments, the disease or disorder associated with positive MUC16 expression is a cancer.

### BRIEF DESCRIPTION OF THE DRAWINGS

FIGS. 1A-1B show a schematic illustration of the structure of MUC16 shows and a schematic and amino acid sequence of the truncated form of MUC16, respectively, called MUC16 c114 (SEQ ID NO: 25), which includes the 58 amino acid ectodomain, the 25 amino acid transmembrane domain, and the 31 amino acid cytoplasmic tail. Numbering in figure is based on original publication identifying Muc16, Yin and Lloyd (2001) J Biol Chem 276; 27371-27375

FIG. 2 shows an amino acid alignment between wildtype MUC16-C114 (SEQ ID NO: 25) and the N30 mutant MUC16-C114 (SEQ ID NO: 31) ectodomains.

FIG. 3 illustrates fluorescence activated cell sorting (FACS) analysis of GFP expression in HEK293 stable cell lines that express wild-type MUC16-C114 (HEK293-MUC16WT) or N30 mutant MUC16-C114 (HEK293-MUC16mut). Control HEK293 cells are shown for comparison.

FIG. 4 illustrates results of a FACS analysis wild-type MUC16-C114 (HEK293-MUC16WT) or N30 mutant MUC16-C114 (HEK293-MUC16mut) cells incubated with a negative phage control and a no phage control for all three cell lines.

FIG. 5 illustrates exemplary results of a FACS analysis wild-type MUC16-C114 (HEK293-MUC16WT) or N30 mutant MUC16-C114 (HEK293-MUC16mut) cells incubated with two exemplary antibody phage clones, clone 8 and clone 12. Antibody clones that bound to wild-type MUC16-C114 and not N30 mutant MUC16-C114 were selected for sequencing and further development.

FIG. 6 illustrates the binding of the clone 8 bispecific antibody (BsAb), comprising the anti-MUC16 scFv at the N-terminal end and an anti-human CD3e scFv of a mouse monoclonal antibody at the C-terminal end, to a MUCH6+ OVCAR3 cell line, but not a control MUC16 SKOV3 cell line.

FIG. 7 illustrates exemplary results of a cell cytotoxicity assay using selected anti-MUC16 BsAbs, including anti-MUC16 clone 8 BsAb and anti-MUC16 clone 12 BsAb, incubated with a MUC16+ OVCAR3 cell line compared to a MUC16⁻ SKOV3 cell line. The clone 8 and clone 12 BsAbs were able to induce target specific cell lysis of a MUC16⁺ OVCAR3 cell line as compared to a MUC16-SKOV3 cell line.

FIG. 8 illustrates exemplary results of a cell cytotoxicity assay using selected anti-MUC16 BsAbs, including anti-MUC16 clone 8 BsAb and anti-MUC16 clone 12 BsAb, incubated with a MUC16+ OVCAR3 cell line, a MUC16′ SKOV8 cell line, and a MUC16′ OVCA432 cell line compared to a MUC16 SKOV3 cell line. The anti-MUC16 clone 8 BsAb was able to induce target specific cell lysis of the MUC16+ OVCAR3, SKOV8, and OVCA432 cell lines. The anti-MUC16 clone 12 BsAb also induced cell lysis of the MUC16+, though to a lesser extent.

FIGS. 9A-9D illustrate an exemplary experimental schema of an ovarian xenograft study using injections of SKOV3-MUC-CD modified cells that express MUC16 to establish tumors and injections of the anti-MUC16 clone & BsAb for treatment, an exemplary visualization data show establishment and treatment of the tumors, an exemplary survival curve data for the xenograft experiment, and an exemplary data showing induction of cytokines IL-2 and IFN-γ following treatment of tumor bearing mice with anti-MUC16 clone 8 BsAb respectively.

### DETAILED DESCRIPTION OF THE INVENTION

The present application in one aspect provides anti-MUC16 antibody agents, such as anti-MUC16 constructs that comprise an antibody moiety that specifically recognizes an epitope of MUC16, such as an epitope of the retained extracellular domain of MUC16 (MUC16 ectodomain).

Using phage display technology, scFvs that are specific for the retained extracellular domain of human MUC16 were identified. Flow cytometry assays demonstrated that these antibodies recognize MUC16-expressing cancer cell lines. The present application thus provides anti-MUC16 antibody agents, such as anti-MUC16 constructs that comprise an antibody moiety that immunospecifically binds MUC16, The anti-MUC16 antibody agents include, for example, anti-MUC16 antibodies, e.g., full-length anti-MUC16 antibodies and antigen-binding fragments thereof, anti-MUC16 scFvs, anti-MUC16 antibody fusion proteins (e.g., anti-MUC16 Fc fusion proteins and chimeric antigen receptors (CAR)), multi-specific antibodies, e.g., bispecific antibodies, and anti-MUC16 antibody conjugates (i.e., anti-MUC16 immunoconjugates) thereof.

In another aspect, provided are nucleic acids encoding the anti-MUC16 antibody agents, such as anti-MUC16 antibodies, e.g., full-length anti-MUC16 antibodies and antigen-binding fragments thereof, anti-MUC16 seFvs, anti-MUC16 antibody fusion proteins (e.g., anti-MUC16 Fc fusion proteins and chimeric antigen receptors (CAR)), multi-specific antibodies, e.g., bispecific antibodies, and anti-MUC16 antibody conjugates (i.e., anti-MUC16 immunoconjugates) thereof.

In another aspect, provided are compositions, such as pharmaceutical compositions, comprising an anti-MUC16 antibody agent, such as full-length anti-MUC16 antibodies and antigen-binding fragments thereof, anti-MUC16 scFvs, anti-MUC16 antibody fusion proteins (e.g., anti-MUC16 Fc fusion proteins and chimeric antigen receptors (CAR)), multi-specific antibodies, e.g., bispecific antibodies, and anti-MUC16 antibody conjugates (i.e., anti-MUC16 immunoconjugates) thereof.

Also provided are methods of making and using the anti-MUC16 antibody agents and antibodies, such as for treating cancer, as well as kits and articles of manufacture useful for such methods.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al., (eds.). Springer Verlag (19) 1); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

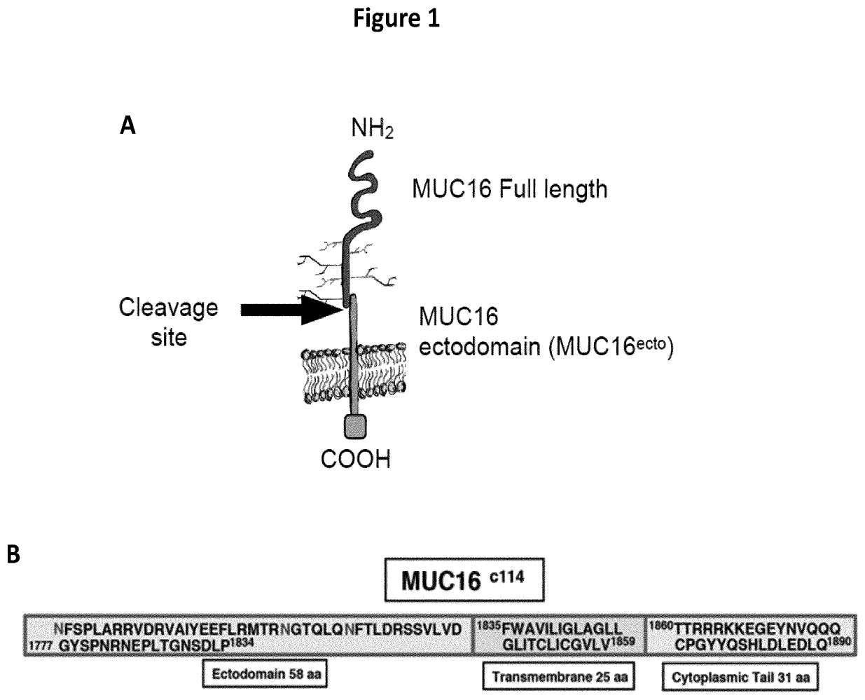

As used herein, the term “MUC 16” or “MUC 16 polypeptide” or “MUC 16 peptide” refers to the MUC 16 tethered mucin protein as described in Yin B W and Lloyd K O, 2001, J Biol Chem. 276 (29): 27371-5. GenBank™ accession number NP_078966.2 (SEQ ID NO: 1) provides an exemplary human MUC 16 nucleic acid sequence. GenBank™ accession number NP 078966.2 (SEQ ID NO: 1) provides an exemplary human MUC16 amino acid sequence. Native MUC 16 comprises an intracellular domain, a transmembrane domain, an ectodomain proximal to the putative cleavage site, and a large, heavily glycosylated region of 12-20 repeats, each 156 amino acids long (FIG. 1A). “Immature” MUC16 refers to SEQ ID NO: 1, which comprises the MUC16 signal sequence (amino acid residues 1-60 of SEQ ID NO: 1). “Mature MUC 16” refers to native MUC 16 as expressed on the cell surface, i.e., where the signal sequence has been removed by cellular processing, for example, SEQ ID NO: 32, where the first 60 amino acid residues of SEQ ID NO: 1 have been removed (i.e., SEQ ID NO: 1 is the “immature” form of MUC16).

The polypeptide represented by the amino acid sequence of SEQ ID NO: 25 is referred to herein as MUC16 C114 and consists of the C-terminal 114 amino acid residues of mature MUC16 (SEQ ID NO: 32 being the sequence of mature MUC16). MUC16 C114 comprises a 58 amino acid ectodomain, a 25 amino acid transmembrane domain and a 31 amino acid cytoplasmic tail (FIG. 1B). MUC16c114 is capable of being N-glycosylated at the asparagine amino acid residues at positions 1, 24, and 30 of SEQ ID NO: 25 (also referred to as amino acid positions Asnl777, Asn1800, and Asn1806 according the original MUC16 publication Yin B W and Lloyd K O, 2001. J Biol Chem. 276(29):27371-5).

As used herein, the singular forms “a”, “an” and “the” are intended to include the plural forms as well, unless the context clearly indicates otherwise.

As used herein, the terms “about” when used to modify a numeric value or numeric range, indicate that deviations of 5% to 10% above and 5% to 10% below the value or range remain within the intended meaning of the recited value or range.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form and a second plurality are in the L form.

Amino acids are referred to herein by either their commonly known three letter symbols or by the one-letter symbols recommended by the TUPAC-IUB Biochemical Nomenclature Commission. Nucleotides, likewise, are referred to by their commonly accepted single-letter code.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “effective amount” or “therapeutically effective amount” refers to a quantity of an agent sufficient to achieve a desired therapeutic effect. In the context of therapeutic applications, the amount of a therapeutic peptide administered to the subject can depend on the type and severity of the infection and on the characteristics of the individual, such as general health, age, sex, body weight and tolerance to drugs. It can also depend on the degree, severity and type of disease. The skilled artisan will be able to determine appropriate dosages depending on these and other factors.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, S′ cap formation, and/or 3′ end formation) within a cell; (3) translation of an RNA sequence into a polypeptide or protein within a cell: (4) post-translational modification of a polypeptide or protein within a cell; (5) presentation of a polypeptide or protein on the cell surface; and (6) secretion or presentation or release of a polypeptide or protein from a cell.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 of amino acid sequences.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)2, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), V_{NAR} fragments, Bi-specific T-cell engager antibodies, minibodies, disulfide-linked Fvs (sdFv), and anti-idiotypic (anti-Id) antibodies, intrabodies, fusion polypeptides, unconventional antibodies and antigen-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4. IgA1 and IgA2) or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as Va) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3. FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system. As used herein interchangeably, the terms “antigen-binding portion”, “antigen-binding fragment”, or “antigen-binding region” of an antibody, refer to the region or portion of an antibody that binds to the antigen and which confers antigen specificity to the antibody; fragments of antigen-binding proteins, for example, antibodies includes one or more fragments of an antibody that retain the ability to specifically bind to an antigen (e.g., an peptide/HLA complex). It has been shown that the antigen-binding function of an antibody can be performed by fragments of a full-length antibody. Examples of antigen-binding portions encompassed within the term “antibody fragments” of an antibody include a Fab fragment, a monovalent fragment consisting of the V_{L}, V_{H}, C_{L} and CHI domains; a F(ab)₂ fragment, a bivalent fragment comprising two Fab fragments linked by a disulfide bridge at the hinge region; a Fd fragment consisting of the V_{H} and CHI domains: a Fv fragment consisting of the V_{L} and V_{H} domains of a single arm of an antibody; a dAb fragment (Ward et al., Nature 341:544-546 (1989)), which consists of a Vu domain; and an isolated complementarity determining region (CDR).

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85:5879-5883 (1988). These antibody fragments are obtained using conventional techniques known to those of ordinary skill in the art, and the fragments are screened for utility in the same manner as are intact antibodies.

An “isolated antibody” or “isolated antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}:V_{L} heterodimer. The heavy (Var) and light chains (V_{L}) are either joined directly or joined by a peptide-encoding linker (e.g., about 10, 15, 20, 25 amino acids), which connects the N-terminus of the Va with the C-terminus of the V_{H}, or the C-terminus of the Vu with the N-terminus of the V_{L}. The linker is usually rich in glycine for flexibility, as well as serine or threonine for solubility. The linker can link the heavy chain variable region and the light chain variable region of the extracellular antigen-binding domain.

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al., Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012): Shich et al., J Imunol 183(4):2277-85 (2009); Giomarelli et al., Thromb Haemosi 97(6):955-63 (2007); Fife et al., J Clin Invst 116(8):2252-61 (2006); Brocks et al., Immunotechnology 3(3): 173-84 (1997); Moosmayer et al., Ther Immunol 2(10):31-40 (1995) Agonistic scFvs having stimulatory activity have been described (see, e.g., Peter et al., J Biol Chem 25278(38):36740-7 (2003); Xie et al., Nat Biotech 15 (8): 768-71 (1997); Ledbetter et al., Crit Rev Immunol 17 (5-6): 427-55 (1997); Ho et al., Bio Chim Biophys Acta 1638(3):257-66 (2003)).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein. “F(ab)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al., Sequences of Proteins of Immunological Interest, Fifth Edition, U.S. Department of Health and Human Services, NIH Publication No. 91-3242(1991)).

As used herein, the term “constant region” or “constant domain” is interchangeable and has its meaning common in the art. The constant region is an antibody portion, e.g., a carboxyl terminal portion of a light and/or heavy chain which is not directly involved in binding of an antibody to antigen but which can exhibit various effector functions, such as interaction with the Fe receptor. The constant region of an immunoglobulin molecule generally has a more conserved amino acid sequence relative to an immunoglobulin variable domain.

As used herein, an “epitope” is a term in the art and can refer to a localized region of an antigen to which an antibody can immunospecifically bind. An epitope can be, e.g., contiguous amino acids of a polypeptide (linear or contiguous epitope) or an epitope can, e.g., come together from two or more noncontiguous regions of a polypeptide or polypeptides (conformational, non-linear, discontinuous, or non-contiguous epitope).

As used herein, the term “ligand” refers to a molecule that binds to a receptor. In particular, the ligand binds a receptor on another cell, allowing for cell-to-cell recognition and/or interaction.

As used herein, the term “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity.” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay). Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen-binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); Kimmel, A. R., Methods Enzymol. 152:507 (1987)).

As used herein, the terms “immunospecifically binds.” “immunospecifically recognizes,” “specifically binds,” and “specifically recognizes” are analogous terms in the context of antibodies and refer to antibodies and antigen-binding fragments thereof that bind to an antigen (e.g., epitope or immune complex) via the antigen-binding sites as understood by one skilled in the art, and does not exclude cross-reactivity of the antibody or antigen-binding fragment with other antigens.

The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino acids, to replace homologous sequences.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

As used herein, the term “analog” refers to a structurally related polypeptide or nucleic acid molecule having the function of a reference polypeptide or nucleic acid molecule.

As used herein, the term “a conservative sequence modification” refers to an amino acid modification that does not significantly affect or alter the binding characteristics of the presently disclosed anti-MUC16 antibody agent or an antigen-binding fragment thereof comprising the amino acid sequence. Conservative modifications can include amino acid substitutions, additions and deletions. Modifications can be introduced into the human scFv of the presently disclosed anti-MUC16 antibody or an antigen-binding fragment thereof by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%), 1%), 0.5%), or 0.1%) of other material, e.g., cellular material, culture medium, other nucleic acid molecules, chemical precursors and/or other chemicals.

As used herein, the term “isolated cell” refers to a cell that is separated from the molecular and/or cellular components that naturally accompany the cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The effective amount is generally determined by the physician on a case-by-case basis and is within the skill of one in the art. Several factors are typically considered when determining an appropriate dosage to achieve an effective amount. These factors include age, sex and weight of the subject, the condition being treated, the severity of the condition and the form and effective concentration of the engineered immune cells administered.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

### Anti-MUC16 Antibody Agents

Provided herein are anti-MUC16 antibody agents that immunospecifically bind to MUC16. In some embodiments, the anti-MUC16 antibody agent immunospecifically binds to the retained extracellular domain of MUC16. In some embodiments, the anti-MUC16 antibody agent is an anti-MUC16 construct that comprises an antibody moiety that immunospecifically binds to MUC16. In some embodiments, the anti-MUC16 antibody agent is an anti-MUC16 antibody (e.g., a full-length anti-MUC16 antibody or an antigen binding fragment thereof). In some embodiments, the anti-MUC16 antibody agent binds to an MUC16-expressing cell (e.g., an MUC16-expressing cancer cell).

Anti-MUC16 antibody agents, such as anti-MUC16 antibodies or antigen-binding fragments thereof, can include, e.g., monoclonal antibodies, polyclonal antibodies, recombinantly produced antibodies, monospecific antibodies, multispecific antibodies (including bispecific antibodies (BsAb)), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, single domain antibodies, monovalent antibodies, single chain antibodies or single-chain variable fragments (scFv), camelized antibodies, affybodies, and disulfide-linked Fvs (dsFv), Fc fusion proteins, immunoconjugates, or fragments thereof. Such antibodies and antigen-binding fragments can be made by methods known in the art.

In some embodiments, the anti-MUC16 antibody agent is a full-length antibody (e.g., full-length IgG) or antigen-binding fragment thereof, which specifically binds to MUC16.

In some embodiments, reference to an antibody agent that immunospecifically binds to MUC16 means that the antibody agent binds to MUC16 with an affinity that is at least about 10 times (including for example at least about any of 10, 10², 10³, 10⁴, 10⁵, 10⁶, or 10⁷ times) its binding affinity for non-target. In some embodiments, the non-target is an antigen that is not MUC16. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example, Biacore instruments, or kinetic exclusion assay (KinExA) utilizing, for example, Sapidyne instruments.

Although anti-MUC16 antibody agents containing human sequences (e.g., human heavy and light chain variable domain sequences comprising human CDR sequences) are extensively discussed herein, non-human anti-MUC16 antibody agents are also contemplated. In some embodiments, non-human anti-MUC16 antibody agents comprise human CDR sequences from an anti-MUC16 antibody agent as described herein and non-human framework sequences. Non-human framework sequences include, in some embodiments, any sequence that can be used for generating synthetic heavy and/or light chain variable domains using one or more human CDR sequences as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. In some embodiments, a non-human anti-MUC16 antibody agent includes an anti-MUC16 antibody agent generated by grafting one or more human CDR sequences as described herein onto a non-human framework sequence (e.g., a mouse or chicken framework sequence).

The complete amino acid sequence of an exemplary human MUC16 comprises or consists of the amino acid sequence of SEQ ID NO: 1. In some embodiments, the anti-MUC16 antibody agent described herein specifically recognizes an epitope within human MUC16. In some embodiments, the anti-MUC16 antibody agent described herein specifically recognizes an epitope within the retained extracellular domain of human MUC16. In some embodiments, the anti-MUC16 antibody agent described herein immunospecifically binds to that MUC16 ectodomain (FIG. 1). In some embodiments, the anti-MUC16 antibody agent described herein immunospecifically binds to a cell expressing human MUC16. In some embodiments, the anti-MUC16 antibody agent described herein immunospecifically binds to a cell expressing a recombinant MUC16 polypeptide. In some embodiments, the MUC16 polypeptide is MUC16-c344 having the amino acid sequence set forth in SEQ ID NO:24. In some embodiments, the MUC16 polypeptide is MUC16-c114 having the amino acid sequence set forth in SEQ ID NO: 25.

In some embodiments, the anti-MUC16 antibody agent cross-reacts with MUC16 polypeptide from a species other than human. In some embodiments, the anti-MUC16 antibody agent is completely specific for human MUC16 and does not exhibit species or other types of non-human cross-reactivity.

In some embodiments, the anti-MUC16 antibody agent specifically recognizes MUC16 expressed on the cell surface of a cancer cell (such as solid tumor). In some embodiments, the anti-MUC16 antibody agent specifically recognizes MUC16 expressed on the cell surface of one or more of ovarian cancer cells, breast cancer cells, prostate cancer cells, colon cancer cells, lung cancer cells, brain cancer cells, pancreatic cancer cells, kidney cancer cells, fallopian tube cancer cells, uterine (e.g., endometrial) cancer cells, primary peritoneum cancer cells or cancer cells of any other tissue that expresses MUC16. In some embodiments, the anti-MUC16 antibody agent specifically recognizes MUC16 expressed on the cell surface of a cancer cell line, e.g. ovarian cancer cell lines, such as OVCAR3, OVCA-432, OVCA-433 and CAOV3.

In some embodiments, the anti-MUC16 antibody agent cross-reacts with at least one allelic variant of the MUC16 protein, or fragments thereof. In some embodiments, the allelic variant has up to about 30, such as about any of 1, 2, 3, 4, 5, 6, 7, 8, 9, 10, 15, 20, 25, or 30, amino acid substitutions, such as a conservative amino acid substitution, when compared to the naturally occurring MUC16, or fragments thereof. In some embodiments, the anti-MUC16 antibody agent does not cross-react with any allelic variant of the MUC16 protein, or fragments thereof.

In some embodiments, the anti-MUC16 antibody agent cross-reacts with at least one interspecies variant of the MUC16 protein. In some embodiments, for example, the MUC16 protein, or fragments thereof, is human MUC16 and the interspecies variant of the MUC16 protein, or fragments thereof, is a mouse or rat variant thereof. In some embodiments, the anti-MUC16 antibody agent does not cross-react with any interspecies variant of the MUC16 protein.

In some embodiments, according to any of the anti-MUC16 antibody agents described herein, the anti-MUC16 antibody agent comprises an anti-MUC16 antibody moiety that specifically binds to MUC16. In some embodiments, the anti-MUC16 antibody moiety comprises an antibody heavy chain constant region and an antibody light chain constant region.

In some embodiments, the anti-MUC16 antibody moiety comprises an IgG1 heavy chain constant region. In some embodiments, the anti-MUC16 antibody moiety comprises an IgG2 heavy chain constant region. In some embodiments, the anti-MUC16 antibody moiety comprises an IgG3 heavy chain constant region.

In some embodiments, the anti-MUC16 antibody moiety comprises an IgG1 heavy chain constant region. In some embodiments, the heavy chain constant region comprises or consists of the amino acid sequence of SEQ ID NO: 28.

In some embodiments, the anti-MUC16 antibody moiety comprises an IgG4 heavy chain constant region. In some embodiments, the IgG4 heavy chain constant region comprises or consists of the amino acid sequence of SEQ ID NO: 29.

In some embodiments, the anti-MUC16 antibody moiety comprises a lambda light chain constant region. In some embodiments, the light chain constant region comprises or consists of the amino acid sequence of SEQ ID NO: 30.

In some embodiments, the anti-MUC16 antibody moiety comprises a kappa light chain constant region.

In some embodiments, the anti-MUC16 antibody moiety comprises an antibody heavy chain variable domain and an antibody light chain variable domain.

In some embodiments, the anti-MUC16 antibody moiety comprises a heavy chain variable domain comprising one, two or three HC-CDRs of SEQ ID NO: 2. In some embodiments, the anti-MUC16 antibody moiety comprises a heavy chain variable domain comprising HC-CDR1, HC-CDR2 and HC-CDR3 of the heavy chain variable domain of SEQ ID NO: 2. In some embodiments, the anti-MUC16 antibody moiety comprises a heavy chain variable domain comprising a HC-CDR1, HC-CDR2 and HC-CDR3 set forth in SEQ ID NOS: 4, 5, and 6, respectively. In some embodiments, the anti-MUC16 antibody moiety comprises a heavy chain variable domain comprising SEQ ID NO: 2. In some embodiments, the anti-MUC16 antibody moiety comprises a heavy chain variable domain set forth in SEQ ID NO: 2.

In some embodiments, the anti-MUC16 antibody moiety comprises a light chain variable domain comprising one, two or three LC-CDRs of SEQ ID NO: 3. In some embodiments, the anti-MUC16 antibody moiety comprises a light chain variable domain comprising LC-CDR1, LC-CDR2 and LC-CDR3 of the light chain variable domain of SEQ ID NO: 3. In some embodiments, the anti-MUC16 antibody moiety comprises a light chain variable domain comprising a LC-CDR1, LC-CDR2 and LC-CDR3 set forth in SEQ ID NOS: 7, 8, and 9, respectively. In some embodiments, the anti-MUC16 antibody moiety comprises a light chain variable domain comprising SEQ ID NO: 3. In some embodiments, the anti-MUC16 antibody moiety comprises a light chain variable domain set forth in SEQ ID NO: 3.

In some embodiments, the anti-MUC16 antibody moiety comprises a heavy chain variable domain comprising HC-CDR1, HC-CDR2 and HC-CDR3 of the heavy chain variable domain of SEQ ID NO: 2, and a light chain variable domain comprising LC-CDR1, LC-CDR2 and LC-CDR3 of the light chain variable domain of SEQ ID NO: 3. In some embodiments, the anti-MUC16 antibody moiety comprises a heavy chain variable domain comprising set forth in SEQ ID NOS: 4.5, and 6, respectively, and a light chain variable domain comprising LC-CDR1, LC-CDR2 and LC-CDR3 set forth in SEQ ID NOS: 7, 8, and 9, respectively. In some embodiments, the anti-MUC16 antibody moiety comprises a heavy chain variable domain comprising SEQ ID NO: 2, and a light chain variable domain comprising SEQ ID NO: 3. In some embodiments, the anti-MUC16 antibody moiety comprises a heavy chain variable domain set forth in SEQ ID NO: 2, and a light chain variable domain set forth in SEQ ID NO: 3.

In some embodiments, the antibody heavy chain variable domain comprises the amino acid sequence of SEQ ID NO: 2, or a variant thereof comprising up to about 5 (such as about any of 1, 2, 3, 4, or 5) amino acid substitutions or having at least about 95% (for example at least about any of 96%, 97%, 98%, or 99%) sequence identity to SEQ ID NO: 2. In some embodiments, the light chain variable domain comprises the amino acid sequence of SEQ ID NO: 3, or a variant thereof comprising up to about 5 (such as about any of 1, 2, 3, 4, or 5) amino acid substitutions or having at least about 95% (for example at least about any of 96%, 97%, 98%, or 99%) sequence identity to SEQ ID NO: 3.

In some embodiments, the anti-MUC16 antibody moiety comprises a heavy chain variable domain comprising one, two or three HC-CDRs of SEQ ID NO: 10. In some embodiments, the anti-MUC16 antibody moiety comprises a heavy chain variable domain comprising HC-CDR1, HC-CDR2 and HC-CDR3 of the heavy chain variable domain of SEQ ID NO: 10. In some embodiments, the anti-MUC16 antibody moiety comprises a heavy chain variable domain comprising a HC-CDR1, HC-CDR2 and HC-CDR3 set forth in SEQ ID NOS: 12, 13, and 14, respectively. In some embodiments, the anti-MUC16 antibody moiety comprises a heavy chain variable domain comprising SEQ ID NO: 10. In some embodiments, the anti-MUC16 antibody moiety comprises a heavy chain variable domain set forth in SEQ ID NO: 10.

In some embodiments, the anti-MUC16 antibody moiety comprises a light chain variable domain comprising one, two or three LC-CDRs of SEQ ID NO: 11. In some embodiments, the anti-MUC16 antibody moiety comprises a light chain variable domain comprising LC-CDR1, LC-CDR2 and LC-CDR3 of the light chain variable domain of SEQ ID NO: 11. In some embodiments, the anti-MUC16 antibody moiety comprises a light chain variable domain comprising a LC-CDR1, LC-CDR2 and LC-CDR3 set forth in SEQ ID NOS: 15, 16, and 17, respectively. In some embodiments, the anti-MUC16 antibody moiety comprises a light chain variable domain comprising SEQ ID NO: 11. In some embodiments, the anti-MUC16 antibody moiety comprises a light chain variable domain set forth in SEQ ID NO: 1.

In some embodiments, the anti-MUC16 antibody moiety comprises a heavy chain variable domain comprising HC-CDR1, HC-CDR2 and HC-CDR3 of the heavy chain variable domain of SEQ ID NO: 10, and a light chain variable domain comprising LC-CDR1, LC-CDR2 and LC-CDR3 of the light chain variable domain of SEQ ID NO: 11. In some embodiments, the anti-MUC16 antibody moiety comprises a heavy chain variable domain comprising set forth in SEQ ID NOS: 12, 13, and 14, respectively, and a light chain variable domain comprising LC-CDR1, LC-CDR2 and LC-CDR3 set forth in SEQ ID NOS: 15, 16, and 17, respectively. In some embodiments, the anti-MUC16 antibody moiety comprises a heavy chain variable domain comprising SEQ ID NO: 10, and a light chain variable domain comprising SEQ ID NO: 11. In some embodiments, the anti-MUC16 antibody moiety comprises a heavy chain variable domain set forth in SEQ ID NO: 10, and a light chain variable domain set forth in SEQ ID NO: 11.

In some embodiments, the antibody heavy chain variable domain comprises the amino acid sequence of SEQ ID NO: 10, or a variant thereof comprising up to about 5 (such as about any of 1, 2, 3, 4, or 5) amino acid substitutions or having at least about 95% (for example at least about any of 96%, 97%, 98%, or 99%) sequence identity to SEQ ID NO: 10. In some embodiments, the light chain variable domain comprises the amino acid sequence of SEQ ID NO: 11, or a variant thereof comprising up to about 5 (such as about any of 1, 2, 3, 4, or 5) amino acid substitutions or having at least about 95% (for example at least about any of 96%, 97%, 98%, or 99%) sequence identity to SEQ ID NO: 11.

10113| Exemplary antibody sequences are shown in the Tables below. The exemplary CDR sequences in Table 2 are predicted using the IgBLAST algorithm. See, for example, Ye J. et al., Nucleic Acids Research 41:W34-W40 (2013), the disclosure of which is incorporated herein by reference in its entirety. Those skilled in the art will recognize that many algorithms are known for prediction of CDR positions in antibody heavy chain and light chain variable regions, and antibody agents comprising CDRs from antibodies described herein, but based on prediction algorithms other than IgBLAST, are within the scope of this invention.

The exemplary antibody heavy chain and light chain variable region sequences are delimited according to the INTERNATIONAL IMMUNOGENETICS INFORMATION SYSTEM® (IMGT). See, for example, Lefranc, M.-P. et al., Nucleic Acids Res., 43: D413-422 (2015), the disclosure of which is incorporated herein by reference in its entirety. Those skilled in the art will recognize that antibody agents comprising V_{H} or V_{L} sequences from antibodies described herein, but based on algorithms other than IMGT, are within the scope of this invention.

### Table 2. Exemplary Anti-MUC16 Antibody CDR Sequences.

TABLE 2
Exemplary anti-MUC16 antibody CDR sequences.

| Clone | | | |
|---|---|---|---|
| ID | HC-CDR1 | HC-CDR2 | HC-CDR3 |
| 8 | GGSFSGYY | INHSGST | ARQSYITDS |
| | (SEQ ID | (SEQ ID NO: 5) | (SEQ ID NO: 6) |
| | NO: 4) | | |
| 12 | GGSFSGYY | INHSGST | ARWSPFSYKQMYDY |
| | (SEQ ID | (SEQ ID NO: 13) | (SEQ ID NO: 14) |
| | NO: 12) | | |
| Clone | | | |
| ID | LC-CDR1 | LC-CDR2 | LC-CDR3 |
| 8 | QDVSKW | AAS | QQANSFPWT |
| | (SEQ ID | (SEQ ID NO: 8) | (SEQ ID NO: 9) |
| | NO: 7) | | |
| 12 | RGSIASAY | EDY | QSYDDNDHVI |
| | (SEQ ID | (SEQ ID NO: 16) | (SEQ ID NO: 17) |
| | NO: 15) | | |

### Table 3. Exemplary Anti-MUC16 Antibody VH and VL Domain Sequences.

TABLE 3
Exemplary anti-MUC16 antibody
VH and VL domain sequences.

| Clone | | |
|---|---|---|
| ID | Description | Sequence |
| 8 | VH domain | QVQLQQWGAGLLKPSETLSLTCAVYGGSESG |
| | | YYWSWIRQPPGKGLEWIGEINHSGSTNYNPS |
| | | LKSRVTISVDTSKNQFSLKLSSVTAADTAVY |
| | | YCARQSYITDSWGQGTLVTVSS |
| | | (SEQ ID NO: 2) |
| 8 | VL | DIQLTQSPSAVSASVGDRVTITCRASQDVSK |
| | domain | WLAWYQQKPGKAPRLLISAASGLQSWVPSRF |
| | | SGSGSGTEFTLSISSLQPEDFATYYCQQANS |
| | | FPWTFGQGTKVEIKR |
| | | (SEQ ID NO: 3) |
| 12 | VH domain | QVQLQQWGAGLLKPSETLSLICAVYGGSFSG |
| | | YYWSWIRQPPGKGLEWIGEINHSGSTNYNPS |
| | | LKSRIIMSVDTSKRQFSLKLRSATAADTAVY |
| | | YCARWSPFSYKQMYDYWGQGTLVTVSS |
| | | (SEQ ID NO: 10) |
| 12 | VL | NFMLTQPHSVSESPGKTVTISCTRSRQSIAS |
| | domain | AYVQWYQQRPGSAPITVIYEDYERPSEIPDR |
| | | FSGSIDSSSNSASLTISGLKTEDEADYYCQS |
| | | YDDNDHVIFGGGTKVTVLG |
| | | (SEQ ID NO: 11) |

### Full-Length Anti-MUC16 Antibody

The anti-MUC16 antibody agent in some embodiments is a full-length anti-MUC16 antibody. In some embodiments, the full-length anti-MUC16 antibody is an IgA, IgD, IgE, IgG, or IgM. In some embodiments, the full-length anti-MUC16 antibody comprises IgG constant domains, such as constant domains of any of IgG1, IgG2, IgG3, and IgG4 including variants thereof. In some embodiments, the full-length anti-MUC16 antibody comprises a lambda light chain constant region. In some embodiments, the full-length anti-MUC16 antibody comprises a kappa light chain constant region. In some embodiments, the full-length anti-MUC16 antibody is a full-length human anti-MUC16 antibody. In some embodiments, the full-length anti-MUC16 antibody comprises an Fc sequence of a mouse immunoglobulin. In some embodiments, the full-length anti-MUC16 antibody comprises an Fc sequence that has been altered or otherwise changed so that it has enhanced antibody dependent cellular cytotoxicity (ADCC) or complement dependent cytotoxicity (CDC) effector function.

Thus, for example, in some embodiments, there is provided a full-length anti-MUC16 antibody comprising IgG1 or IgG4 constant domains, wherein the anti-MUC16 antibody specifically binds to MUC16 on a tumor cell. In some embodiments, the IgG1 is human IgG1. In some embodiments, the IgG1 is human IgG4. In some embodiments, the anti-MUC16 heavy chain constant region comprises or consists of the amino acid sequence of SEQ ID NO: 28 or 29. In some embodiments, the anti-MUC16 light chain constant region comprises or consists of the amino acid sequence of SEQ ID NO: 30. In some embodiments, the anti-MUC16 heavy chain constant region comprises or consists of the amino acid sequence of SEQ ID NO: 28 or 29 and the anti-MUC16 light chain constant region comprises or consists of the amino acid sequence of SEQ ID NO: 30. In some embodiments, binding of the anti-MUC16 antibody to an MUC10-expressing cell (e.g., an MUC16-expressing cancer cell) inhibits tumor growth or metastasis of a tumor or induces regression of a tumor. In some embodiments, binding of the anti-MUC16 antibody to an MUC16-expressing cell (e.g., an MUC16-expressing cancer cell) inhibits Matrigel invasion in vitro of the MUC16-expressing cells.

In some embodiments, there is provided a full-length anti-MUC16 antibody comprising IgG1 or IgG4 constant domains, wherein the anti-MUC16 antibody comprises a) a heavy chain variable domain comprising an HC-CDR1 comprising the amino acid sequence of SEQ ID NO: 4, an HC-CDR2 comprising the amino acid sequence of SEQ ID NO: 5, and an HC-CDR3 comprising the amino acid sequence of SEQ ID NO: 6; and b) a light chain variable domain comprising an LC-CDR1 comprising the amino acid sequence of SEQ ID NO: 7, an LC-CDR2 comprising the amino acid sequence of SEQ ID NO: 8, and an LC-CDR3 comprising the amino acid sequence of SEQ ID NO: 9. In some embodiments, the IgG1 is human IgG1. In some embodiments, the IgG4 is human IgG4. In some embodiments, the anti-MUC16 heavy chain constant region comprises or consists of the amino acid sequence of SEQ ID NO: 28 or 29. In some embodiments, the anti-MUC16 light chain constant region comprises or consists of the amino acid sequence of SEQ ID NO: 30.

In some embodiments, there is provided a full-length anti-MUC16 antibody comprising IgG1 or IgG4 constant domains, wherein the anti-MUC16 antibody comprises a) a heavy chain variable domain comprising an HC-CDR1 comprising the amino acid sequence of SEQ ID NO: 12, an HC-CDR2 comprising the amino acid sequence of SEQ ID NO: 13, and an HC-CDR3 comprising the amino acid sequence of SEQ ID NO: 14; and b) a light chain variable domain comprising an LC-CDR1 comprising the amino acid sequence of SEQ ID NO: 15, an LC-CDR2 comprising the amino acid sequence of SEQ ID NO: 16, and an LC-CDR3 comprising the amino acid sequence of SEQ ID NO: 17. In some embodiments, the IgG1 is human IgG1. In some embodiments, the IgG4 is human IgG4. In some embodiments, the anti-MUC16 heavy chain constant region comprises or consists of the amino acid sequence of SEQ ID NO: 28 or 29. In some embodiments, the anti-MUC16 light chain constant region comprises or consists of the amino acid sequence of SEQ ID NO: 30.

### Chimeric Anti-MUC16 Constructs

In some embodiments, the anti-MUC16 antibody agent is an anti-MUC16 chimeric antigen receptor (CAR) or variant thereof that specifically binds to MUC16. In some embodiments, the anti-MUC16 antibody agent is an anti-MUC16 CAR. CARs are well known in the art, and the anti-MUC16 antibody agent can be a CAR according to any CAR known in the art, such as described in Sadelain et al., Nature 545:423-431 (2017), the disclosure of which is explicitly incorporated herein for use in the present invention and for possible inclusion in one or more claims herein. In some embodiments, the anti-MUC16 CAR comprises an anti-MUC16 antibody moiety according to any of the anti-MUC16 antibody moieties described herein. For example, in some embodiments, there is provided an anti-MUC16 CAR comprising an anti-MUC16 antibody moiety. In some embodiments, the anti-MUC16 antibody moiety of an anti-MUC16 CAR comprises a) an antibody heavy chain variable domain comprising an HC-CDR1 comprising the amino acid sequence of SEQ ID NO: 4, an HC-CDR2 comprising the amino acid sequence of SEQ ID NO: 5, and an HC-CDR3 comprising the amino acid sequence of SEQ ID NO: 6; and b) a light chain variable domain comprising an LC-CDR1 comprising the amino acid sequence of SEQ ID NO: 7, an LC-CDR2 comprising the amino acid sequence of SEQ ID NO: 8, and an LC-CDR3 comprising the amino acid sequence of SEQ ID NO: 9. In some embodiments, the heavy chain variable domain comprises the amino acid sequence of SEQ ID NO: 2, or a variant thereof having at least about 95% (for example at least about any of 96%, 97%, 98%, or 99%) sequence identity, and the light chain variable domain comprises the amino acid sequence of SEQ ID NO: 3, or a variant thereof having at least about 95% sequence identity.

In some embodiments, the anti-MUC16 antibody moiety of an anti-MUC16 CAR comprises a) a heavy chain variable domain comprising an HC-CDR1 comprising the amino acid sequence of SEQ ID NO: 12, an HC-CDR2 comprising the amino acid sequence of SEQ ID NO: 13, and an HC-CDR3 comprising the amino acid sequence of SEQ ID NO: 14; and b) a light chain variable domain comprising an LC-CDR1 comprising the amino acid sequence of SEQ ID NO: 15, an LC-CDR2 comprising the amino acid sequence of SEQ ID NO: 16, and an LC-CDR3 comprising the amino acid sequence of SEQ ID NO: 17. In some embodiments, the heavy chain variable domain comprises the amino acid sequence of SEQ ID NO: 10, or a variant thereof having at least about 95% (for example at least about any of 96%, 97%, 98%, or 99%) sequence identity, and the light chain variable domain comprises the amino acid sequence of SEQ ID NO: 11, or a variant thereof having at least about 95% sequence identity.

In some embodiments, the anti-MUC16 antibody agent is an anti-MUC16 chimeric receptor comprising T cell receptor (TCR) transmembrane domains. For example, in some embodiments, the anti-MUC16 antibody agent is an antibody-T cell receptor (abTCR) as described in PCT Patent Application Publication No. WO2017070608, the disclosure of which is explicitly incorporated herein for use in the present invention and for possible inclusion in one or more claims herein. In some embodiments, the anti-MUC16 abTCR comprises an anti-MUC16 antibody moiety according to any of the anti-MUC16 antibody moieties described herein. For example, in some embodiments, there is provided an anti-MUC16 abTCR comprising an anti-MUC16 antibody moiety.

In some embodiments, the anti-MUC16 antibody moiety of an anti-MUC16 abTCR comprises a) an antibody heavy chain variable domain comprising an HC-CDR1 comprising the amino acid sequence of SEQ ID NO: 4, an HC-CDR2 comprising the amino acid sequence of SEQ ID NO: 5, and an HC-CDR3 comprising the amino acid sequence of SEQ ID NO: 6; and b) a light chain variable domain comprising an LC-CDR1 comprising the amino acid sequence of SEQ ID NO: 7, an LC-CDR2 comprising the amino acid sequence of SEQ ID NO: 8, and an LC-CDR3 comprising the amino acid sequence of SEQ ID NO: 9. In some embodiments, the heavy chain variable domain of an anti-MUC16 abTCR comprises the amino acid sequence of SEQ ID NO: 2, or a variant thereof having at least about 95% (for example at least about any of 96%, 97%, 98%, or 99%) sequence identity, and the light chain variable domain comprises the amino acid sequence of SEQ ID NO: 3, or a variant thereof having at least about 95% sequence identity.

In some embodiments, the anti-MUC16 antibody moiety of an anti-MUC16 abTCR comprises a) a heavy chain variable domain comprising an HC-CDR1 comprising the amino acid sequence of SEQ ID NO: 12, an HC-CDR2 comprising the amino acid sequence of SEQ ID NO: 13, and an HC-CDR3 comprising the amino acid sequence of SEQ ID NO: 14; and b) a light chain variable domain comprising an LC-CDR1 comprising the amino acid sequence of SEQ ID NO: 15, an LC-CDR2 comprising the amino acid sequence of SEQ ID NO: 16, and an LC-CDR3 comprising the amino acid sequence of SEQ ID NO: 17. In some embodiments, the heavy chain variable domain of an anti-MUC16 abTCR comprises the amino acid sequence of SEQ ID NO: 10, or a variant thereof having at least about 95% (for example at least about any of 96%, 97%, 98%, or 99%) sequence identity, and the light chain variable domain comprises the amino acid sequence of SEQ ID NO: 11, or a variant thereof having at least about 95% sequence identity.

In some embodiments, the anti-MUC16 antibody agent is a chimeric co-stimulatory receptor comprising an anti-MUC16 antibody moiety that specifically binds to MUC16 and a co-stimulatory signaling domain. In some embodiments, the anti-MUC16 chimeric co-stimulatory receptor is capable of stimulating an immune cell on the surface of which it is functionally expressed upon binding MUC16. In some embodiments, the anti-MUC16 chimeric co-stimulatory receptor lacks a functional primary immune cell signaling sequence. In some embodiments, the anti-MUC16 chimeric co-stimulatory receptor lacks any primary immune cell signaling sequence. In some embodiments, the anti-MUC16 chimeric co-stimulatory receptor comprises a single polypeptide chain comprising the anti-MUC16 antibody moiety, a transmembrane domain, and the co-stimulatory signaling domain. In some embodiments, the anti-MUC16 chimeric co-stimulatory receptor comprises a first polypeptide chain and a second polypeptide chain, wherein the first and second polypeptide chains together form the anti-MUC16 antibody moiety, a transmembrane module, and co-stimulatory signaling module comprising the co-stimulatory signaling domain. In some embodiments, the first and second polypeptide chains are separate polypeptide chains, and the anti-MUC16 chimeric co-stimulatory receptor is a multimer, such as a dimer. In some embodiments, the first and second polypeptide chains are covalently linked, such as by a peptide linkage, or by another chemical linkage, such as a disulfide linkage. In some embodiments, the first polypeptide chain and the second polypeptide chain are linked by at least one disulfide bond. In some embodiments, the anti-MUC16 antibody moiety is a Fab, a Fab′, a (Fab′) 2, an Fv, or a single chain Fv (scFv).

Examples of co-stimulatory immune cell signaling domains for use in the anti-MUC16 chimeric co-stimulatory receptors of the invention include the cytoplasmic sequences of co-receptors of the T cell receptor (TCR), which can act in concert with a chimeric receptor (e.g., a CAR or abTCR) to initiate signal transduction following chimeric receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of intracellular signaling sequence: those that initiate antigen-dependent primary activation through the TCR (referred to herein as “primary immune cell signaling sequences”) and those that act in an antigen-independent manner to provide a secondary or co-stimulatory signal (referred to herein as “co-stimulatory immune cell signaling sequences”).

Primary immune cell signaling sequences that act in a stimulatory manner may contain signaling motifs which are known as immunoreceptor tyrosine-based activation motifs or ITAMs. Examples of ITAM-containing primary immune cell signaling sequences include those derived from TCRζ, FcRγ, FcRβ, CD3γ, CD3δ, CD3ε, CD5, CD22, CD79a, CD79b, and CD66d. A “functional” primary immune cell signaling sequence is a sequence that is capable of transducing an immune cell activation signal when operably coupled to an appropriate receptor. “Non-functional” primary immune cell signaling sequences, which may comprise fragments or variants of primary immune cell signaling sequences, are unable to transduce an immune cell activation signal. The anti-MUC16 chimeric co-stimulatory receptors described herein lack a functional primary immune cell signaling sequence, such as a functional signaling sequence comprising an ITAM. In some embodiments, the anti-MUC16 chimeric co-stimulatory receptors lack any primary immune cell signaling sequence.

The co-stimulatory immune cell signaling sequence can be a portion of the intracellular domain of a co-stimulatory molecule including, for example, CD27, CD28, 4-1BB (CD137), OX40, CD30, CD40, PD-1, ICOS, lymphocyte function-associated antigen-1 (LFA-1), CD2. CD7, LIGHT, NKG2C, B7-H3, a ligand that specifically binds with CD83, and the like.

In some embodiments, the anti-MUC16 antibody moiety of an anti-MUC16 chimeric co-stimulatory receptor comprises a) an HC-CDR1 comprising the amino acid sequence of SEQ ID NO: 4, an HC-CDR2 comprising the amino acid sequence of SEQ ID NO: S, and an HC-CDR3 comprising the amino acid sequence of SEQ ID NO: 6; and b) a light chain variable domain comprising an LC-CDR1 comprising the amino acid sequence of SEQ ID NO: 7, an LC-CDR2 comprising the amino acid sequence of SEQ ID NO: 8, and an LC-CDR3 comprising the amino acid sequence of SEQ ID NO: 9. In some embodiments, the heavy chain variable domain comprises the amino acid sequence of SEQ ID NO: 2, or a variant thereof having at least about 95% (for example at least about any of 96%, 97%, 98%, or 99%) sequence identity, and the light chain variable domain comprises the amino acid sequence of SEQ ID NO: 3, or a variant thereof having at least about 95% sequence identity.

In some embodiments, the anti-MUC16 antibody moiety of an anti-MUC16 chimeric co-stimulatory receptor comprises a) a heavy chain variable domain comprising an HC-CDR1 comprising the amino acid sequence of SEQ ID NO: 12, an HC-CDR2 comprising the amino acid sequence of SEQ ID NO: 13, and an HC-CDR3 comprising the amino acid sequence of SEQ ID NO: 14; and b) a light chain variable domain comprising an LC-CDR1 comprising the amino acid sequence of SEQ ID NO: 15, an LC-CDR2 comprising the amino acid sequence of SEQ ID NO: 16, and an LC-CDR3 comprising the amino acid sequence of SEQ ID NO: 17. In some embodiments, the heavy chain variable domain comprises the amino acid sequence of SEQ ID NO: 10, or a variant thereof having at least about 95% (for example at least about any of 96%, 97%, 98%, or 99%) sequence identity, and the light chain variable domain comprises the amino acid sequence of SEQ ID NO: 11, or a variant thereof having at least about 95% sequence identity.

In some embodiments, the anti-MUC16 chimeric co-stimulatory receptor is expressed in an immune cell. In some embodiments, the anti-MUC16 chimeric co-stimulatory receptor is expressed in an immune cell that expresses another chimeric receptor. In some embodiments, the other chimeric receptor is a CAR or an abTCR. In some embodiments, the other chimeric receptor binds to MUC16. In some embodiments, the other chimeric receptor does not bind to MUC16. In some embodiments, the other chimeric receptor binds to an antigen associated with a cancer characterized by high expression of MUC16 and/or high aerobic glycolysis. In some embodiments, the other chimeric receptor binds to an antigen associated with any of the cancers described herein (such as kidney cancer, cervical cancer, prostate cancer, breast cancer, colon cancer, brain cancer, or pancreatic cancer). In some embodiments, the other chimeric receptor binds to an antigen associated with kidney cancer. In some embodiments, the kidney cancer is renal cell carcinoma (RCC). In some embodiments, the RCC is metastatic RCC. In some embodiments, the immune cell is a T cell. In some embodiments, expression of the anti-MUC16 chimeric co-stimulatory receptor in the immune cell is inducible. In some embodiments, the expression of the anti-MUC16 chimeric co-stimulatory receptor in the immune cell is inducible upon signaling through the other chimeric receptor.

### Binding Affinity

Binding affinity can be indicated by K_{d}, K_{off}, Kₒₙ, or Kₐ. The term “K_{off}”, as used herein, is intended to refer to the off-rate constant for dissociation of an antibody agent from the antibody agent/antigen complex, as determined from a kinetic selection set up. The term “Kₒₙ”, as used herein, is intended to refer to the on-rate constant for association of an antibody agent to the antigen to form the antibody agent/antigen complex. The term equilibrium dissociation constant “K_{d}”, as used herein, refers to the dissociation constant of a particular antibody agent-antigen interaction, and describes the concentration of antigen required to occupy one half of all of the antibody-binding domains present in a solution of antibody agent molecules at equilibrium, and is equal to K_{off}/Kₒₙ The measurement of K_{d} presupposes that all binding agents are in solution. In the case where the antibody agent is tethered to a cell wall, e.g., in a yeast expression system, the corresponding equilibrium rate constant is expressed as EC50, which gives a good approximation of K_{d}. The affinity constant, K_{d}, is the inverse of the dissociation constant, K_{d}.

The dissociation constant (K_{d}) is used as an indicator showing affinity of antibody moieties to antigens. For example, easy analysis is possible by the Scatchard method using antibody agents marked with a variety of marker agents, as well as by using Biacore (made by Amersham Biosciences), analysis of biomolecular interactions by surface plasmon resonance, according to the user's manual and attached kit. The K_{d} value that can be derived using these methods is expressed in units of M (Mols). An antibody agent that specifically binds to a target may have a K_{d} of, for example, ≤10⁻⁷ M, ≤10⁻⁸ M, ≤10⁻⁹ M, ≤10⁻¹⁰ M, ≤10⁻¹¹ M, ≤10⁻¹² M, or ≤10⁻¹³ M.

Binding specificity of the antibody agent can be determined experimentally by methods known in the art. Such methods comprise, but are not limited to, Western blots, ELISA-, RIA-, ECL-, IRMA-, EIA-, BIAcore-tests and peptide scans. In some embodiments, the binding affinity of the anti-MUC16 antibody agent is measured by testing the binding affinity of the anti-MUC16 antibody agent to cells expressing MUC16 on the surface (e.g., HepG2 cells).

In some embodiments, the anti-MUC16 antibody agent specifically binds to a target MUC16 (e.g., nMUC16) with a K_{d} of about 10⁻⁷ M to about 10⁻¹³ M (such as about 10⁻⁷ M to about 10⁻¹³ M, about 10⁻⁹ M to about 10⁻¹³ M, or about 10⁻¹⁰ M to about 10⁻¹² M). Thus in some embodiments, the K_{d} of the binding between the anti-nMUC16 antibody agent and nMUC16, the K_{d} of the binding between the anti-sMUC16 antibody agent and sMUC16, or the K_{d} of the binding between the anti-MUC16 antibody agent and MUC16 (any format), is about 10⁻⁷ M to about 10⁻¹³ M, about 1×10⁻⁷ M to about 5×10⁻¹³ M, about 10⁻⁷ M to about 10⁻¹² M, about 10⁻⁷ M to about 10⁻¹¹ M, about 10⁻⁷ M to about 10⁻¹⁰ M, about 10⁻⁷ M to about 10⁻⁹ M, about 10⁻⁸ M to about 10⁻¹³ M, about 1×10⁻⁸ M to about 5×10⁻¹³ M, about 10⁻⁸ M to about 10⁻¹² M, about 10⁻⁸ M to about 10⁻¹¹ M, about 10⁻⁸ M to about 10⁻¹⁰ M, about 10⁻⁸ M to about 10⁻⁹ M, about 5×10⁻⁹ M to about 1×10⁻¹³ M, about 5×10⁻⁹ M to about 1×10⁻¹² M, about 5×10⁻⁹ M to about 1×10⁻¹¹ M, about 5×10⁻⁹ M to about 1×10⁻¹⁰ M, about 10⁻⁹ M to about 10⁻¹³ M, about 10⁻⁹ M to about 10⁻¹² M, about 10⁻⁹ M to about 10⁻¹¹ M, about 10⁻⁹ M to about 10⁻¹⁰ M, about 5×10⁻¹⁰ M to about 1×10⁻¹³ M, about 5×10⁻¹⁰ M to about 1×10⁻¹² M, about 5×10⁻¹⁰ M to about 1×10⁻¹¹ M, about 10⁻¹⁰ M to about 10⁻¹³ M, about 1×10⁻¹⁰ M to about 5×10⁻¹³ M, about 1×10⁻¹⁰ M to about 1×10⁻¹² M, about 1×10⁻¹⁰ M to about 5×10⁻¹² M, about 1×10⁻¹⁰ M to about 1×10⁻¹¹ M, about 10⁻¹¹ M to about 10⁻¹³ M, about 1×10⁻¹¹ M to about 5×10⁻¹³ M, about 10⁻¹¹ M to about 10⁻¹² M. or about 10⁻¹² M to about 10⁻¹³ M. In some embodiments, the K_{d} of the binding between the anti-uMUC16 antibody agent and an nMUC16 is about 10⁻⁷ M to about 10⁻¹³ M.

In some embodiments, the K_{d} of the binding between the anti-MUC16 antibody agent and a non-target is more than the K_{d} of the binding between the anti-MUC16 antibody agent and the target, and is herein referred to in some embodiments as the binding affinity of the anti-MUC16 antibody agent to the target (e.g., cell surface-bound MUC16) is higher than that to a non-target. In some embodiments, the non-target is an antigen that is not MUC16. In some embodiments, the K_{d} of the binding between the anti-MUC16 antibody agent (against nMUC16) and a non-MUC16 target can be at least about 10 times, such as about 10-100 times, about 100-1000 times, about 10³-10⁴ times, about 10⁴-10⁵ times, about 10⁵-10⁶ times, about 10⁶-10⁷ times, about 10⁷-10⁸ times, about 10⁸-10⁹ times, about 10⁹-10¹⁰ times, about 10¹⁰-10¹¹ times, or about 10¹¹-10¹² times of the K_{d} of the binding between the anti-MUC16 antibody agent and a target MUC16.

In some embodiments, the anti-MUC16 antibody agent binds to a non-target with a K_{d} of about 10⁻¹ M to about 10⁻⁶ M (such as about 10⁻⁴ M to about 10⁻⁶ M, about 10⁻⁴ M to about 10⁻⁵ M, or about 10⁻² M to about 10⁻⁴ M). In some embodiments, the non-target is an antigen that is not MUC16. Thus in some embodiments, the K_{d} of the binding between the anti-MUC16 antibody agent and a non-MUC16 target is about 10⁻¹ M to about 10⁻⁴ M, about 1×10⁻¹ M to about 5×10⁻⁶ M, about 10⁻¹ M to about 10⁻⁵ M, about 1×10⁻¹ M to about 5×10⁻⁵ M, about 10⁻¹ M to about 10⁻⁴ M, about 1×10⁻¹ M to about 5×10⁻¹ M, about 10⁻¹ M to about 10−3 M, about 1×10−M to about 5×10−3 M, about 10⁻¹ M to about 10⁻² M, about 10⁻² M to about 10⁻⁶ M, about 1×10⁻²M to about 5×10⁻⁶ M, about 10⁻² M to about 10⁻⁵ M, about 1×10⁻² M to about 5×10⁻⁵ M, about 10⁻² M to about 10⁻⁴ M, about 1×10⁻² M to about 5×10⁻⁴ M, about 10⁻² M to about 10−3 M, about 10⁻³ M to about 10⁻⁶ M, about 1×10⁻³ M to about 5×10⁻⁶ M, about 10⁻³ M to about 10⁻⁵ M, about 1×10⁻³ M to about 5×10⁻⁵ M, about 10⁻³ M to about 10⁻⁴ M, about 10⁻⁴ M to about 10⁻⁴ M, about 1×10⁻⁴ M to about 5×10⁻⁶ M, about 10⁻⁴ M to about 10⁻⁵ M, or about 10⁻⁵ M to about 10⁻⁶ M.

In some embodiments, when referring to that the anti-MUC16 antibody agent specifically recognizes a target MUC16 (e.g., cell surface-bound MUC16) at a high binding affinity, and binds to a non-target at a low binding affinity, the anti-MUC16 antibody agent will bind to the target MUC16 (e.g., cell surface-bound MUC16) with a K_{d} of about 10⁻⁷ M to about 10⁻¹³ M (such as about 10⁻⁷ M to about 10⁻¹³ M, about 10⁻⁹ M to about 10⁻¹³ M, or about 10⁻¹⁰ M to about 10⁻¹² M), and will bind to the non-target with a K_{d} of about 10⁻¹ M to about 10⁻⁶ M (such as about 10⁻¹ M to about 10⁻⁶ M, about 10⁻¹ M to about 10⁻⁵ M, or about 10⁻² M to about 10⁻⁴ M).

In some embodiments, when referring to that the anti-MUC16 antibody agent specifically recognizes a cell surface-bound MUC16, the binding affinity of the anti-MUC16 antibody agent is compared to a control anti-MUC16 antibody agent. In some embodiments, the K_{d} of the binding between the control anti-MUC16 antibody agent and a cell surface-bound MUC16 can be at least about 2 times, such as about 2 times, about 3 times, about 4 times, about 5 times, about 6 times, about 7 times, about & times, about 9 times, about 10 times, about 10-100 times, about 100-1000 times, about 10³-10⁴ times, about 10⁴-10⁵ times, about 10⁵-10⁶ times, about 10⁶-10⁷ times, about 10⁷-10⁸ times, about 10⁸-10⁹ times, about 10⁹-10¹⁰ times, about 10¹⁰-10¹¹ times, or about 10¹¹-10¹² times of the K_{d} of the binding between the anti-nMUC16 antibody agent described herein and a cell surface-bound MUC16.

### Functional Activities of Anti-Mue16 Antibody Agents

In certain embodiments, an anti-MUC16 antibody agent or an antigen-binding fragment thereof described herein inhibits Matrigel invasion in vitro of cells recombinantly expressing a MUC16 polypeptide. In some embodiments the MUC16 comprises SEQ ID NO: 25 (MUC16 c114). In certain embodiments, the cells recombinantly expressing glycosylated MUC16 c114 are SKOV3 cells. In certain embodiments, the MUC16 polypeptide is glycosylated. In certain embodiments, the glycosylated form of MUC16 polypeptide is N-glycosylated at amino acid residue Asn30 (corresponding to Asn1806 of mature MUC16 (SEQ ID NO: 1)). In certain embodiments, MUC16 polypeptide is N-glycosylated at amino acid residues Asn24 and Asn30 (corresponding to Asn1800 and Asn1806, respectively, of mature MUC16 (SEQ ID NO: 1)). In certain embodiments, the MUC16 polypeptide is N-glycosylated at amino acid residues Asn1, Asn24, and Asn30 of SEQ ID NO: 25 (also referred to as Asnl1777. Asn1800, and Asn1806, respectively, in Yin and Lloyd (2001) J Bio! Chem 276:27371-27375). In certain embodiments, the glycosylation comprises N-linked chitobiose. In certain embodiments, the glycosylation consists of an N-linked chitobiose. In certain embodiments, Matrigel invasion is inhibited by at least 1.25, 1.5, 1.75, 2, 3, 4, 5, 6, 7, 8, 9, or 10-fold as compared to Matrigel invasion in vitro of the cells wherein the cells are treated with a control antibody (e.g., an antibody that does not target MUC16), In certain embodiments, Matrigel invasion is inhibited by about 1.25, 1.5, 1.75, 2, 3, 4, 5, 6, 7, 8, 9, or 10-fold as compared to Matrigel invasion in vitro of the cells wherein the cells are treated with a control antibody (e.g., an antibody that does not target MUC16).

Assays to determine the MUC16 anti-MUC16 antibody agent or antigen-binding fragment-mediated inhibition of Matrigel invasion are known to a person skilled in the art. For example, BD BioCoat™ Matrigel™ Invasion Inserts or Chambers (catalog #354480 in 24 well plate) and Control Inserts (catalog #354578 in 24 well plate) can be purchased from BD Biosciences, MA. Matrigel Invasion assay can be performed as per manufacturer's protocol. Briefly, the Matrigel chambers in 24 well plates (stored at −20° C.) and control inserts (stored at 4° C.) are allowed to come to room temperature. Both inserts are rehydrated with 0.5 ml of serum free medium in the insert as well as in the outside well of the 24 well plate, for 2 hours at 37° C. 5% CO₂ humidified incubator. Cultured SKOV3 cells are trypsinized and washed with culture medium. A million cells are separated into another centrifuge tube and washed 3 times with serum free medium. These cells are later adjusted to give 5,000 cells in 0.5 ml serum free medium. The medium in the rehydrated inserts are removed and the insert was transferred into a new 24 well plate containing 0.75 mL of 10% Fetal Bovine Serum (FBS) containing culture medium in the well which serves as a chemo attractant. Immediately. 0.5 mL of the cells (5,000 cells) in serum free medium is added to the insert. Proper care is taken to see that there is no air bubble is trapped in the insert and the outside well. The 24 well plate is incubated at 37° C.: 5% C02 humidified incubator for 48 hrs. After incubation, the non-invading cells are removed from the upper surface of the membrane by “scrubbing” by inserting a cotton tipped swab into Matrigel or control insert and gently applied pressure while moving the tip of the swab over the membrane surface. The scrubbing is repeated with a second swab moistened with medium. Then the inserts are stained in a new 24 well plate containing 0.5 mL of 0.5% crystal violet stain in distilled water for 30 minutes. Following staining the inserts are rinsed in 3 beakers of distilled water to remove excess stain. The inserts are air dried for in a new 24 well plate. The invaded cells are hand counted under an inverted microscope at 200× magnification. Several fields of triplicate membranes were counted and recorded in the figure.

In certain embodiments, an anti-MUC16 antibody agent or an antigen-binding fragment thereof described herein is capable of inhibiting or reducing metastasis, inhibiting tumor growth or inducing tumor regression in mouse model studies. For example, tumor cell lines can be introduced into athymic nude mice, and the athymic mice can be administered an anti-MUC16 antibody agent or an antigen-binding fragment thereof described herein one or more times, and tumor progression of the injected tumor cells can be monitored over a period of weeks and/or months. In some cases, administration of an anti-MUC16 antibody agent or an antigen-binding fragment thereof to the athymic nude mice can occur prior to introduction of the tumor cell lines. In a certain embodiment, SKOV3 cells expressing MUC16 c114 are utilized for the mouse xenograft models described herein.

In some embodiments, an anti-MUC16 antibody agent or an antigen-binding fragment thereof described herein inhibits tumor growth or induce tumor regression in a mouse model by at least about 5%, 10%, 15%, 20%, 25%, 30%, 35%, 40%, 45%, 50%, 55%, 60%, 65%, 70%, 75%, 80%, 85%, 90%, 95%, 98%, 99%, or 100% as assessed by methods described herein or known to one of skill in the art, as compared to mock-treated mice. In some embodiments, an anti-MUC16 antibody agent or an antigen-binding fragment thereof described herein inhibits tumor growth or induce tumor regression in a mouse model by at least about 25% or 35%), optionally to about 75%, as assessed by methods described herein or known to one of skill in the art, as compared to mock-treated mice. In some embodiments, an anti-MUC16 antibody agent or an antigen-binding fragment thereof described herein inhibit tumor growth or induce tumor regression in a mouse model by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art, as compared to mock-treated mice. Mock-treated mice can, for example, be treated with phosphate buffered saline or a control (e.g., anti-IgG antibody).

Determining tumor growth inhibition or tumor regression can be assessed, for example, by monitoring tumor size over a period of time, such as by physical measurement of palpable tumors, or other visual detection methods. For example, tumor cell lines can be generated to recombinantly express a visualization agent, such as green fluorescent protein (GFP) or luciferase, then in vivo visualization of GFP can be carried out by microscopy, and in vivo visualization of luciferase can be carried out by administering luciferase substrate to the xenograft mice and detecting luminescent due to the luciferase enzyme processing the luciferase substrate. The degree or level of detection of GFP or luciferase correlates to the size of the tumor in the xenograft mice.

In certain embodiments, an anti-MUC16 antibody agent or an antigen-binding fragment thereof described herein can increase survival of animals in tumor xenograft models as compared to mock-treated mice. In some embodiments, an anti-MUC16 antibody agent or an antigen-binding fragment thereof described herein increases survival of mice in tumor xenograft models by at least about 5%, 10%, 15%, 20%, 25%, 30%, 35%, 40%, 45%, 50% 55%, 60%, 65%, 70%, 75%, 80%, 85%, 90%, 95%, 98%, or 99% as assessed by methods described herein or known to one of skill in the art, as compared to mock-treated mice. In some embodiments, an anti-MUC16 antibody agent or an antigen-binding fragment thereof described herein increases survival of mice in tumor xenograft models by at least about 25% or 35%, optionally to about 75%), as assessed by methods described herein or known to one of skill in the art, as compared to mock-treated mice in tumor xenograft models. In some embodiments, an anti-MUC16 antibody agent or an antigen-binding fragment thereof described herein increases survival of mice in tumor xenograft models by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold. 90 fold, or 100 fold as assessed by methods described herein or known to one of skill in the art, as compared to mock-treated mice in tumor xenograft models. Survival can, for example, be determined by plotting a survival curve of number of surviving mice against time (e.g., days of weeks) after tumor cell line injection. Mock-treated mice can, for example, be treated with phosphate buffered saline or a control (e.g., anti-IgG antibody).

In certain embodiments, an anti-MUC16 antibody agent or an antigen-binding fragment thereof described herein is internalized into a cell expressing a MUC16 polypeptide upon contacting the cell with the anti-MUC16 antibody agent or an antigen-binding fragment thereof. “Internalized” or “internalization,” when in reference to a molecule that is internalized by a cell, refers to passage of the molecule that is in contact with the extracellular surface of a cell membrane across the cell membrane to the intracellular surface of the cell membrane and/or into the cell cytoplasm. In certain embodiments, the cells recombinantly expressing glycosylated MUC16 c114 are SKOV3 cells. In certain embodiments, the glycosylated form of MUC16 c114 is N-glycosylated, e.g., at Asn1, Asn24, and Asn30 of SEQ ID NO: 25 (also referred to as Asn1777, Asn1800, and Asn1806, respectively, in Yin and Lloyd (2001) J Biol Chem 276:27371-27375). In certain embodiments, the glycosylation comprises N-linked chitobiose. In certain embodiments, the glycosylation consists of an N-linked chitobiose.

Assays to determine internalization of an anti-MUC16 antibody agent or an antigen-binding fragment thereof described herein to a cell, such as, for example, using radiolabeled antibodies, are known to a person skilled in the art. For example, internalization of 89Zr-labeled antibody can be investigated on SKOV3 cells expressing MUC16 c114. Briefly, approximately 1×10⁵ cells are seeded in a 12-well plate and incubated overnight at 37° C. 5% CO₂ incubator. A volume of radiolabeled protein is added to each well and the plates are incubated at 37° C. and 4° C. for 1, 5, 12, and 24 hours. Following each incubation period, the medium is collected and the cells are rinsed with 1 mL of phosphate buffered saline (PBS). Surface-bound activity is collected by washing the cells in 1 ml of 100 mM acetic acid with 100 mM glycine (1:1, pH 3.5) at 4° C. The adherent cells are then lysed with 1 mL of 1 M NaOH. Each wash is collected and counted for activity. The ratio of activity of the final wash to the total activity of all the washes is used to determine the % internalized. In certain embodiments, the assay is performed at 37° C. In certain embodiments, the anti-MUC16 antibody agent or an antigen-binding fragment thereof is internalized in at least 1, 2, 3, 5, 6, 7, 8, 9, or 10 percent of cells incubated with the anti-MUC16 antibody agent or an antigen-binding fragment thereof. In certain embodiments, the anti-MUC16 antibody agent or an antigen-binding fragment thereof is internalized in about 1, 2, 3, 5, 6, 7, 8, 9, or 10 percent of cells incubated with the anti-MUC16 antibody agent or an antigen-binding fragment thereof. In certain embodiments, the anti-MUC16 antibody agent or an antigen-binding fragment thereof is internalized within 1, 2, 3, 4, 8, 12, 16, 20, or 24 hours of contacting the cells with the anti-MUC16 antibody agent or an antigen-binding fragment thereof.

### Nucleic Acids

Nucleic acid molecules encoding the anti-MUC16 antibody agents or an antigen-binding fragment thereof (such as anti-MUC16 antibodies, e.g., full-length anti-MUC16 antibodies) are also contemplated. In some embodiments, there is provided a nucleic acid (or a set of nucleic acids) encoding a full-length anti-MUC16 antibody, including any of the full-length anti-MUC16 antibodies described herein, or an antigen-binding fragment thereof. In some embodiments, the nucleic acid (or a set of nucleic acids) encoding the anti-MUC16 antibody agent described herein may further comprises a nucleic acid sequence encoding a peptide tag (such as protein purification tag, e.g., His-tag. HA tag).

Also contemplated here are isolated host cells comprising an anti-MUC16 antibody agent, an isolated nucleic acid encoding the polypeptide components of the anti-MUC16 antibody agent, or a vector comprising a nucleic acid encoding the polypeptide components of the anti-MUC16 antibody agent described herein.

The present application also includes variants to these nucleic acid sequences. For example, the variants include nucleotide sequences that hybridize to the nucleic acid sequences encoding the anti-MUC16 antibody agents (such as anti-MUC16 antibodies, e.g., full-length anti-MUC16 antibodies), antigen-binding fragments thereof, or anti-MUC16 antibody moieties of the present application under at least moderately stringent hybridization conditions.

The present invention also provides vectors in which a nucleic acid of the present invention is inserted.

In brief summary, the expression of an anti-MUC16 antibody agent (e.g., full-length anti-MUC16 antibody) or an antigen-binding fragment thereof by a natural or synthetic nucleic acid encoding the anti-MUC16 antibody agent can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584: WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter. MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the anti-MUC16 antibody agent is inducible. In some embodiments, a nucleic acid sequence encoding the anti-MUC16 antibody agent is operably linked to an inducible promoter, including any inducible promoter described herein.

### Inducible Promoters

The use of an inducible promoter provides a molecular switch capable of turning on expression of the polynucleotide sequence which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. Proc. Natl. Acad. Sci. USA 90:5603-5607 (1993)), synthetic ligand-regulated elements (see, e.g., Spencer, D. M. et al 1993) Science 262:1019-1024) and ionizing radiation-regulated elements (e.g., see Manome, Y. et al., Biochemistry 32:10607-10613 (1993); Datta, R. et al., Proc. Natl. Acad. Sei. USA 89:1014-10153 (1992)). Further exemplary inducible promoter systems for use in in vitro or in vivo mammalian systems are reviewed in Gingrich et al., Annual Rev. Neurosci 21:377-405 (1998). In some embodiments, the inducible promoter system for use to express the anti-MUC16 antibody agent is the Tet system. In some embodiments, the inducible promoter system for use to express the anti-MUC16 antibody agent is the lac repressor system from E. coli.

An exemplary inducible promoter system for use in the present invention is the Tet system. Such systems are based on the Tet system described by Gossen et al., (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Ter-Off” and “Tet-On” systems. In the Tet-Off system, transcription is inactive in the presence of Tc or Dox. In that system, a tetracycline-controlled transactivator protein (ITA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, ITA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli (See Brown et al., Cell 49:603-612 (1987). The lac repressor system functions by regulating transcription of a polynucleotide of interest operably linked to a promoter comprising the lac operator (JacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase. β-galactosidase, chloramphenicol acetyl transferase, secreted alkaline phosphatase, or the green fluorescent protein gene (e.g., Ui-Tel et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a full-length anti-MUC16 antibody according to any of the full-length anti-MUC16 antibodies described herein. In some embodiments, the nucleic acid comprises one or more nucleic acid sequences encoding the heavy and light chains of the full-length anti-MUC16 antibody. In some embodiments, each of the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example. Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Preparation of Anti-MUC16 Antibody Agents and Anti-MUC16 Antibody Moieties

In some embodiments, the anti-MUC16 antibody agent is a monoclonal antibody or derived from a monoclonal antibody. In some embodiments, the anti-MUC16 antibody agent comprises V_{H} and V_{L} domains, or variants thereof, from the monoclonal antibody. In some embodiments, the anti-MUC16 antibody agent further comprises C_{H}1 and C_{L} domains, or variants thereof, from the monoclonal antibody. Monoclonal antibodies can be prepared, e.g., using known methods in the art, including hybridoma methods, phage display methods, or using recombinant DNA methods. Additionally, exemplary phage display methods are described herein and in the Examples below.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are marine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Ana!. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the anti-MUC16 antibody agents described herein, the anti-MUC16 antibody agent comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352:624-628 (1991); Marks et al., J. Mol. Biol. 222:581-597 (1992); Marks and Bradbury, Methods in Molecular Biology 248:161-175 (Lo, ed., Human Press, Totowa, N.J., 2003): Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284 (1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann Rev. Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The anti-MUC16 antibody agents can be prepared using phage display to screen libraries for anti-MUC16 antibody moieties specific to the target MUC16 (e.g., nMUC16). The library can be a human scFv phage display library having a diversity of at least one ×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see. e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target MUC16 (e.g., nMUC16) with high affinity can be selected by iterative binding of phage to the target MUC16, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XLI-Blue, for expression and purification. In an example of cell panning, HEK293 cells over-expressing MUC16 on cell surface are mixed with the phage library, after which the cells are collected and the bound clones are eluted and used to infect an appropriate host cell for expression and purification (all see Examples). The panning can be performed for multiple (such as about any of 2, 3, 4, 5, 6 or more) rounds with solution panning, cell panning, or a combination of both, to enrich for phage clones binding specifically to the target MUC16. Enriched phage clones can be tested for specific binding to the target MUC16 by any methods known in the art, including for example ELISA and FACS.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). Hybridoma cells as described above or MUC16-specific phage clones of the invention can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody agent of the invention, or can be substituted for the variable domains of one antigen-combining site of an antibody agent of the invention to create a chimeric bivalent antibody agent.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism.

### Human and Humanized Antibodies

The anti-MUC16 antibody agents (e.g., full-length anti-MUC16 antibodies) or an antigen-binding fragment thereof can be humanized antibody agents or human antibody agents. Humanized forms of non-human (e.g., murine) antibody moieties are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin, and all or substantially all of the FR regions are those of a human immunoglobulin consensus sequence.

Generally, a humanized antibody agent has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321:522-525 (1986): Riechmann et al., Nature, 332:323-327 (1988): Verhoeven et al., Science, 239:1534-1536 (1988)), by substituting rodent CDRs or CDR sequences for the corresponding sequences of a human antibody. Accordingly, such “humanized” antibody moieties are antibody moieties (U.S. U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993): U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10:779-783 (1992): Lonberg et al., Nature, 368:856-859 (1994); Morrison, Nature, 368:812-813 (1994); Fishwild et al., Nature Biotechnology, 14:845-851 (1996); Neuberger, Nature Biotechnology, 14:826 (1996); Lonberg and Huszar, Intern. Rev. Immunol., 13:65-93 (1995)

Human antibody agents may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147 (1): 86-95 (1991).

### Anti-MUC16 Antibody Agent Variants

In some embodiments, amino acid sequence variants of the anti-MUC16 antibody agents (e.g., full-length anti-MUC16 antibody) or an antigen-binding fragment thereof provided herein are contemplated. For example, it may be desirable to improve the binding affinity and/or other biological properties of the antibody agent. Amino acid sequence variants of an antibody agent may be prepared by introducing appropriate modifications into the nucleotide sequence encoding the antibody agent, or by peptide synthesis. Such modifications include, for example, deletions from, and/or insertions into and/or substitutions of residues within the amino acid sequences of the antibody agent. Any combination of deletion, insertion, and substitution can be made to arrive at the final construct, provided that the final construct possesses the desired characteristics, e.g., antigen-binding.

In some embodiments, anti-MUC16 antibody agent variants having one or more amino acid substitutions are provided. Sites of interest for substitutional mutagenesis include the HVRs and FRs. Amino acid substitutions may be introduced into an antibody agent of interest and the products screened for a desired activity, e.g., retained/improved antigen binding, decreased immunogenicity, or improved ADCC or CDC.

Conservative substitutions are shown in Table 4 below.

### Table 4: Conservative Substitutions

TABLE 4
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties: hydrophobic: Norleucine, Met, Ala, Val, Leu, Ile; neutral hydrophilic: Cys, Ser, Thr, Asn. Gln; acidic: Asp, Glu; basic: His, Lys, Arg; residues that influence chain orientation: Gly, Pro; and aromatic: Trp. Tyr, Phe, Non-conservative substitutions involves exchanging a member of one of these classes for another class.

An exemplary substitutional variant is an affinity matured antibody agent, which may be conveniently generated, e.g., using phage display-based affinity maturation techniques. Briefly, one or more CDR residues are mutated and the variant antibody moieties displayed on phage and screened for a particular biological activity (e.g., binding affinity). Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} or V_{L} being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001).)

In some embodiments of affinity maturation, diversity is introduced into the variable genes chosen for maturation by any of a variety of methods (e.g. error-prone PCR, chain shuffling, or oligonucleotide-directed mutagenesis). A secondary library is then created. The library is then screened to identify any antibody agent variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody agent to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant VH and VL sequences provided above, each HVR either is unaltered, or contains no more than one, two or three amino acid substitutions.

A useful method for identification of residues or regions of an antibody agent that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody agent with antigen is affected. Further substitutions may be introduced at the amino acid locations demonstrating functional sensitivity to the initial substitutions. Alternatively, or additionally, a crystal structure of an antigen-antibody agent complex can be determined to identify contact points between the antibody agent and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody agent with an N-terminal methionyl residue. Other insertional variants of the antibody agent molecule include the fusion to the N- or C-terminus of the antibody agent to an enzyme (e.g. for ADEPT) or a polypeptide which increases the serum half-life of the antibody agent.

### Fe Region Variants

In some embodiments, one or more amino acid modifications may be introduced into the Fc region of an antibody agent (e.g., a full-length anti-MUC16 antibody or anti-MUC16 Fc fusion protein) provided herein, thereby generating an Fe region variant. In some embodiments, the Fc region variant has enhanced ADCC effector function, often related to binding to Fc receptors (FcRs). In some embodiments, the Fe region variant has decreased ADCC effector function. There are many examples of changes or mutations to Fc sequences that can alter effector function. For example, WO 00/42072 and Shields et al., J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-MUC16 antibody). The typical ADCC involves activation of NK cells by antibodies. An NK cell expresses CD16 which is an Fe receptor. This receptor recognizes, and binds to, the Fc portion of an antibody bound to the surface of a target cell. The most common Fe receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the invention contemplates an anti-MUC16 antibody agent variant (such as a full-length anti-MUC16 antibody variant) comprising an Fc region that possesses some but not all effector functions, which makes it a desirable candidate for applications in which the half-life of the anti-MUC16 antibody agent in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody agent lacks FeγR binding (hence likely lacking ADCC activity), but retains FeRn binding ability. The primary cells for mediating ADCC, NK cells, express FcγRIII only, whereas monocytes express FeγRI, FeγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, L. et al., Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998). C1q binding assays may also be carried out to confirm that the antibody agent is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg. M. S. et al., Blood 101:1045-1052 (2003); and Cragg. M. S. and M. J. Glennie, Blood 103:2738-2743 (2004). FcRn binding and in vivo clearance/half-life determinations can also be performed using methods known in the art (see, e.g., Petkova, S. B. et al., Int'l. Immunol. 18 (12): 1759-1769 (2006)).

Antibodies with reduced effector function include those with substitution of one or more of Fc region residues 238, 265, 269, 270, 297, 327 and 329 (U.S. Pat. No. 6,737,056). Such Fe mutants include Fc mutants with substitutions at two or more of amino acid positions 265, 269, 270, 297 and 327, including the so-called “DANA” Fe mutant with substitution of residues 265 and 297 to alanine (U.S. Pat. No. 7,332,581).

Certain antibody agent variants with improved or diminished binding to FcRs are described. (See, e.g., U.S. Pat. No. 6,737,056; WO 2004/056312, and Shields et al., J. Biol. Chem. 9 (2): 6591-6604 (2001).)

In some embodiments, there is provided an anti-MUC16 antibody agent (such as a full-length anti-MUC16 antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fe region (EU numbering of residues). In some embodiments, the anti-MUC16 antibody agent (e.g., full-length anti-MUC16 antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164:4178-4184 (2000).

In some embodiments, there is provided an anti-MUC16 antibody agent (such as a full-length anti-MUC16 antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FeRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988): U.S. Pat. Nos. 5,648,260; 5,624,821; and WO 94/29351 concerning other examples of Fc region variants.

Anti-MUC16 antibody agents (such as full-length anti-MUC16 antibodies) comprising any of the Fc variants described herein, or combinations thereof, are contemplated.

### Glycosylation Variants

In some embodiments, an anti-MUC16 antibody agent (such as a full-length anti-MUC16 antibody) or an antigen-binding fragment thereof provided herein is altered to increase or decrease the extent to which the anti-MUC16 antibody agent is glycosylated. Addition or deletion of glycosylation sites to an anti-MUC16 antibody agent may be conveniently accomplished by altering the amino acid sequence of the anti-MUC16 antibody agent or polypeptide portion thereof such that one or more glycosylation sites is created or removed.

Where the anti-MUC16 antibody agent or an antigen-binding fragment thereof comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-MUC16 antibody agent of the invention may be made in order to create anti-MUC16 antibody agent variants with certain improved properties.

The N-glycans attached to the CH2 domain of Fe is heterogeneous. Antibodies or Fc fusion proteins generated in CHO cells are fucosylated by fucosyltransferase activity. See Shoji-Hosaka et al., J. Biochem. 140:777-83 (2006). Normally, a small percentage of naturally occurring afucosylated IgGs may be detected in human serum. N-glycosylation of the Fe is important for binding to FeγR; and afucosylation of the N-glycan increases Fc's binding capacity to FcγRIIIa. Increased FcγRIIIa binding can enhance ADCC, which can be advantageous in certain antibody agent therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-MUC16 antibody agent (such as a full-length anti-MUC16 antibody) variants are provided comprising an Fc region wherein a carbohydrate structure attached to the Fc region has reduced fucose or lacks fucose, which may improve ADCC function. Specifically, anti-MUC16 antibody agents are contemplated herein that have reduced fucose relative to the amount of fucose on the same anti-MUC16 antibody agent produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-MUC16 antibody agent is one wherein less than about 50%, 40%, 30%, 20%, 10%, or 5% of the N-linked glycans thereon comprise fucose. For example, the amount of fucose in such an anti-MUC16 antibody agent may be from 1% to 80%, from 1% to 65%, from 5% to 65% or from 20% to 40%. In some embodiments, the anti-MUC16 antibody agent is one wherein none of the N-linked glycans thereon comprise fucose, i.e., wherein the anti-MUC16 antibody agent is completely without fucose, or has no fucose or is afucosylated. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See. e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody agent variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742: WO2002/031140; Okazaki et al., J. Mol. Biol, 336:1239-1249 (2004); Yamane-Ohnuki et al., Biotech. Bioeng. 87:614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8, knockout CHO cells (see, e.g., Yamane-Ohmuki et al., Biotech. Bioeng, 87:614 (2004); Kanda, Y. et al., Biotechnol. Bioeng. 94(4):680-688 (2006); and WO2003/085107).

Anti-MUC16 antibody agent (such as a full-length anti-MUC16 antibody) variants are further provided with bisected oligosaccharides, e.g., in which a biantennary oligosaccharide attached to the Fc region of the anti-MUC16 antibody agent is bisected by GlcNAc. Such anti-MUC16 antibody agent (such as a full-length anti-MUC16 antibody) variants may have reduced fucosylation and/or improved ADCC function. Examples of such antibody agent variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-MUC16 antibody agent (such as full-length anti-MUC16 antibody) variants with at least one galactose residue in the oligosaccharide attached to the Fc region are also provided. Such anti-MUC16 antibody agent variants may have improved CDC function. Such antibody agent variants are described, e.g., in WO 1997/30087 (Patel et al.); WO 1998/58964 (Raju, S.); and WO 1999/22764 (Raju, S.).

In some embodiments, the anti-MUC16 antibody agent (such as a full-length anti-MUC16 antibody) variants comprising an Fc region are capable of binding to an FcγRIII In some embodiments, the anti-MUC16 antibody agent (such as a full-length anti-MUC16 antibody) variants comprising an Fc region have ADCC activity in the presence of human effector cells (e.g., T cell) or have increased ADCC activity in the presence of human effector cells compared to the otherwise same anti-MUC16 antibody agent (such as a full-length anti-MUC16 antibody) comprising a human wild-type IgG1Fc region.

### Cysteine Engineered Variants

In some embodiments, it may be desirable to create cysteine engineered anti-MUC16 antibody agents (such as a full-length anti-MUC16 antibody) or an antigen-binding fragment thereof in which one or more amino acid residues are substituted with cysteine residues. In some embodiments, the substituted residues occur at accessible sites of the anti-MUC16 antibody agent or an antigen-binding fragment thereof. By substituting those residues with cysteine, reactive thiol groups are thereby positioned at accessible sites of the anti-MUC16 antibody agent and may be used to conjugate the anti-MUC16 antibody agent to other moieties, such as drug moieties or linker-drug moieties, to create an anti-MUC16 immunoconjugate, as described further herein. Cysteine engineered anti-MUC16 antibody agents (such as anti-MUC16 antibodies, e.g., full-length anti-MUC16 antibodies) may be generated as described, e.g., in U.S. Pat. No. 7,521,541.

### Derivatives

In some embodiments, an anti-MUC16 antibody agent (such as a full-length anti-MUC16 antibody) or an antigen-binding fragment thereof provided herein may be further modified to contain additional non-proteinaceous moieties that are known in the art and readily available. The moieties suitable for derivatization of the anti-MUC16 antibody agent include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-MUC16 antibody agent may vary, and if more than one polymer are attached, they can be the same or different molecules. In general, the number and/or type of polymers used for derivatization can be determined based on considerations including, but not limited to, the particular properties or functions of the anti-MUC16 antibody agent to be improved, whether the anti-MUC16 antibody agent derivative will be used in a therapy under defined conditions, etc.

In some embodiments, conjugates of an anti-MUC16 antibody agent (such as a full-length anti-MUC16 antibody) or an antigen-binding fragment thereof and nonproteinaceous moiety that may be selectively heated by exposure to radiation are provided. In some embodiments, the nonproteinaceous moiety is a carbon nanotube (Kam et al., Proc. Natl. Acad. Sci. USA 102:11600-11605 (2005). The radiation may be of any wavelength, and includes, but is not limited to, wavelengths that do not harm ordinary cells, but which heat the nonproteinaceous moiety to a temperature at which cells proximal to the anti-MUC16 antibody agent-nonproteinaceous moiety are killed.

### Antibody Conjugates

In certain embodiments, provided herein are anti-MUC16 antibody agent or antigen-binding fragments thereof conjugates, wherein said anti-MUC16 antibody agent or antigen-binding fragments thereof is conjugated to one or more agents, e.g., an imaging agent or a cytotoxic agent. Also provided herein are bispecific antibody conjugates, wherein said bispecific antibody is conjugated to one or more agent, e.g., an imaging agent or a cytotoxic agent. Also provided herein are antibody heavy chain conjugates, wherein said antibody heavy chain is conjugated to one or more agent, e.g., an imaging agent or a cytotoxic agent. Also provided herein are antibody light chain conjugates, wherein said antibody light chain is conjugated to one or more agent, e.g., an imaging agent or a cytotoxic agent. Also provided herein are fusion protein conjugates, wherein said fusion protein is conjugated to an agent, e.g., an imaging agent or a cytotoxic agent. In certain embodiments, the agent is conjugated covalently or non-covalently.

In certain embodiments, the imaging agent is a detectable label, such as, a chromogenic, enzymatic, radioisotopic, isotopic, fluorescent, toxic, chemiluminescent, nuclear magnetic resonance contrast agent or other label.

Non-limiting examples of suitable chromogenic labels include diaminobenzidine and 4-hydroxyazo-benzene-2-carboxylic acid.

Non-limiting examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Suitable radioisotopes are well known to those skilled in the art and include beta-emitters, gamma-emitters, positron-emitters, and x-ray emitters. Non-limiting examples of suitable radioisotopic labels include ³H, ¹⁸F, ¹¹¹In, ¹²⁵I, ¹³¹I, ³²P, ³³P, ³S, ¹C, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Cl, ²¹¹At, ²¹²Pb, ⁴⁷Sc, ²²³Ra, ²²³Ra, ⁸⁹Zr, ¹⁷⁷Lu, and ¹⁰⁹Pd. In certain embodiments, ¹¹¹In is a preferred isotope for in vivo imaging as it avoids the problem of dehalogenation of ¹²⁵I or ¹³¹I-labeled anti-MUC16 antibody agents or antigen-binding fragments thereof in the liver. In addition, ¹¹¹In has a more favorable gamma emission energy for imaging (Perkins et al, Eur. J. Nucl. Med. 70:296-301 (1985); Carasquillo et ah, J. Nucl. Med. 25:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Non-limiting examples of suitable non-radioactive isotopic labels include 157Gd, ⁵³ Min, ¹⁶²Dy, ⁵²Tr, and ⁵⁶Fe.

Non-limiting examples of suitable fluorescent labels include a ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label.

Non-limiting examples of chemiluminescent labels include a luminol label, an isoluminol label, an aromatic acridinium ester label, an imidazole label, an acridinium salt label, an oxalate ester label, a luciferin label, a luciferase label, and an aequorin label.

Non-limiting examples of nuclear magnetic resonance contrasting agents include heavy metal nuclei such as Od, Mn, and iron.

Techniques known to one of ordinary skill in the art for conjugating the above-described labels to said anti-MUC16 antibody agents or antigen-binding fragments thereof, bispecific antibodies, antibody heavy chains, antibody light chains, and fusion proteins are described in, for example, Kennedy et at., Clin. CMm. Acta 70: 1-31 (1976), and Schurs et al. Clin. CMm. Acta 81: 1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

Nonlimiting examples of cytotoxic agents include a cytostatic or cytocidal agent, a radioactive metal ion, e.g., alpha-emitters, and toxins, e.g., pseudomonas exotoxin, abrin, cholera toxin, ricin A, and diphtheria toxin.

In certain embodiments, the agent is a diagnostic agent. A diagnostic agent is an agent useful in diagnosing or detecting a disease by locating the cells containing the antigen. Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. Preferably, the diagnostic agents are selected from the group consisting of radioisotopes, enhancing agents for use in magnetic resonance imaging, and fluorescent compounds. In order to load an anti-MUC16 antibody agent or antigen-binding fragment thereof with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, for example, ethylenediaminetetraacetic acid (EDTA), diethylenetriaminepentaacetic acid (DTP A), porphyrins, polyamines, crown ethers, bis-thiosemicarbazones, polyoximes, and like groups known to be useful for this purpose. Chelates are coupled to the antibodies using standard chemistries. The chelate is normally linked to the antibody by a group which enables formation of a bond to the molecule with minimal loss of immunoreactivity and minimal aggregation and/or internal cross-linking other, more unusual, methods and reagents for conjugating chelates to antibodies are disclosed in U.S. Pat. No. 4,824,659 to Hawthorne, entitled “Antibody Conjugates,” issued Apr. 25, 1989, the disclosure of which is incorporated herein in its entirety by reference. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MRI, when used along with an anti-MUC16 antibody agent or antigen-binding fragment thereof provided herein.

Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates such as macrocyclic polyethers, which are of interest for stably binding nuclides, such as ²²³Ra for RAIT are encompassed herein.

### Pharmaceutical Compositions

Also provided herein are compositions (such as pharmaceutical compositions, also referred to herein as formulations) comprising an anti-MUC16 antibody agent (such as a full-length anti-MUC16 antibody) or an antigen-binding fragment thereof, nucleic acid encoding the antibody agent, vector comprising the nucleic acid encoding the antibody agent, or host cell comprising the nucleic acid or vector. In some embodiments, there is provided a pharmaceutical composition comprising an anti-MUC16 antibody agent and optionally a pharmaceutically acceptable carrier.

Suitable formulations of the anti-MUC16 antibody agents (such as anti-MUC16 antibodies, e.g., full-length anti-MUC16 antibodies) or an antigen-binding fragment thereof are obtained by mixing an anti-MUC16 antibody agent having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-MUC16 antibody agents of this invention into cells.

The formulation herein may also contain one or more active compounds in addition to the anti-MUC16 antibody agent (such as a full-length anti-MUC16 antibody) or an antigen-binding fragment thereof as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-MUC16 antibody agent or an antigen-binding fragment thereof. Such molecules are suitably present in combination in amounts that are effective for the purpose intended. The effective amount of such other agents depends on the amount of anti-MUC16 antibody agent present in the formulation, the type of disease or disorder or treatment, and other factors discussed above. These are generally used in the same dosages and with administration routes as described herein or about from 1 to 99% of the heretofore employed dosages.

The anti-MUC16 antibody agents (such as anti-MUC16 antibodies, e.g., full-length anti-MUC16 antibodies) or an antigen-binding fragment thereof may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared.

Sustained-release preparations of the anti-MUC16 antibody agents (such as anti-MUC16 antibodies, e.g., full-length anti-MUC16 antibodies) or an antigen-binding fragment thereof can be prepared. Suitable examples of sustained-release preparations include semipermeable matrices of solid hydrophobic polymers containing the antibody agent (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D (−)-3-hydroxybutyric acid. While polymers such as ethylene-vinyl acetate and lactic acid-glycolic acid enable release of molecules for over 100 days, certain hydro gels release proteins for shorter time periods. When encapsulated antibody agents remain in the body for a long time, they can denature or aggregate as a result of exposure to moisture at 37° C., resulting in a loss of biological activity and possible changes in immunogenicity. Rational strategies can be devised for stabilization of anti-MUC16 antibody agents depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-MUC16 antibody agent (such as a full-length anti-MUC16 antibody) or an antigen-binding fragment thereof is formulated in a buffer comprising a citrate, NaCl, acetate, succinate, glycine, polysorbate 80 (Tween 80), or any combination of the foregoing. In some embodiments, the anti-MUC16 antibody agent or an antigen-binding fragment thereof is formulated in a buffer comprising about 100 mM to about 150 mM glycine. In some embodiments, the anti-MUC16 antibody agent or an antigen-binding fragment thereof is formulated in a buffer comprising about 50 mM to about 100 mM NaCl. In some embodiments, the anti-MUC16 antibody agent or an antigen-binding fragment thereof is formulated in a buffer comprising about 10 mM to about 50 mM acetate. In some embodiments, the anti-MUC16 antibody agent or an antigen-binding fragment thereof is formulated in a buffer comprising about 10 mM to about 50 mM succinate. In some embodiments, the anti-MUC16 antibody agent or an antigen-binding fragment thereof is formulated in a buffer comprising about 0.005% to about 0.02% polysorbate 80. In some embodiments, the anti-MUC16 antibody agent or an antigen-binding fragment thereof is formulated in a buffer having a pH between about 5.1 and 5.6. In some embodiments, the anti-MUC16 antibody agent or an antigen-binding fragment thereof is formulated in a buffer comprising 10 mM citrate, 100 mM NaCl, 100 mM glycine, and 0.01% polysorbate 80, wherein the formulation is at pH 5.5.

The formulations to be used for in vivo administration must be sterile. This is readily accomplished by, e.g., filtration through sterile filtration membranes.

### Methods of Treatment Using Anti-MUC16 Antibody Agents

In certain embodiments, provided herein are methods for treating a cancer in a subject, in particular, a MUC16-positive cancer in a subject, comprising administering to the subject in need thereof a therapeutically effective amount of anti-MUC16 antibody agent or an antigen-binding fragment thereof. In some embodiments, the anti-MUC16 antibody agent or antigen-binding fragment thereof is administered at a therapeutically effective dose, such as a dose described herein. In some embodiments, the anti-MUC16 antibody agent or antigen-binding fragment thereof is administered according to a method as described herein. In some embodiments, the anti-MUC16 antibody agent or antigen-binding fragment thereof is administered in combination with one or more additional pharmaceutically active agents.

For use of an anti-MUC16 antibody agent or antigen-binding fragment thereof in a subject of a particular species, an anti-MUC16 antibody agent or antigen-binding fragment thereof is used that binds to MUC16 of that particular species. For example, to treat a human, an anti-MUC16 antibody agent or antigen-binding fragment thereof is used that binds to human MUC16. In some embodiments, the anti-MUC16 antibody agent or antigen-binding fragment thereof is an immunoglobulin.

In addition, for use of an anti-MUC16 antibody agent or antigen-binding fragment thereof in a subject of a particular species, the anti-MUC16 antibody agent, preferably, the constant region of an anti-MUC16 antibody agent or antigen-binding fragment thereof, is derived from that particular species. For example, to treat a human, an anti-MUC16 antibody agent or antigen-binding fragment thereof can comprise an anti-MUC16 antibody agent or antigen-binding fragment thereof that is an immunoglobulin, wherein the immunoglobulin comprises a human constant region. In some embodiments, the subject is a human.

In some embodiments, the MUC16-positive cancer is ovarian cancer, lung cancer, pancreatic cancer, breast cancer, fallopian tube cancer, uterine (e.g., endometrial) cancer, primary peritoneum cancer or cancer of any other tissue that expresses the MUC16 receptor.

In some embodiments, treatment can be to achieve beneficial or desired clinical results including, but not limited to, alleviation of a symptom, diminishment of extent of a disease, stabilizing (i.e., not worsening) of state of a disease, delay or slowing of disease progression, amelioration or palliation of the disease state, and remission (whether partial or total), whether detectable or undetectable. In a specific embodiment, “treatment” can also be to prolong survival as compared to expected survival if not receiving treatment. In some embodiments, the administration of an anti-MUC16 antibody agent or an antigen-binding fragment thereof described herein, or a pharmaceutical composition described herein to a, subject with cancer (e.g., ovarian cancer, lung cancer, pancreatic cancer, breast cancer, fallopian tube cancer, uterine (e.g., endometrial) cancer, or primary peritoneum cancer, or cancer of any other tissue that expresses the MUC16 receptor) achieves at least one, two, three, four or more of the following effects: (i) the reduction or amelioration of the severity of one or more symptoms of cancer; (ii) the reduction in the duration of one or more symptoms associated with cancer; (iii) the prevention in the recurrence of a symptom associated with cancer; (iv) the reduction in hospitalization of a subject; (v) a reduction in hospitalization length; (vi) the increase in the survival of a subject, (vii) the enhancement or improvement of the therapeutic effect of another therapy; (viii) the inhibition of the development or onset of one or more symptoms associated with cancer; (ix) the reduction in the number of symptoms associated with cancer; (x) improvement in quality of life as assessed by methods well known in the art; (x) inhibition of the recurrence of a tumor; (xi) the regression of tumors and/or one or more symptoms associated therewith: (xii) the inhibition of the progression of tumors and/or one or more symptoms associated therewith; (xiii) a reduction in the growth of a tumor; (xiv) a decrease in tumor size (e.g., volume or diameter); (xv) a reduction in the formation of a newly formed tumor; (xvi) prevention, eradication, removal, or control of primary, regional and/or metastatic tumors; (xvii) a decrease in the number or size of metastases; (xviii) a reduction in mortality; (xix) an increase in relapse free survival: (xx) the size of the tumor is maintained and does not increase or increases by less than the increase of a tumor after administration of a standard therapy as measured by conventional methods available to one of skill in the art, such as magnetic resonance imaging (MRI), dynamic contrast-enhanced MRI (DCE-MRI), X-ray, and computed tomography (CT) scan, or a positron emission tomography (PET) scan; and/or (xxi) an increase in the length of remission in patients. Treatment can be to achieve one or more of the foregoing.

### Diagnostic Uses

In certain embodiments, anti-MUC16 antibody agents or antigen-binding fragments thereof described herein can be used for diagnostic purposes to detect, diagnose, or monitor a condition described herein (e.g., a condition involving MUC16-positive cancer cells). In certain embodiments, anti-MUC16 antibody agents or antigen-binding fragments thereof for use in diagnostic purposes are labeled.

In certain embodiments, provided herein are methods for the detection of a condition described herein comprising (a) assaying the expression of MUC16 or a fragment thereof in cells or a tissue sample of a subject using one or more anti-MUC16 antibody agents or antigen-binding fragments thereof described herein; and (b) comparing the level of MUC16 or the fragment thereof expression with a control level, for example, levels in normal tissue samples (e.g., from a subject not having a condition described herein, or from the same patient before onset of the condition), whereby an increase or decrease in the assayed level of MUC16 or the fragment thereof expression compared to the control level of MUC16 or the fragment thereof expression is indicative of a condition described herein.

Antibodies described herein can be used to assay the levels of MUC16 or a fragment thereof in a biological sample using classical immunohistological methods as described herein or as known to those of skill in the art (e.g., see Jalkanen et al., J. Cell. Biol. 101:976-985 (1985); and Jalkanen et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA), Suitable antibody assay labels are known in the art and include enzyme labels, such as, glucose oxidase: radioisotopes, such as iodine (¹²⁵I, 121I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. In some embodiments, the assay labels are conjugated to the anti-MUC16 antibody agents or antigen-binding fragment thereof provided herein for direct detection. In some embodiments, the assay labels are conjugated to a secondary antibody that binds to an anti-MUC16 antibody agents or antigen-binding fragment thereof provided herein. The secondary antibody type is selected according to the class of the primary antibody (e.g., IgG or IgM), the source host, and the kind of label which is preferred. In some embodiments, the secondary antibody is a class or isotype specific antibody (e.g., IgG, IgM, IgA, IgE or IgG). In some embodiments, the secondary antibody is a subclass specific antibody (e.g., IgG1, IgG2, IgG2, IgG4, IgA1, or IgA2), In some embodiments, the secondary antibody binds to one or more classes or subclasses of antibodies. In some embodiments, the secondary antibody binds to the heavy chain of the primary antibody. In some embodiments, the secondary antibody binds to the light chain of the primary antibody. In some embodiments, the secondary antibody binds to a kappa light chain of the primary antibody. In some embodiments, the secondary antibody binds to a lambda light chain of the primary antibody. In some embodiments, the secondary antibody is an anti-Fc or an anti-F(ab) or anti-(Fab′)2 fragment antibody. In some embodiments, the secondary antibody is a rabbit, mouse, goat, donkey or chicken antibody.

In certain embodiments, monitoring of a condition described herein (e.g., a MUC16-positive cancer), is carried out by repeating the method for diagnosing for a period of time after initial diagnosis.

Presence of the labeled molecule can be detected in the subject (I.e., in vivo) using methods known in the art for in vivo scanning. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

An anti-MUC16 antibody agent or antigen-binding fragment thereof as described herein, or composition containing, or cells expressing the antibodies, or antigen-binding fragments thereof, described herein may be delivered to a subject by a variety of routes. These include, but are not limited to, parenteral, intranasal, intratracheal, oral, intradermal, topical, intramuscular, intraperitoneal, transdermal, intravenous, intratumoral, conjunctival and subcutaneous routes. Pulmonary administration can also be employed, e.g., by use of an inhaler or nebulizer, and formulation with an aerosolizing agent for use as a spray. In one embodiment, an anti-MUC16 antibody agent or antigen-binding fragment thereof, or a composition described herein is administered parenterally to a subject. In some embodiments, said parenteral administration is intravenous, intramuscular, or subcutaneous.

The amount of an anti-MUC16 antibody agent or antigen-binding fragment thereof, or composition which will be effective in the treatment and/or prevention of a condition will depend on the nature of the disease, and can be determined by standard clinical techniques.

The precise dose to be employed in a composition will also depend on the route of administration, and the type of cancer, and should be decided according to the judgment of the practitioner and each subject's circumstances. For example, effective doses may also vary depending upon means of administration, target site, physiological state of the patient (including age, body weight and health), whether the patient is human or animal, other medications administered, or whether treatment is prophylactic or therapeutic. Treatment dosages are optimally titrated to optimize safety and efficacy.

In certain embodiments, an in vitro assay is employed to help identify optimal dosage ranges. Effective doses may be extrapolated from dose response curves derived from in vitro or animal model test systems.

For an anti-MUC16 antibody agent or an antigen-binding fragment thereof, the dosage may range from about 0.0001 to 100 mg/kg, and more usually 0.01 to 15 mg/kg, of the patient body weight. For example, dosages can be 1 mg/kg body weight, 10 mg/kg body weight, or within the range of 1-10 mg/kg or in other words. 70 mg or 700 mg or within the range of 70-700 mg, respectively, for a 70 kg patient. Generally, human antibodies have a longer half-life within the human body than antibodies from other species due to the immune response to the foreign polypeptides. Thus, lower dosages of human antibodies and less frequent administration is often possible.

In certain embodiments, such as in the administration of engineered cells expressing the antibodies or antigen-binding fragments thereof, or CARs, a subject is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In some embodiments, the dose of total cells and/or dose of individual sub-populations of cells is within a range of between at or about 104 and at or about 10° cells/kilograms (kg) body weight, such as between 1° and 10° cells/kg body weight, for example, at or about 1×10⁵ cells/kg, 1.5×10⁵ cells/kg, 2×10⁵ cells/kg, or 1×10⁶ cells/kg, 2×10⁶ cells/kg, 5×10⁶ cells/kg, or 10×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁷ T cells/kg body weight.

An anti-MUC16 antibody agent or antigen-binding fragment thereof can be administered on multiple occasions. Intervals between single dosages can be 1 week, 2 weeks, 3 weeks, 4 weeks, 1 month, 2 months, 3 months, 6 months, 1 year, or 2 years.

### Combination Therapies

In some embodiments, the methods provided herein for treating cancer (e.g., ovarian cancer, pancreatic cancer, lung cancer, breast cancer, fallopian tube cancer, uterine (e.g., endometrial) cancer, or primary peritoneum cancer) in a subject, comprising administering to a subject in need thereof a pharmaceutical composition comprising an anti-MUC16 antibody agent or an antigen-binding fragment thereof described herein, further comprise administering to the subject one or more additional therapeutic agents. In some embodiments, the additional therapeutic agent is for treating the cancer in the subject (e.g., ovarian cancer, pancreatic cancer, lung cancer, breast cancer, fallopian tube cancer, uterine (e.g., endometrial) cancer, and primary peritoneum cancer). In some embodiments, the additional therapeutic agent is for treating any side effects of treatment with an anti-MUC16 antibody agent or an antigen-binding fragment thereof described herein.

In some embodiments, the additional agent is an agent used to treat ovarian cancer. In some embodiments, the additional agent is an agent used to treat pancreatic cancer. In some embodiments, the additional agent is an agent used to treat lung cancer. In some embodiments, the additional agent is an agent used to treat breast cancer. In some embodiments, the additional agent is an agent used to treat fallopian tube cancer. In some embodiments, the additional agent is an agent used to treat uterine (e.g., endometrial) cancer. In some embodiments, the additional agent is an agent used to treat primary peritoneum cancer.

An anti-MUC16 antibody agent or antigen-binding fragment thereof described herein can be administered with an additional therapeutic agent concurrently or sequentially (before and/or after). The antibody or antigen binding fragment thereof and the additional therapeutic agent can be administered in the same or different compositions, and by the same or different routes of administration. A first therapy (which is an anti-MUC16 antibody agent or an antigen-binding fragment thereof described herein, or the additional therapeutic agent) can be administered prior to (e.g., 5 minutes, 15 minutes, 30 minutes, 45 minutes, 1 hour, 2 hours, 4 hours, 6 hours, 12 hours. 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes. 1 hour, 2 hours, 4 hours, 6 hours, 12 hours, 24 hours. 48 hours, 72 hours, 96 hours, 1 week, 2 weeks, 3 weeks, 4 weeks, 5 weeks, 6 weeks, 8 weeks, or 12 weeks after) the administration of the second therapy (the anti-MUC16 antibody agent or an antigen-binding fragment thereof described herein, or the additional therapeutic agent) to a subject with cancer (e.g., ovarian cancer, pancreatic cancer, lung cancer, breast cancer, fallopian tube cancer, uterine (e.g., endometrial) cancer, and primary peritoneum cancer). In certain embodiments, an additional therapeutic agent administered to a subject in combination with an anti-MUC16 antibody agent or an antigen-binding fragment thereof described herein is administered in the same composition (pharmaceutical composition). In other embodiments, an additional therapeutic agent administered in combination with an anti-MUC16 antibody agent or an antigen-binding fragment thereof described herein is administered to a subject in a different composition than the anti-MUC16 antibody agent or an antigen-binding fragment thereof described herein (e.g., two or more pharmaceutical compositions are used).

### Exemplary Patient Populations

A subject treated in accordance with the methods provided herein can be any mammal, such as a rodent, a cat, a canine, a horse, a cow, a pig, a monkey, a primate, or a human, etc. In some embodiments, the subject is a human. In some embodiments, the subject is a canine. As used herein, the terms “subject” and “patient” are used interchangeably.

In certain embodiments, a subject treated in accordance with the methods provided herein has been diagnosed with a MUC16-positive cancer, including but not limited to, ovary, lung, pancreas, breast, uterine, fallopian tube, or primary peritoneum cancer, of cancer of any other tissue that expresses the MUC16.

### Articles of Manufacture and Kits

In some embodiments of the invention, there is provided an article of manufacture containing materials useful for the treatment of a cancer characterized by high MUC16 expression and/or high aerobic glycolysis (e.g., kidney cancer, cervical cancer, or prostate cancer), or for delivering an anti-MUC16 antibody agent (such as a full-length anti-MUC16 antibody) to a cell expressing MUC16 on its surface. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-MUC16 antibody agent of the invention. The label or package insert indicates that the composition is used for treating the particular condition. The label or package insert will further comprise instructions for administering the anti-MUC16 antibody agent composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating cancer (such as HCC, melanoma, lung squamous cell carcinoma, ovarian carcinoma, yolk sac tumor, choriocarcinoma, neuroblastoma, hepatoblastoma, Wilms' tumor, testicular nonseminomatous germ cell tumor, gastric carcinoma, or liposarcoma).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a cancer characterized by high MUC16 expression and/or high aerobic glycolysis (e.g., kidney cancer, cervical cancer, or prostate cancer), or for delivering an anti-MUC16 antibody agent (such as a full-length anti-MUC16 antibody) to a cell expressing MUC16 on its surface, optionally in combination with the articles of manufacture. Kits of the invention include one or more containers comprising an anti-MUC16 antibody agent composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-MUC16 antibody agent (such as a full-length anti-MUC16 antibody). In some embodiments, the kit comprises a) a composition comprising an anti-MUC16 antibody agent, and b) an effective amount of at least one other agent, wherein the other agent enhances the effect (e.g., treatment effect, detecting effect) of the anti-MUC16 antibody agent. In some embodiments, the kit comprises a) a composition comprising an anti-MUC16 antibody agent, and b) instructions for administering the anti-MUC16 antibody agent composition to an individual for treatment of a cancer characterized by high MUC16 expression and/or high aerobic glycolysis (e.g., kidney cancer, cervical cancer, or prostate cancer). In some embodiments, the kit comprises a) a composition comprising an anti-MUC16 antibody agent, b) an effective amount of at least one other agent, wherein the other agent enhances the effect (e.g., treatment effect, detecting effect) of the anti-MUC16 antibody agent, and c) instructions for administering the anti-MUC16 antibody agent composition and the other agent(s) to an individual for treatment of a cancer characterized by high MUC16 expression and/or high aerobic glycolysis (e.g., kidney cancer, cervical cancer, or prostate cancer). The anti-MUC16 antibody agent and the other agent(s) can be present in separate containers or in a single container. For example, the kit may comprise one distinct composition or two or more compositions wherein one composition comprises an anti-MUC16 antibody agent and another composition comprises another agent.

In some embodiments, the kit comprises a nucleic acid (or set of nucleic acids) encoding an anti-MUC16 antibody agent (such as a full-length anti-MUC16 antibody). In some embodiments, the kit comprises a) a nucleic acid (or set of nucleic acids) encoding an anti-MUC16 antibody agent, and b) a host cell for expressing the nucleic acid (or set of nucleic acids). In some embodiments, the kit comprises a) a nucleic acid (or set of nucleic acids) encoding an anti-MUC16 antibody agent, and b) instructions for i) expressing the anti-MUC16 antibody agent in a host cell, ii) preparing a composition comprising the anti-MUC16 antibody agent, and iii) administering the composition comprising the anti-MUC16 antibody agent to an individual for the treatment of a cancer characterized by high MUC16 expression and/or high aerobic glycolysis (e.g., kidney cancer, cervical cancer, or prostate cancer). In some embodiments, the kit comprises a) a nucleic acid (or set of nucleic acids) encoding an anti-MUC16 antibody agent, b) a host cell for expressing the nucleic acid (or set of nucleic acids), and c) instructions for i) expressing the anti-MUC16 antibody agent in the host cell, ii) preparing a composition comprising the anti-MUC16 antibody agent, and iii) administering the composition comprising the anti-MUC16 antibody agent to an individual for the treatment of a cancer characterized by high MUC16 expression and/or high aerobic glycolysis (e.g., kidney cancer, cervical cancer, or prostate cancer).

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-MUC16 antibody agent compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-MUC16 antibody agent (such as a full-length anti-MUC16 antibody) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks. 3 months, 4 months, S months, 7 months, 8 months, 9 months, or more. Kits may also include multiple unit doses of the anti-MUC16 antibody agent and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

The present technology is further illustrated by the following Examples, which should not be construed as limiting in any way. The following Examples demonstrate the preparation, characterization, and use of illustrative anti-MUC16 antibodies of the present technology. The following Examples demonstrate the production of human and bispecific antibodies of the present technology, and characterization of their binding specificities and in vivo biological activities.

### Example 1: Selection and Characterization of scFvs Specific for Human MUC16

This example demonstrates the selection and characterization of human scFvs specific for human MUC16 (hMUC16) from a collection of human scFv antibody phage display libraries. In particular, this example demonstrates the selection of human scFvs that specifically bind to ectodomain of hMUC16 (MUC16-C114) in its native format (i.e., cell surface-bound MUC16). The example also demonstrates the further selection of human scFvs that target the crucial N-glycosylation site (N30 in c114 or N1806 in a full-length mature MUC16 protein) on the MUC16 ectodomain to inhibit the glycosylation dependent effects of MUC16 on metastasis and invasion. A structural representation of native MUC16 and the truncated MUC16-C114, along with its amino acid sequence, is shown in FIG. 1. The scFvs were selected based on high selectivity for human MUC16 via panning against cell surface-bound MUC16-C114. These human anti-MUC16 scFvs provide a valuable source of antibody components for the construction of anti-MUC16 antibody agents in various formats, e.g., full-length IgG, bi-specific anti-MUC16 antibodies, multi-specific anti-MUC16 antibodies, and the like.

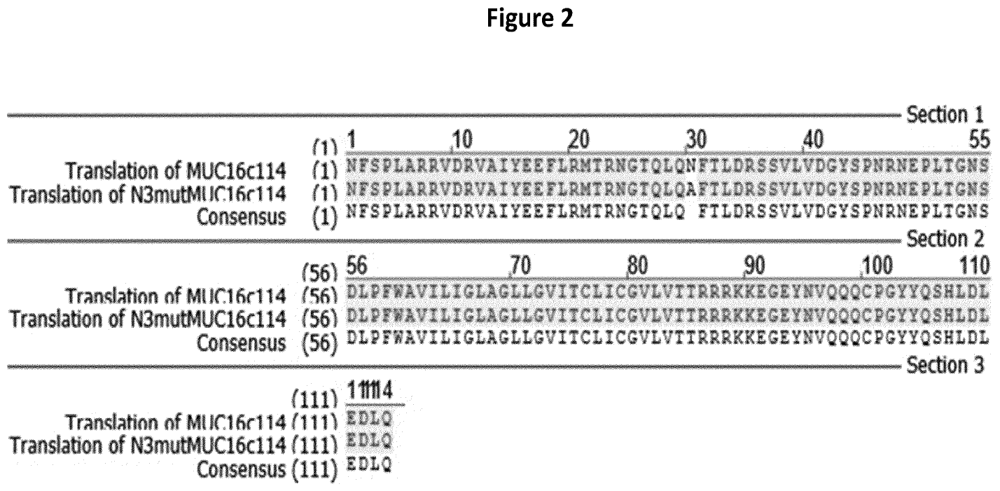

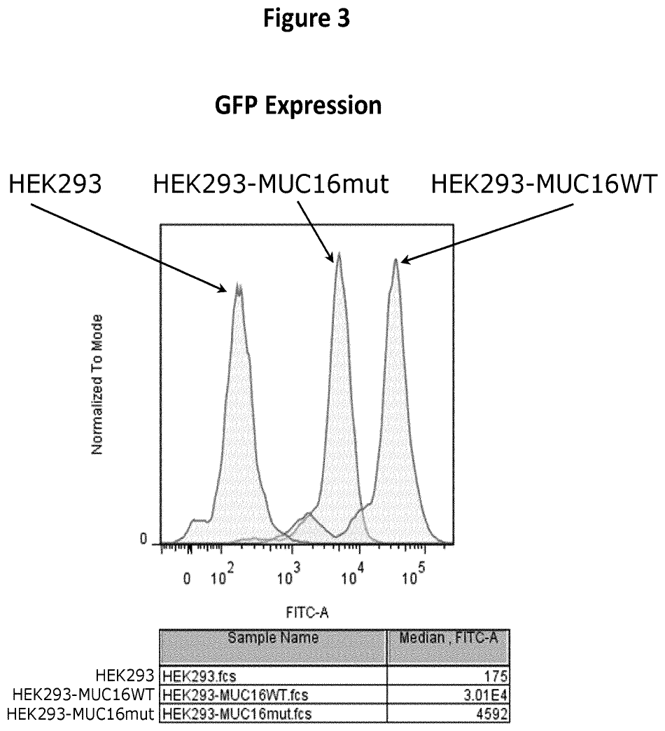

Two HEK293 stable cell lines expressing MUC16 proteins were generated for membrane bound expression of MUC16 for use in the selection and characterization of scFvs that specifically target N30 N-glycosylation on the MUC16 ectodomain. One cell line was generated to express a wildtype MUC16-C114-GFP fusion protein (HEK293-MUC16WT), and one cell line was generated to express an N30 mutant MUC16-C114-GFP fusion protein (HEK293-MUC16mut). The alignment between wildtype MUC16-C114 and the N30 mutant MUC16-C114 ectodomains is shown in FIG. 2. As shown in FIG. 2, the N30 mutant MUC16-C114 has an N30A substitution. MUC16 expression of the HEK293-MUC16WT and HEK293-MUC16mut cells was confirmed by measuring GFP expression by fluorescence activated cell sorting (FACS). The parental HEK293 cell line showed no GFP signal. By contrast, the HEK293-MUC16WT and the HEK293-MUC16mut cell lines exhibited GFP expression, though HEK293-MUC16WT had a stronger GFP signal than the HEK293-MUC16mut cell line (FIG. 3). The HEK293-MUC16WT cells showed a GFP signal of 170× mean fluorescence intensity (MFI) increase, while the MUC16mut cells showed a 26× MFI increase.

A collection of human scFv antibody phage display libraries (diversity over 10×10¹⁰) constructed by Eureka Therapeutics (trademarked as E-ALPHA® phage libraries) was used for the selection of human scFvs specific for hMUC16.

The E-ALPHA® scFv phage libraries were screened (panned) against hMUC16 by co-incubation with negative control parental HEK293 cells and MUC16-C114-GFP fusion protein expressing HEK293 cells (HEK293-MUC16WT). After extended washing with PBS, HEK293-MUC16WT cells with bound scFv antibody phage were spun down. The bound clones were then eluted and used for 2-3 additional rounds of panning to enrich for scFv phage clones that bound MUC16 specifically. The bound clones were then eluted and used to infect E. coli XLI-Blue cells. The phage clones expressed in bacteria were then purified.

540 phage clones identified from the cell panning were then tested by FACS analysis for binding to HEK293-MUC16WT cells. Briefly, 0.2 million cells (in PBS+5% FBS+0.05% NaN₃) were incubated for 2 h at 4° C. with 50 μl of ˜1.0×10″ pfu/mL phage in PBS. FACS was carried out using primary antibody mouse anti-M13 mAb (Thermo #MAI-12900) and secondary antibody PE anti-mouse IgG (Vectors Lab #EI-2007). 53 unique clones were identified and 40 clones demonstrated specific binding for HEK293-MUC16 WT cells.

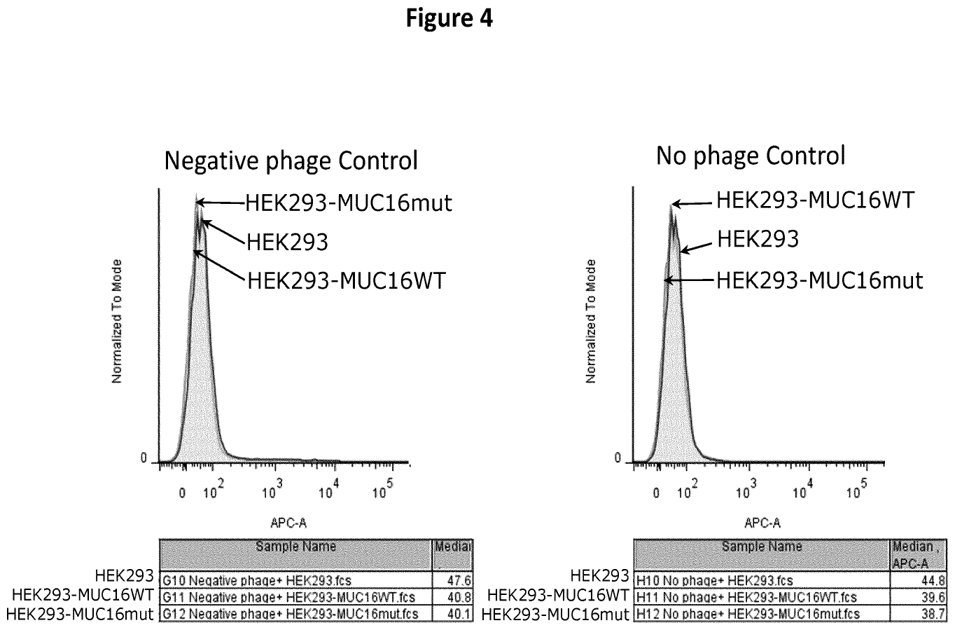

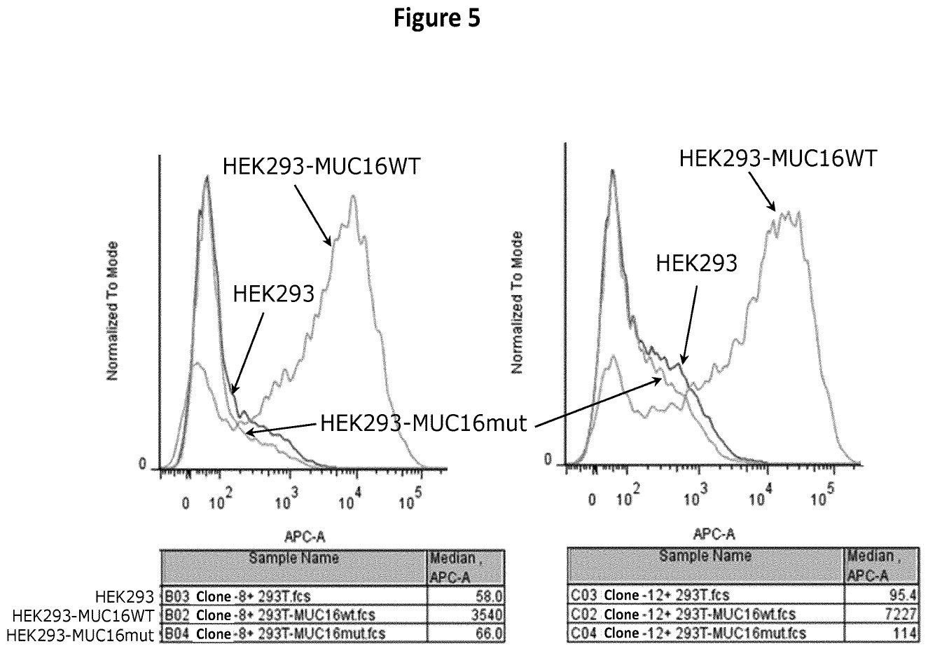

The 40 MUC16 specific clones were tested for their binding to HEK293-MUC16WT. HEK293-MUC16mut, and parental HEK293 cells. FIG. 4 shows the results of FACS analysis with a negative phage control and a no phage control for all three cell lines. FACS analysis with each of the 40 clones showed that all of the 40 clones showed binding towards HEK293-MUC16WT while 16 of the 40 clones showed minimum binding towards HEK293-MUC16mut. These clones were thus specific for the N-glycosylation site (N30). FIG. 5 shows the results for two exemplary clones, clone 8 and clone 12.

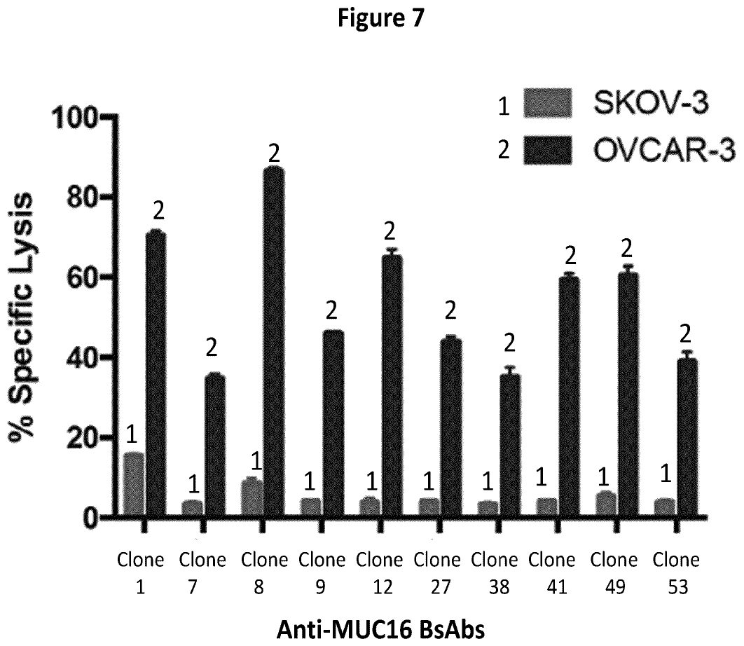

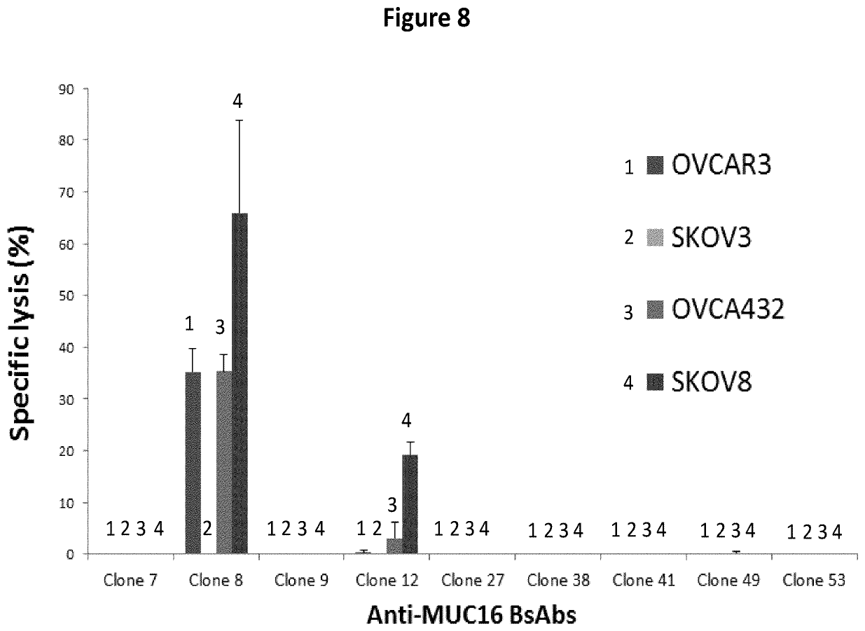

The top 9 clones among the 16 were then tested for binding to MUC16⁺ cancer cell lines OVCAR3, SKOV8, and OVCA432. Anti-MUC16 clones 8 and 12 bound most specifically to MUC16+ cancer cell lines but not to a MUC16⁻ cancer cell line SKOV3 (FIG. 8). Several other clones bound specifically to MUC16⁺ cancer cell lines as well, although at lower specificities (FIG. 7).

### Example 2: Generation of Anti-MUC16 Bispecific Antibodies

The example describes the generation of anti-MUC16 bi-specific antibodies (BsAbs) from the anti-MUC16 scFvs identified in Example 1. In this example, a single-chain BsAb comprising anti-MUC16 scFv at the N-terminal end and an anti-human CD3c scFv of a mouse monoclonal antibody at the C-terminal end was generated. An anti-MUC16 clone 8 BsAb and anti-MUC16 clone 12 BsAb were generated by cloning DNA fragments encoding the anti-MUC16 scFv and the anti-human CD38 scFv antibody derived from parental clone L2K into an expression vector using standard DNA technology. A hexahistidine (His) tag (SEQ ID NO: 33) was inserted downstream of the anti-MUC16 BsAb at the C-terminal end for antibody purification and detection.

Chinese hamster ovary (CHO) cells were transfected with the anti-MUC16 BsAb expression vector and stable expression was achieved by standard drug selection with methionine sulfoximine (MSX), a glutamine synthetase (GS)-based method (Fan, et al., Biotechnology Bioengineering. 109 (4), 1007-1005 (2012)). CHO cell supernatants containing secreted anti-MUC16 BsAb molecules were collected. Anti-MUC16 BsAb was purified using HISTRAP HP column (GE healthcare) by FPLC AKTA system. Briefly, CHO cell culture was clarified and loaded onto the column with low imidazole concentration (20 mM), and then an isocratic high imidazole concentration elution buffer (500 mM) was used to elute the bound anti-MUC16 bi-specific antibody protein. The major bands for the clone 8 BsAb and clone 12 BsAb were observed around 50 kDa by SDS-PAGE, indicating that the BsAbs were successfully purified.

### Example 3: Anti-MUC16 BsAb—MUC16+ Cell Specificity

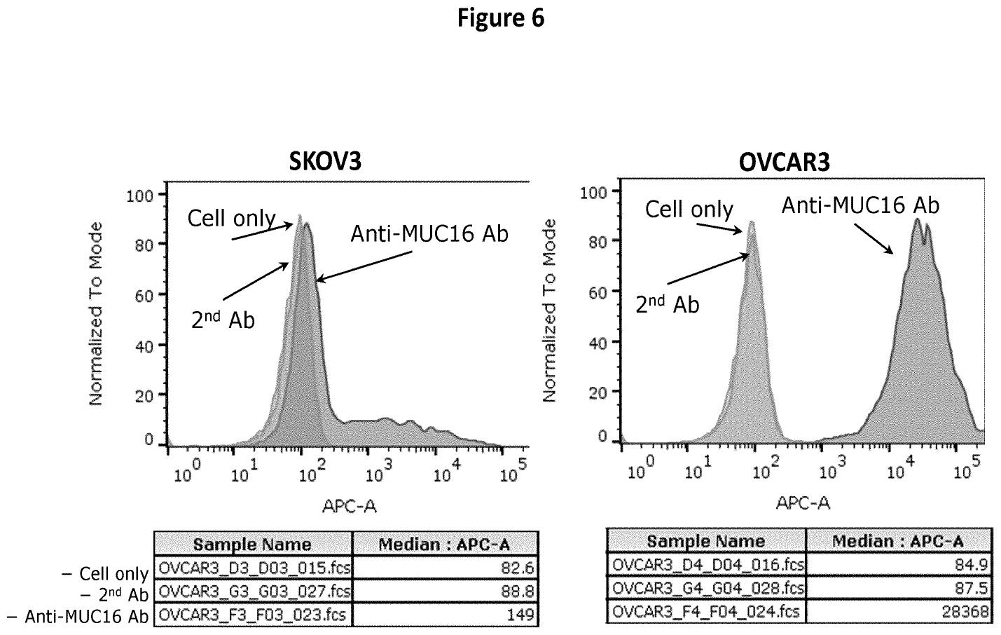

In this example, the specificity of the anti-MUC16 BsAbs for binding to cancer cells that express MUC16 was assessed. In one study, two target cell lines were employed, a MUC16⁺ OVCAR3 cell line and a MUC16 SKOV3 cell line. OVCAR3 and SKOV3 cell lines were obtained through the American Type Culture Collection (ATCC®, Manassas, VA) and sustained in culture according to the ATCC® literature. FACS analysis of anti-MUC16 antibody binding to the two target cell lines was performed to confirm that antibody binding was observed only with the MUC16+ OVCAR3 cell line. SKOV3 or OVCAR3 cell lines were incubated with the anti-MUC16 Ab followed by a secondary antibody or with a secondary antibody alone as a control. Data for the anti-MUC16 clone & BsAb is shown in FIG. 6. The MUC16+ OVCAR3 cell line showed about a 300× MFI increase in binding over the control cells while SKOV3 exhibited only a minimum signal.

### Example 4: Anti-MUC16 BsAb-Directed Cell Cytotoxicity

In this example, the ability of the anti-MUC16 BsAbs to induce MUC16-specific cell toxicity was assessed. Anti-MUC16 clone 8 BsAb and anti-MUC16 clone 12 BsAb were incubated at a concentration of 0.2 μg/ml with either the MUC16⁺ OVCAR3 target cell line or the MUC16 SKOV3 target cell line and human activated T cells at an effector: target (E:T) ratio of 5:1 for 16 hours. The cytotoxicity was measured by lactate dehydrogenase (LDH) release assay. As shown in FIG. 7, the clone 8 and clone 12 BsAbs were able to induce cell lysis of OVCAR3 cells at a level of about 90% and 65%, respectively, while cell lysis of SKOV3 was minimal, indicating that the MUCH6′ target specificity is required for the T cell activation. Thus, both clone 8 BsAb and clone 12 BsAb induced potent and specific killing of a MUC16′ cancer cell line.

In a separate study, four target cell lines were employed, a MUCH6′ OVCAR3 cell line, a MUC16⁻ SKOV3 cell line, a MUC16⁺ SKOV8 cell line, and a MUC16⁺ OVCA432 cell line. Anti-MUC16 clone & BsAb and anti-MUC16 clone 12 BsAb were incubated at a concentration of 0.2 ng/ml with a target cell line and human activated T cells at an effector: target (E:T) ratio of 3:1 for 16 hours. The cytotoxicity was measured by LDH release assay. Lower percentages of cell lysis for the MUC16+ cell lines were observed at the lower E:T ratio of 3:1 (FIG. 8), compared to the E:T ratio of 5:1. Cell lysis of MUC16 SKOV3 was minimal in this study as well, further support that the MUC16+ target specificity of the BsAb is required for the T cell activation.

### Example 5: Therapy of Human MUC16⁺ Metastatic Ovarian Cancer in NSG Mice

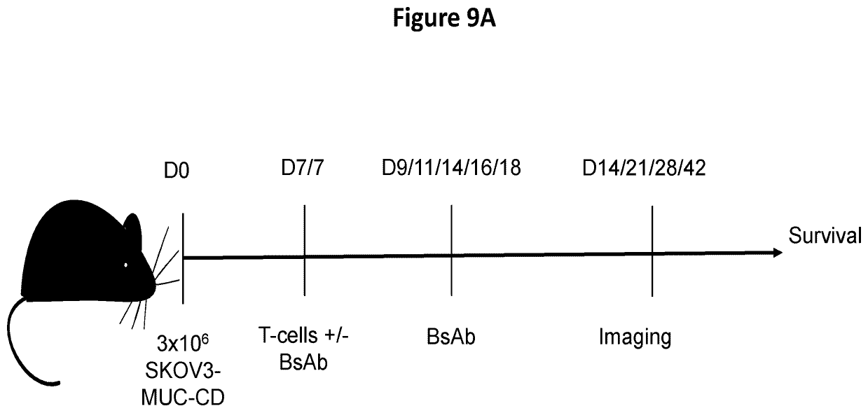

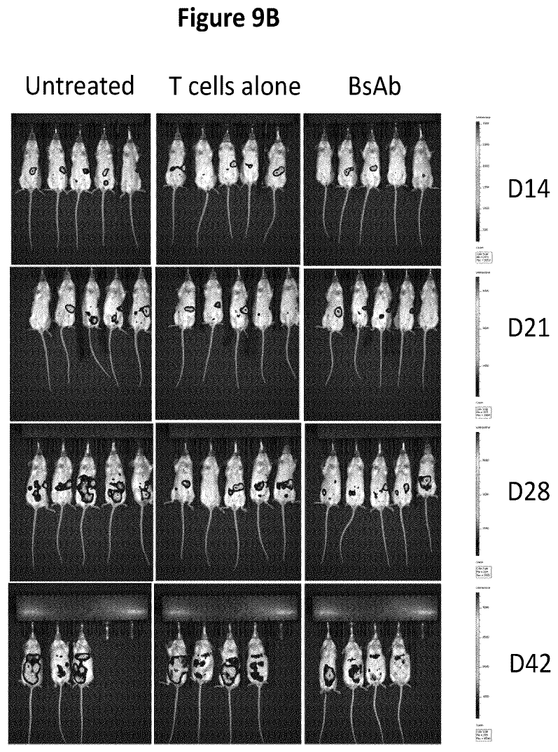

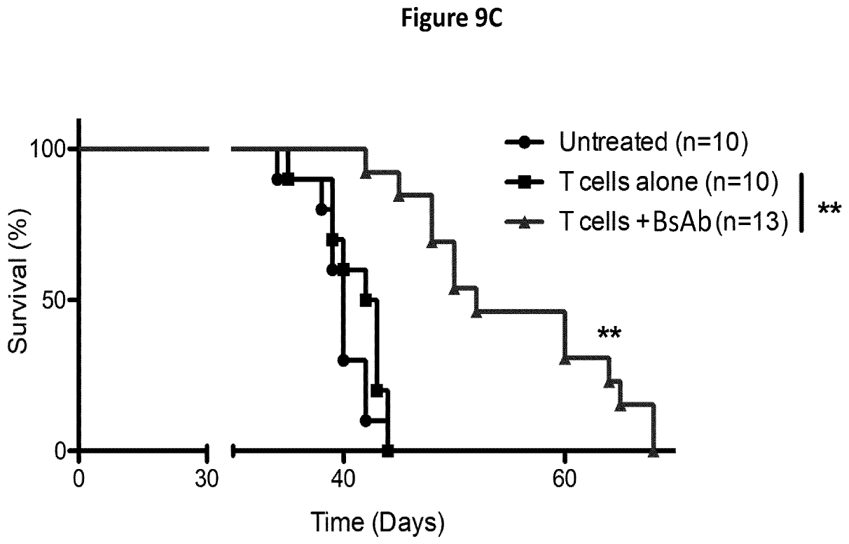

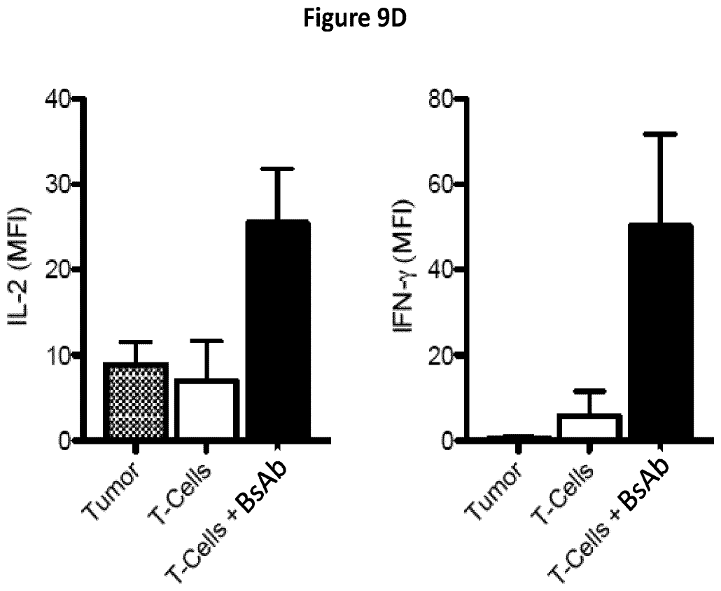

In this example, the in vivo therapeutic efficacy of anti-MUC16 BsAb in a mouse xenograft model of metastatic ovarian cancer was assessed. Female NSG™ mice between 6-8 weeks old were injected on day 0 (DO) intraperitoneally (i.p.) with 3×10⁶ SKOV3-MUC-CD tumor cells that are modified to express MUC16-C114 and GFP-LUC. These mice were then treated intravenously (i.v.) with 1×10⁷ human T cells on day 7 (D7) and i.p with 5 μg of anti-MUC16 clone 8 BsAb. Additional treatments with 5 μg of the BsAb were administered i.p. on D9, D11, D14, D16, and D18 for a total of six BsAb treatments. Animals were imaged on D14, D21, D28, and D42. The experimental schema of the SKOV3-MUC-CD and BsAb injections is shown in FIG. 9A.

Animals treated with the anti-MUC16 clone & BsAb exhibited delayed disease progression compared to untreated mice or mice treated with T-cells alone (FIG. 9B). FIG. 9C shows the survival curves of the tumor-bearing mice. Treatment with the anti-MUC16 clone 8 BsAb significantly prolonged survival in tumor-bearing mice compared to T-cell therapy or no treatment. Tumor-bearing mice treated with T-cells and anti-MUC16 BsAbs also showed significantly elevated levels of systemic IL-2 and IFN-γ 7 days after treatment indicating an induction of an anti-tumor immune response (FIG. 9D). These results demonstrate that administration of anti-MUC16 BsAb delays disease progression and improves survival in a xenogeneic model of MUC16+ metastatic ovarian cancer.

### Example 6: Generation of Full-Length Human IgG Anti-MUC16 Antibodies

Full-length human IgG1 of the selected phage clones are produced, for example, in HEK293 and CHO cell lines as described (Tomimatsu, K. et al., Biosci. Biotechnol. Biochem. 73 (7): 1465-1469, 2009). In brief, antibody variable regions from the phage clones are subcloned into mammalian expression vectors, with matching human lambda light chain constant region (SEQ ID NO: 31) and human IgG1 constant region (SEQ ID NO: 28) sequences (see Table 5). Molecular weight of the purified full-length IgG1 antibodies can be measured under both reducing and non-reducing conditions by electrophoresis. SDS-PAGE of purified IgG1 antibodies can be performed to determine protein purity.

### Table 5

TABLE 5

| Phage clone | HC Variable | HC Constant | LC Variable | LC Constant |
|---|---|---|---|---|
| 8 | SEQ ID NO: 2 | SEQ ID NO: 28 | SEQ ID NO: 3 | SEQ ID NO: 31 |
| 12 | SEQ ID NO: 10 | SEQ ID NO: 28 | SEQ ID NO: 11 | SEQ ID NO: 31 |

### Example 7: Characterization of Full-Length Human IgG Anti-MUC16 Antibodies

Anti-MUC16 IgG antibodies are tested for binding to MUC16-expressing cells, such as HEK293-MUC16 wt cells or MUC16′ cell lines such as OVCAR3, SKOV8 cell line, or OVCA432 cell line, by flow cytometry. Dose dependence of binding is tested. Briefly, MUC16-expressing cells are incubated with varying amounts of the anti-human MUC16 IgG antibodies, for example, at 10, 3.3, 1.1, 0.37, 0.12, 0.041, 0.014 or 0 μg/ml, on ice for 1 hour. The anti-MUC16 IgG antibodies are evaluated for their affinity towards the MUC16-expressing cells by EC50 of the dose dependence curve (MFI vs. antibody concentration). Furthermore, apparent Ko is determined based on EC50 value. Binding affinity of the anti-MUC16 IgG antibodies can be determined, for example, by FORTEBIO®.

### Example 8: Characterization of Full-Length Human IgG Anti-MUC16 Antibodies

The ability of anti-MUC16 clone & and anti-MUC16 clone 12 to inhibit Matrigel invasion is evaluated. The anti-MUC16 monoclonal antibody 4H11 is used as a negative control and Matrigel invasion assays are performed with SKOV3 ovarian cancer stable cell lines expressing phrGFP or phr-GFP-MUC16-C114 by incubating the cells in the presence or absence of 4H11, clone 8, or clone 12. Clone 8 and Clone 12 inhibit MUC16-C114-induced Matrigel invasion. In contrast, the monoclonal anti-MUC16 antibody 4H11 does not inhibit MUC16-C114-induced Matrigel invasion. These data demonstrate that, in contrast to the monoclonal antibody 4H11, clone 8 and clone 12 block MATRIGEL® invasion.

### Table 6: Table of Sequences

TABLE 6
Table of Sequences

| SEQ ID NO | Description | Sequence |
|---|---|---|
| 1 | hMUC16 | MLKPSGLPGSSSPTRSLMTGSRSTKATPEMDSGLTGATLSPKTSTGA |
| | (Immature) | IVVTEHTLPFTSPDKTLASPTSSVVGRTTQSLQVMSSALPESTSRGM |
| | | THSEQRTSPSLSPQVNGTPSRNYPATSMVSGLSSPRTRTSSTEGNFT |
| | | KEASTYTLTVETTSGPVTEKYTVPTETSTTEGDSTETPWDTRYIPVK |
| | | ITSPMKTFADSTASKENAPVSMTPAETTVTDSHTPGRTNPSFGTLYS |
| | | SFLDLSPKGTPNSRGETSLELILSTTGYPFSSPEPGSAGHSRISTSAPL |
| | | SSSASVLDNKISETSIFSGQSLTSPLSPGVPEARASTMPNSAIPFSMTL |
| | | SNAETSAERVRSTISSLGTPSISTKQTAETILTFHAFAETMDIPSTHIA |
| | | KTLASEWLGSPGTLGGTSTSALTTTSPSTTLVSEETNTHHSTSGKET |
| | | EGTLNTSMTPLETSAPGEESEMTATLVPTLGFTTLDSKIRSPSQVSSS |
| | | HPTRELRTTGSTSGRQSSSTAAHGSSDILRATTSSTSKASSWTSESTA |
| | | QQFSEPQHTQWVETSPSMKTERPPASTSVAAPITTSVPSVVSGFTTL |
| | | KTSSTKGIWLEETSADTLIGESTAGPTTHQFAVPTGISMTGGSSTRG |
| | | SQGTTHLLTRATASSETSADLTLATNGVPVSVSPAVSKTAAGSSPPG |
| | | GTKPSYTMVSSVIPETSSLQSSAFREGTSLGLTPLNTRHPFSSPEPDS |
| | | AGHTKISTSIPLLSSASVLEDKVSATSTFSHHKATSSITTGTPEISTKT |
| | | KPSSAVLSSMTLSNAATSPERVRNATSPLTHPSPSGEETAGSVLTLS |
| | | TSAETTDSPNIHPTGTLTSESSESPSTLSLPSVSGVKTTFSSSTPSTHLF |
| | | TSGEETEETSNPSVSQPETSVSRVRTTLASTSVPTPVFPTMDTWPTR |
| | | SAQFSSSHLVSELRATSSTSVTNSTGSALPKISHLTGTATMSQTNRD |
| | | TFNDSAAPQSTTWPETSPRFKTGLPSATTTVSTSATSLSATVMVSKF |
| | | TSPATSSMEATSIREPSTTILTTETTNGPGSMAVASTNIPIGKGYITEG |
| | | RLDTSHLPIGTTASSETSMDFTMAKESVSMSVSPSQSMDAAGSSTP |
| | | GRTSQFVDTFSDDVYHLTSREITIPRDGTSSALTPQMTATHPPSPDP |
| | | GSARSTWLGILSSSPSSPTPKVTMSSTFSTQRVTTSMIMDTVETSRW |
| | | NMPNLPSTTSLTPSNIPTSGAIGKSTLVPLDTPSPATSLEASEGGLPTL |
| | | STYPESTNTPSIHLGAHASSESPSTIKLTMASVVKPGSYTPLTFPSIET |
| | | HIHVSTARMAYSSGSSPEMTAPGETNTGSTWDPTTYITTTDPKDTSS |
| | | AQVSTPHSVRTLRTTENHPKTESATPAAYSGSPKISSSPNLTSPATK |
| | | AWTITDTTEHSTQLHYTKLAEKSSGFETQSAPGPVSVVIPTSPTIGSS |
| | | TLELTSDVPGEPLVLAPSEQTTITLPMATWLSTSLTEEMASTDLDISS |
| | | PSSPMSTFAIFPPMSTPSHELSKSEADTSAIRNTDSTTLDQHLGIRSLG |
| | | RTGDLTTVPITPLTTTWTSVIEHSTQAQDTLSATMSPTHVTQSLKDQ |
| | | TSIPASASPSHLTEVYPELGTQGRSSSEATTFWKPSTDTLSREIETGP |
| | | TNIQSTPPMDNTTTGSSSSGVTLGIAHLPIGTSSPAETSTNMALERRS |
| | | STATVSMAGTMGLLVTSAPGRSISQSLGRVSSVLSESTTEGVTDSSK |
| | | GSSPRLNTQGNTALSSSLEPSYAEGSQMSTSIPLTSSPTTPDVEFIGGS |
| | | TFWTKEVTTVMTSDISKSSARTESSSATLMSTALGSTENTGKEKLR |
| | | TASMDLPSPTPSMEVTPWISLTLSNAPNTTDSLDLSHGVHTSSAGTL |
| | | ATDRSLNTGVTRASRLENGSDTSSKSLSMGNSTHTSMTYTEKSEVS |
| | | SSIHPRPETSAPGAETTLTSTPGNRAISLTLPFSSIPVEEVISTGITSGPD |
| | | INSAPMTHSPITPPTIVWTSTGTIEQSTQPLHAVSSEKVSVQTQSTPY |
| | | VNSVAVSASPTHENSVSSGSSTSSPYSSASLESLDSTISRRNAITSWL |
| | | WDLTTSLPTTTWPSTSLSEALSSGHSGVSNPSSTTTEFPLFSAASTSA |
| | | AKQRNPETETHGPQNTAASTLNTDASSVTGLSETPVGASISSEVPLP |
| | | MAITSRSDVSGLTSESTANPSLGTASSAGTKLTRTISLPTSESLVSFR |
| | | MNKDPWTVSIPLGSHPTTNTETSIPVNSAGPPGLSTVASDVIDTPSD |
| | | GAESIPTVSFSPSPDTEVTTISHFPEKTTHSFRTISSLTHELTSRVTPIP |
| | | GDWMSSAMSTKPTGASPSITLGERRTITSAAPTTSPIVLTASFTETST |
| | | VSLDNETTVKTSDILDARKTNELPSDSSSSSDLINTSIASSTMDVTKT |
| | | ASISPTSISGMTASSSPSLFSSDRPQVPTSTTETNTATSPSVSSNTYSL |
| | | DGGSNVGGTPSTLPPFTITHPVETSSALLAWSRPVRTFSTMVSTDTA |
| | | SGENPTSSNSVVTSVPAPGTWTSVGSTTDLPAMGFLKTSPAGEAHS |
| | | LLASTIEPATAFTPHLSAAVVTGSSATSEASLLTTSESKAIHSSPQTPT |
| | | TPTSGANWETSATPESLLVVTETSDTTLTSKILVTDTILFSTVSTPPS |
| | | KFPSTGTLSGASFPTLLPDTPAIPLTATEPTSSLATSFDSTPLVTIASDS |
| | | LGTVPETTLTMSETSNGDALVLKTVSNPDRSIPGITIQGVTESPLHPS |
| | | STSPSKIVAPRNTTYEGSITVALSTLPAGTTGSLVFSQSSENSETTAL |
| | | VDSSAGLERASVMPLTTGSQGMASSGGIRSGSTHSTGTKTFSSLPLT |
| | | MNPGEVTAMSEITTNRLTATQSTAPKGIPVKPTSAESGLLTPVSASS |
| | | SPSKAFASLTTAPPTWGIPQSTLTFEFSEVPSLDTKSASLPTPGQSLN |
| | | TIPDSDASTASSSLSKSPEKNPRARMMTSTKAISASSFQSTGFTETPE |
| | | QSASPSMACHEPRVPTSGTGDPRYASESMSYPDPSKASSAMTSTSL |
| | | ASKLTTLFSTGQAARSGSSSSPISLSTEKETSFLSPTASTSRKTSLFLG |
| | | PSMARQPNILVHLQTSALTLSPTSTLNMSQEEPPELTSSQTIAEEEGT |
| | | TAETQTLTFTPSETPTSLLPVSSPTEPTARRKSSPETWASSISVPAKTS |
| | | LVETTDGTLVTTIKMSSQAAQGNSTWPAPAEETGSSPAGTSPGSPE |
| | | MSTTLKIMSSKEPSISPEIRSTVRNSPWKTPETTVPMETTVEPVTLQS |
| | | TALGSGSTSISHLPTGTTSPTKSPTENMLATERVSLSPSPPEAWTNLY |
| | | SGTPGGTRQSLATMSSVSLESPTARSITGTGQQSSPELVSKTTGMEF |
| | | SMWHGSTGGTTGDTHVSLSTSSNILEDPVTSPNSVSSLTDKSKHKT |
| | | ETWVSTTAIPSTVLNNKIMAAEQQTSRSVDEAYSSTSSWSDQTSGS |
| | | DITLGASPDVTNTLYITSTAQTTSLVSLPSGDQGITSLTNPSGGKTSS |
| | | ASSVTSPSIGLETLRANVSAVKSDIAPTAGHLSQTSSPAEVSILDVTT |
| | | APTPGISTTITTMGTNSISTTTPNPEVGMSTMDSTPATERRTTSTEHP |
| | | STWSSTAASDSWTVTDMTSNLKVARSPGTISTMHTTSFLASSTELD |
| | | SMSTPHGRITVIGTSLVTPSSDASAVKTETSTSERTLSPSDTTASTPIS |
| | | TFSRVQRMSISVPDILSTSWTPSSTEAEDVPVSMVSTDHASTKTDPN |
| | | TPLSTFLFDSLSTLDWDTGRSLSSATATTSAPQGATTPQELTLETMIS |
| | | PATSQLPFSIGHITSAVTPAAMARSSGVTFSRPDPTSKKAEQTSTQLP |
| | | TTTSAHPGQVPRSAATTLDVIPHTAKTPDATEQRQGQTALTTEARA |
| | | TSDSWNEKEKSTPSAPWITEMMNSVSEDTIKEVTSSSSVLRTLNTLD |
| | | INLESGTTSSPSWKSSPYERIAPSESTTDKEAIHPSTNTVETTGWVTS |
| | | SEHASHSTIPAHSASSKLTSPVVTTSTREQAIVSMSTTTWPESTRART |
| | | EPNSFLTIELRDVSPYMDTSSTTQTSIISSPGSTAITKGPRTEITSSKRIS |
| | | SSFLAQSMRSSDSPSEAITRLSNFPAMTESGGMILAMQTSPPGATSL |
| | | SAPTLDTSATASWTGTPLATTQRFTYSEKTTLFSKGPEDTSQPSPPS |
| | | VEETSSSSSLVPIHATTSPSNILLTSQGHSPSSTPPVTSVFLSETSGLG |
| | | KTTDMSRISLEPGTSLPPNLSSTAGEALSTYEASRDTKAIHHSADTA |
| | | VTNMEATSSEYSPIPGHTKPSKATSPLVTSHIMGDITSSTSVFGSSET |
| | | TEIETVSSVNQGLQERSTSQVASSATETSTVITHVSSGDATTHVTKT |
| | | QATFSSGTSISSPHQFITSTNTFTDVSTNPSTSLIMTESSGVTITTQTGP |
| | | TGAATQGPYLLDTSTMPYLTETPLAVTPDFMQSEKTTLISKGPKDV |
| | | SWTSPPSVAETSYPSSLTPFLVTTIPPATSTLQGQHTSSPVSATSVLTS |
| | | GLVKTTDMLNTSMEPVTNSPQNLNNPSNEILATLAATTDIETIHPSI |
| | | NKAVTNMGTASSAHVLHSTLPVSSEPSTATSPMVPASSMGDALASI |
| | | SIPGSETTDIEGEPTSSLTAGRKENSTLQEMNSTTESNIILSNVSVGAI |
| | | TEATKMEVPSFDATFIPTPAQSTKFPDIFSVASSRLSNSPPMTISTHM |
| | | TTTQTGSSGATSKIPLALDTSTLETSAGTPSVVTEGFAHSKITTAMN |
| | | NDVKDVSQTNPPFQDEASSPSSQAPVLVTTLPSSVAFTPQWHSTSSP |
| | | VSMSSVLTSSLYKTAGKVDTSLETVTSSPQSMSNTLDDISVTSAATT |
| | | DIETTHPSINTVVTNVGTTGSAFESHSTVSAYPEPSKVTSPNVTTST |
| | | MEDTTISRSIPKSSKTTRTETETTSSLTPKLRETSISQEITSSTETSTVP |
| | | YKELTGATTEVSRTDVTSSSSTSFPGPDQSTVSLDISTETNTRLSTSPI |
| | | MTESAEITITTQTGPHGATSQDTFTMDPSNTTPQAGIHSAMTHGFSQ |
| | | LDVTTLMSRIPQDVSWTSPPSVDKTSSPSSFLSSPAMTTPSLISSTLPE |
| | | DKLSSPMTSLLTSGLVKITDILRTRLEPVTSSLPNFSSTSDKILATSKD |
| | | SKDTKEIFPSINTEETNVKANNSGHESHSPALADSETPKATTQMVIT |
| | | TTVGDPAPSTSMPVHGSSETTNIKREPTYFLTPRLRETSTSQESSFPT |
| | | DTSFLLSKVPTGTITEVSSTGVNSSSKISTPDHDKSTVPPDTFTGEIPR |
| | | VFTSSIKTKSAEMTITTQASPPESASHSTLPLDTSTTLSQGGTHSTVT |
| | | QGFPYSEVTTLMGMGPGNVSWMTTPPVEETSSVSSLMSSPAMTSPS |
| | | PVSSTSPQSIPSSPLPVTALPTSVLVTTTDVLGTTSPESVTSSPPNLSSI |
| | | THERPATYKDTAHTEAAMHHSTNTAVTNVGTSGSGHKSQSSVLAD |
| | | SETSKATPLMSTTSTLGDTSVSTSTPNISQTNQIQTEPTASLSPRLRES |
| | | STSEKTSSTTETNTAFSYVPTGAITQASRTEISSSRTSISDLDRPTIAPD |
| | | ISTGMITRLFTSPIMTKSAEMTVTTQTTTPGATSQGILPWDTSTTLFQ |
| | | GGTHSTVSQGFPHSEITTLRSRTPGDVSWMTTPPVEETSSGFSLMSP |
| | | SMTSPSPVSSTSPESIPSSPLPVTALLTSVLVTTTNVLGTTSPEPVTSS |
| | | PPNLSSPTQERLTTYKDTAHTEAMHASMHTNTAVANVGTSISGHES |
| | | QSSVPADSHTSKATSPMGITFAMGDTSVSTSTPAFFETRIQTESTSSL |
| | | IPGLRDTRTSEEINTVTETSTVLSEVPTTTTTEVSRTEVITSSRTTISGP |
| | | DHSKMSPYISTETITRLSTFPFVTGSTEMAITNQTGPIGTISQATLTLD |
| | | TSSTASWEGTHSPVTQRFPHSEETTTMSRSTKGVSWQSPPSVEETSS |
| | | PSSPVPLPAITSHSSLYSAVSGSSPTSALPVTSLLTSGRRKTIDMLDT |
| | | HSELVTSSLPSASSFSGEILTSEASTNTETIHFSENTAETNMGTTNSM |
| | | HKLHSSVSIHSQPSGHTPPKVTGSMMEDAIVSTSTPGSPETKNVDRD |
| | | STSPLTPELKEDSTALVMNSTTESNTVFSSVSLDAATEVSRAEVTYY |
| | | DPTFMPASAQSTKSPDISPEASSSHSNSPPLTISTHKTIATQTGPSGVT |
| | | SLGQLTLDTSTIATSAGTPSARTQDFVDSETTSVMNNDLNDVLKTS |
| | | PFSAFEANSLSSQAPLLVTTSPSPVTSTLQEHSTSSLVSVTSVPTPTL |
| | | AKITDMDTNLEPVTRSPQNLRNTLATSEATTDTHTMHPSINTAVAN |
| | | VGTTSSPNEFYFTVSPDSDPYKATSAVVITSTSGDSIVSTSMPRSSAM |
| | | KKIESETTFSLIFRLRETSTSQKIGSSSDTSTVFDKAFTAATTEVSRTE |
| | | LTSSSRTSIQGTEKPTMSPDTSTRSVTMLSTEAGLTKSEERTIATQTG |
| | | PHRATSQGTLTWDTSITTSQAGTHSAMTHGFSQLDLSTLTSRVPEYI |
| | | SGTSPPSVEKTSSSSSLLSLPAITSPSPVPTTLPESRPSSPVHLTSLPTS |
| | | GLVKTTDMLASVASLPPNLGSTSHKIPTTSEDIKDTEKMYPSTNIAV |
| | | TNVGTTTSEKESYSSVPAYSEPPKVTSPMVTSFNIRDTIVSTSMPGSS |
| | | EITRIEMESTFSLAHGLKGTSTSQDPIVSTEKSAVLHKLTTGATETSR |
| | | TEVASSRRTSIPGPDHSTESPDISTEVIPSLPISLGITESSNMTIITRTGP |
| | | PLGSTSQGTFTLDTPTTSSRAGTHSMATQEFPHSEMTTVMNKDPEIL |
| | | SWTIPPSIEKTSFSSSLMPSPAMTSPPVSSTLPKTIHTTPSPMTSLLTPS |
| | | LVMTTDTLGTSPEPTTSSPPNLSSTSHEILTTDEDTTAIEAMHPSTST |
| | | AATNVETTSSGHGSQSSVLADSEKTKATAPMDTTSTMGHTTVSTS |
| | | MSVSSETTKIKRESTYSLTPGLRETSISQNASFSTDTSIVLSEVPTGTT |
| | | AEVSRTEVTSSGRTSIPGPSQSTVLPEISTRTMTRLFASPTMTESAEM |
| | | TIPTQTGPSGSTSQDTLTLDTSTTKSQAKTHSTLTQRFPHSEMTTLM |
| | | SRGPGDMSWQSSPSLENPSSLPSLLSLPATTSPPPISSTLPVTISSSPLP |
| | | VTSLLTSSPVTTTDMLHTSPELVTSSPPKLSHTSDERLTTGKDTTNT |
| | | EAVHPSTNTAASNVEIPSSGHESPSSALADSETSKATSPMFITSTQED |
| | | TTVAISTPHFLETSRIQKESISSLSPKLRETGSSVETSSAIETSAVLSEV |
| | | SIGATTEISRTEVTSSSRTSISGSAESTMLPEISTTRKIIKFPTSPILAESS |
| | | EMTIKTQTSPPGSTSESTFTLDTSTTPSLVITHSTMTQRLPHSEITTLV |
| | | SRGAGDVPRPSSLPVEETSPPSSQLSLSAMISPSPVSSTLPASSHSSSA |
| | | SVTSLLTPGQVKTTEVLDASAEPETSSPPSLSSTSVEILATSEVTTDT |
| | | EKIHPFSNTAVTKVGTSSSGHESPSSVLPDSETTKATSAMGTISIMGD |
| | | TSVSTLTPALSNTRKIQSEPASSLTTRLRETSTSEETSLATEANTVLS |
| | | KYSTGATTEVSRTEAISFSRTSMSGPEQSTMSQDISIGTIPRISASSVL |
| | | TESAKMTITTQTGPSESTLESTLNLNTATTPSWVETHSIVIQGFPHPE |
| | | MTTSMGRGPGGVSWPSPPFVKETSPPSSPLSLPAVTSPHPVSTTFLA |
| | | HIPPSPLPVTSLLTSGPATTTDILGTSTEPGTSSSSSLSTTSHERLTTYK |
| | | DTAHTEAVHPSTNTGGTNVATTSSGYKSQSSVLADSSPMCTTSTM |
| | | GDTSVLTSTPAFLETRRIQTELASSLTPGLRESSGSEGTSSGTKMSTV |
| | | LSKVPTGATTEISKEDVTSIPGPAQSTISPDISTRTVSWFSTSPVMTES |
| | | AEITMNTHTSPLGATTQGTSTLDTSSTTSLTMTHSTISQGFSHSQMS |
| | | TLMRRGPEDVSWMSPPLLEKTRPSFSLMSSPATTSPSPVSSTLPESIS |
| | | SSPLPVTSLLTSGLAKTTDMLHKSSEPVTNSPANLSSTSVEILATSEV |
| | | TTDTEKTHPSSNRTVTDVGTSSSGHESTSFVLADSQTSKVTSPMVIT |
| | | STMEDTSVSTSTPGFFETSRIQTEPTSSLTLGLRKTSSSEGTSLATEM |
| | | STVLSGVPTGATAEVSRTEVTSSSRTSISGFAQLTVSPETSTETITRLP |
| | | TSSIMTESAEMMIKTQTDPPGSTPESTHTVDISTTPNWVETHSTVTQ |
| | | RFSHSEMTTLVSRSPGDMLWPSQSSVEETSSASSLLSLPATTSPSPVS |
| | | STLVEDFPSASLPVTSLLNPGLVITTDRMGISREPGTSSTSNLSSTSHE |
| | | RLTTLEDTVDTEDMQPSTHTAVTNVRTSISGHESQSSVLSDSETPKA |
| | | TSPMGTTYTMGETSVSISTSDFFETSRIQIEPTSSLTSGLRETSSSERIS |
| | | SATEGSTVLSEVPSGATTEVSRTEVISSRGTSMSGPDQFTISPDISTEA |
| | | ITRLSTSPIMTESAESAITIETGSPGATSEGTLTLDTSTTTFWSGTHST |
| | | ASPGFSHSEMTTLMSRTPGDVPWPSLPSVEEASSVSSSLSSPAMTST |
| | | SFFSTLPESISSSPHPVTALLTLGPVKTTDMLRTSSEPETSSPPNLSSTS |
| | | AEILATSEVTKDREKIHPSSNTPVVNVGTVIYKHLSPSSVLADLVTT |
| | | KPTSPMATTSTLGNTSVSTSTPAFPETMMTQPTSSLTSGLREISTSQE |
| | | TSSATERSASLSGMPTGATTKVSRTEALSLGRTSTPGPAQSTISPEIS |
| | | TETITRISTPLTTTGSAEMTITPKTGHSGASSQGTFTLDTSSRASWPG |
| | | THSAATHRSPHSGMTTPMSRQPEDVSWPSRPSVEKTSPPSSLVSLSA |
| | | VTSPSPLYSTPSESSHSSPLRVTSLFTPVMMKTTDMLDTSLEPVTTSP |
| | | PSMNITSDESLATSKATMETEAIQLSENTAVTQMGTISARQEFYSSY |
| | | PGLPEPSKVTSPVVTSSTIKDIVSTTIPASSEITRIEMESTSTLTPTPRET |
| | | STSQEIHSATKPSTVPYKALTSATIEDSMTQVMSSSRGPSPDQSTMS |
| | | QDISTEVITRLSTSPIKTESTEMTITTQTGSPGATSRGTLTLDTSTTFM |
| | | SGTHSTASQGFSHSQMTALMSRTPGDVPWLSHPSVEEASSASFSLSS |
| | | PVMTSSSPVSSTLPDSIHSSSLPVTSLLTSGLVKTTELLGTSSEPETSS |
| | | PPNLSSTSAEILAITEVTTDTEKLEMTNVVTSGYTHESPSSVLADSVT |
| | | TKATSSMGITYPTGDTNVLTSTPAFSDTSRIQTKSKLSLTPGLMETSI |
| | | SEETSSATEKSTVLSSVPTGATTEVSRTEAISSSRTSIPGPAQSTMSSD |
| | | TSMETITRISTPLIRKESTDMAITPKTGPSGATSQGTFTLDSSSTASW |
| | | PGTHSATTQRFPQSVVTTPMSRGPEDVSWPSPLSVEKNSPPSSLVSS |
| | | SSVTSPSPLYSTPSGSSHSSPVPVTSLFTSIMMKATDMLDASLEPETT |
| | | SAPNMNITSDESLAASKATTETEAIHVFENTAASHVETTSATEELYS |
| | | SSPGFSEPTKVISPVVTSSSIRDNMVSTTMPGSSGITRIEIESMSSLTPG |
| | | LRETRTSQDITSSTETSTVLYKMPSGATPEVSRTEVMPSSRTSIPGPA |
| | | QSTMSLDISDEVVTRLSTSPIMTESAEITITTQIGYSLATSQVTLPLG |
| | | TSMTFLSGTHSTMSQGLSHSEMTNLMSRGPESLSWTSPRFVETTRS |
| | | SSSLTSLPLTTSLSPVSSTLLDSSPSSPLPVTSLILPGLVKTTEVLDTSS |
| | | EPKTSSSPNLSSTSVEIPATSEIMTDTEKIHPSSNTAVAKVRTSSSVHE |
| | | SHSSVLADSETTITIPSMGITSAVDDTTVFTSNPAFSETRRIPTEPTFSL |
| | | TPGFRETSTSEETTSITETSAVLYGVPTSATTEVSMTEIMSSNRIHIPD |
| | | SDQSTMSPDIITEVITRLSSSSMMSESTQMTITTQKSSPGATAQSTLT |
| | | LATTTAPLARTHSTVPPRFLHSEMTTLMSRSPENPSWKSSLFVEKTS |
| | | SSSSLLSLPVTTSPSVSSTLPQSIPSSSFSVTSLLTPGMVKTTDTSTEPG |
| | | TSLSPNLSGTSVEILAASEVTTDTEKIHPSSSMAVTNVGTTSSGHELY |
| | | SSVSIHSEPSKATYPVGTPSSMAETSISTSMPANFETTGFEAEPFSHL |
| | | TSGFRKTNMSLDTSSVTPTNTPSSPGSTHLLQSSKTDFTSSAKTSSPD |
| | | WPPASQYTEIPVDIITPFNASPSITESTGITSFPESRFTMSVTESTHHLS |
| | | TDLLPSAETISTGTVMPSLSEAMTSFATTGVPRAISGSGSPFSRTESG |
| | | PGDATLSTIAESLPSSTPVPFSSSTFTTTDSSTIPALHEITSSSATPYRV |
| | | DTSLGTESSTTEGRLVMVSTLDTSSQPGRTSSSPILDTRMTESVELG |
| | | TVTSAYQVPSLSTRLTRTDGIMEHITKIPNEAAHRGTIRPVKGPQTST |
| | | SPASPKGLHTGGTKRMETTTTALKTTTTALKTTSRATLTTSVYTPTL |
| | | GTLTPLNASMQMASTIPTEMMITTPYVFPDVPETTSSLATSLGAETS |
| | | TALPRTTPSVFNRESETTASLVSRSGAERSPVIQTLDVSSSEPDTTAS |
| | | WVIHPAETIPTVSKTTPNFFHSELDTVSSTATSHGADVSSAIPTNISPS |
| | | ELDALTPLVTISGTDTSTTFPTLTKSPHETETRTTWLTHPAETSSTIPR |
| | | TIPNFSHHESDATPSIATSPGAETSSAIPIMTVSPGAEDLVTSQVTSSG |
| | | TDRNMTIPTLTLSPGEPKTIASLVTHPEAQTSSAIPTSTISPAVSRLVT |
| | | SMVTSLAAKTSTTNRALTNSPGEPATTVSLVTHPAQTSPTVPWTTSI |
| | | FFHSKSDTTPSMTTSHGAESSSAVPTPTVSTEVPGVVTPLVTSSRAVI |
| | | STTIPILTLSPGEPETTPSMATSHGEEASSAIPTPTVSPGVPGVVTSLV |
| | | TSSRAVTSTTIPILTFSLGEPETTPSMATSHGTEAGSAVPTVLPEVPG |
| | | MVTSLVASSRAVTSTTLPTLTLSPGEPETTPSMATSHGAEASSTVPT |
| | | VSPEVPGVVTSLVTSSSGVNSTSIPTLILSPGELETTPSMATSHGAEA |
| | | SSAVPTPTVSPGVSGYVTPLVTSSRAVTSTTIPILILSSSEPETTPSMA |
| | | TSHGVEASSAVLIVSPEVPQMVTSLVTSSRAVTSTTIPTLTISSDEPE |
| | | TTTSLVTHSEAKMISAIPTLAVSPTVQGLVTSLVTSSGSETSAFSNLT |
| | | VASSQPETIDSWVAHPGTEASSVVPTLTVSTGEPFTNISLVTHPAESS |
| | | STLPRTTSRFSHSELDTMPSTVTSPEAESSSAISTTISPGIPGVLTSLVT |
| | | SSGRDISATFPTVPESPHESEATASWVTHPAVTSTTVPRTTPNYSHSE |
| | | PDTTPSIATSPGAEATSDFPTITVSPDVPDMVTSQVTSSGTDTSITIPT |
| | | LILSSGEPETTTSFITYSETHTSSAIPTLPVSPGASKMLTSLVISSGTDS |
| | | TTTFPTLTETPYEPETTAIQLIHPAETNTMVPRTTPKFSHSKSDTTLP |
| | | VAITSPQPEASSAVSTTTISPDMSDLVTSLVPSSGTDTSTTFPTLSETP |
| | | YEPETTATWLTHPAETSTTVSGTIPNFSHRGSDTAPSMVTSPGVDTR |
| | | SGVPTTTIPPSIPGVVISQVTSSATDTSTAIPTLTPSPGEPETTASSAT |
| | | HPGTQTGFTVPIRTVPSSEPDTMASWVTHPPQTSTPVSRTTSSFSHSS |
| | | PDATPVMATSPRTEASSAVLTTISPGAPEMVTSQITSSGAATSTTVPT |
| | | LTHSPGMPETTALLSTHPRTETSKTFPASTVFPQVSETTASLTIRPGA |
| | | ETSTALPTQTTSSLFTLLVTGTSRVDLSPTASPGVSAKTAPLSTHPGT |
| | | ETSTMIPTSTLSLGLLETTGLLATSSSAETSTSTLTLTVSPAVSGLSSA |
| | | SITTDKPQTVTSWNTETSPSVTSVGPPEFSRTVTGTTMTLIPSEMPTP |
| | | PKTSHGEGVSPTTILRTTMVEATNLATTGSSPTVAKTTTTFNTLAGS |
| | | LFTPLTTPGMSTLASESVTSRTSYNHRSWISTTSSYNRRYWTPATST |
| | | PVTSTFSPGISTSSIPSSTAATVPFMVPFTLNFTITNLQYEEDMRHPGS |
| | | RKFNATERELQGLLKPLFRNSSLEYLYSGCRLASLRPEKDSSATAV |
| | | DAICTHRPDPEDLGLDRERLYWELSNLTNGIQELGPYTLDRNSLYV |
| | | NGFTHRSSMPTTSTPGTSTVDVGTSGTPSSSPSPTTAGPLLMPFTLNF |
| | | TITNLQYEEDMRRTGSRKFNTMESVLQGLLKPLFKNTSVGPLYSGC |
| | | RLTLLRPEKDGAATGVDAICTHRLDPKSPGLNREQLYWELSKLTND |
| | | IEELGPYTLDRNSLYVNGFTHQSSVSTTSTPGTSTVDLRTSGTPSSLS |
| | | SPTIMAAGPLLVPFTLNFTITNLQYGEDMGHPGSRKFNTTERVLQG |
| | | LLGPIFKNTSVGPLYSGCRLTSLRSEKDGAATGVDAICIHHLDPKSP |
| | | GLNRERLYWELSQLTNGIKELGPYTLDRNSLYVNGFTHRTSVPTSS |
| | | TPGTSTVDLGTSGTPFSLPSPATAGPLLVLFTLNFTITNLKYEEDMH |
| | | RPGSRKFNTTERVLQTLLGPMFKNTSVGLLYSGCRLTLLRSEKDGA |
| | | ATGVDAICTHRLDPKSPGVDREQLYWELSQLTNGIKELGPYTLDRN |
| | | SLYVNGFTHWIPVPTSSTPGTSTVDLGSGTPSSLPSPTTAGPLLVPFT |
| | | LNFTITNLKYEEDMHCPGSRKFNTTERVLQSLLGPMFKNTSVGPLY |
| | | SGCRLTLLRSEKDGAATGVDAICTHRLDPKSPGVDREQLYWELSQL |
| | | TNGIKELGPYTLDRNSLYVNGFTHQTSAPNTSTPGTSTVDLGTSGTP |
| | | SSLPSPTSAGPLLVPFTLNFTITNLQYEEDMHHPGSRKFNTTERVLQ |
| | | GLLGPMFKNTSVGLLYSGCRLTLLRPEKNGAATGMDAICSHRLDP |
| | | KSPGLNREQLYWELSQLTHGIKELGPYTLDRNSLYVNGFTHRSSVA |
| | | PTSTPGTSTVDLGTSGTPSSLPSPTTAVPLLVPFTLNFTITNLQYGED |
| | | MRHPGSRKFNTTERVLQGLLQPLFKNSSVGPLYSGCRLISLRSEKD |
| | | GAATGVDAICTHHLNPQSPGLDREQLYWQLSQMTNGIKELGPYTL |
| | | DRNSLYVNGFTHRSSGLTTSTPWTSTVDLGTSGTPSPVPSPTTTGPL |
| | | LVPFTLNFTITNLQYEENMGHPGSRKFNITESVLQGLLKPLFKSTSV |
| | | GPLYSGCRLTLLRPEKDGVATRVDAICTHRPDPKIPGLDRQQLYWE |
| | | LSQLTHSITELGPYTLDRDSLYVNGFTQRSSVPTTSTPGTFTVQPETS |
| | | ETPSSLPGPTATGPVLLPFTLNFTITNLQYEEDMRRPGSRKFNTTER |
| | | VLQGLLMPLFKNTSVSSLYSGCRLTLLRPEKDGAATRVDAVCTHRP |
| | | DPKSPQLDRERLYWKLSQLTHGITELGPYTLDRHSLYVNGFTHQSS |
| | | MTTTRTPDTSTMHLATSRTPASLSGPMTASPLLVLFTINFTITNLRYE |
| | | ENMHHPGSRKFNTTERVLQGLLRPVFKNTSVGPLYSGCRLTLLRPK |
| | | KDGAATKVDAICTYRPDPKSPGLDREQLYWELSQLTHSITELGPYT |
| | | LDRDSLYVNGFTQRSSVPTTSIPGTPTVDLGTSGTPVSKPGPSAASPL |
| | | LVLFTLNFTITNLRYEENMQHPGSRKFNTTERVLQGLLRSLFKSTSV |
| | | GPLYSGCRLTLLRPEKDGTATGVDAICTHHPDPKSPRLDREQLYWE |
| | | LSQLTHNITELQPYALDNDSLFVNQFTHRSSVSTTSTPGTPTVYLGA |
| | | SKTPASIFGPSAASHLLILFTLNFTITNLRYEENMWPGSRKFNTTERV |
| | | LQGLLRPLFKNTSVGPLYSGCRLTLLRPEKDGEATGVDAICTHRPD |
| | | PTGPGLDREQLYLELSQLTHSITELQPYTLDRDSLYVNGFTHRSSVP |
| | | TTSTGVVSEEPFTLNFTINNLRYMADMGQPGSLKFNITDNVMQHLL |
| | | SPLFQRSSLGARYTGCRVIALRSVKNGAETRVDLLCTYLQPLSGPG |
| | | LPIKQVFHELSQQTHGITRLGPYSLDKDSLYLNGYNEPQPDEPPTTP |
| | | KPATTFLPPLSEATTAMGYHLKTLTLNFTISNLQYSPDMGKGSATF |
| | | NSTEGVLQHLLRPLFQKSSMGPFYLGCQLISLRPEKDGAATGVDTT |
| | | CTYHPDPVGPGLDIQQLYWELSQLTHGVTQLGFYVLDRDSLFINGY |
| | | APQNLSIRGEYQINFHIVNWNLSNPDPTSSEYITLLRDIQDKVTTLYK |
| | | GSQLHDTFRFCLVTNLTMDSVLVTVKALFSSNLDPSLVEQVFLDKT |
| | | LNASFHWLGSTYQLVDIHVTEMESSVYQPTSSSSTQHFYLNFTITNL |
| | | PYSQDKAQPGTTNYQRNKRNIEDALNQLFRNSSIKSYFSDCQVSTF |
| | | RSVPNRHHTGVDSLCNFSPLARRVDRVAIYEEFLRMTRNGTQLQNF |
| | | TLDRSSVLVDGYSPNRNEPLTGNSDLPFWAVILIGLAGLLGVITCLI |
| | | CGVLVTTRRRKKEGEYNVQQQCPGYYQSHLDLEDLQ |
| 2 | Clone 8 | QVQLQQWGAGLLKPSETLSLTCAVYGGSFSGYYWSWIRQPPQKGL |
| | VH | EWIGEINHSGSTNYNPSLKSRVTISVDTSKNQFSLKLSSVTAADTAV |
| | | YYCARQSYITDSWGQGTLVTVSS |
| 3 | Clone 8 VL | DIQLTQSPSAVSASVGDRVTITCRASQDVSKWLAWYQQKPGKAPR |
| | | LLISAASGLQSWVPSRFSGSGSGTEFTLSISSLQPEDFATYYCQQANS |
| | | FPWTFGQGTKVEIKR |
| 4 | Clone 8 | GGSFSGYY |
| | HC-CDR1 | |
| 5 | Clone 8 | INHSGST |
| | HC-CDR2 | |
| 6 | Clone 8 | ARQSYITDS |
| | HC-CDR3 | |
| 7 | Clone 8 | QDVSKW |
| | LC-CDR1 | |
| 8 | Clone 8 | AAS |
| | LC-CDR2 | |
| 9 | Clone 8 | QQANSFPWT |
| | LC-CDR3 | |
| 10 | Clone 12 | QVQLQQWGAGLLKPSETLSLTCAVYGGSFSGYYWSWIRQPPGKGL |
| | VH | EWIGEINHSGSTNYNPSLKSRIIMSVDTSKRQFSLKLRSATAADTAV |
| | | YYCARWSPFSYKQMYDYWGQGTLVTVSS |
| 11 | Clone 12 | NFMLTQPHSVSESPGKTVTISCTRSRGSIASAYVQWYQQRPGSAPIT |
| | VL | VIYEDYERPSEIPDRFSGSIDSSSNSASLTISGLKTEDEADYYCQSYD |
| | | DNDHVIFGGGTKYTVLG |
| 12 | Clone 12 | GGSFSGYY |
| | HC-CDR1 | |
| 13 | Clone 12 | INHSGST |
| | HC-CDR2 | |
| 14 | Clone 12 | ARWSPFSYKQMYDY |
| | HC-CDR3 | |
| 15 | Clone 12 | RGSIASAY |
| | LC-CDR1 | |
| 16 | Clone 12 | EDY |
| | LC-CDR2 | |
| 17 | Clone 12 | QSYDDNDHVI |
| | LC-CDR3 | |
| 18 | Signal | METDTLLLWVLLLWVPGSTG |
| | peptide | |
| 19 | Clone 8 | DIQLTQSPSAVSASVGDRVTITCRASQDVSKWLAWYQQKPGKAPR |
| | scFv | LLISAASGLQSWVPSRFSGSGSGTEFTLSISSLQPEDFATYYCQQANS |
| | | FPWTFGQGTKVEIKRSRGGGGSGGGGSGGGGSLEMAQVQLQQWG |
| | | AGLLKPSETLSLTCAVYGGSFSGYYWSWIRQPPGKGLEWIGEINHS |
| | | GSTNYNPSLKSRVTISVDTSKNQFSLKLSSVTAADTAVYYCARQSYI |
| | | TDSWGQGTLVTVSS |
| 20 | Clone 12 | NFMLTQPHSVSESPGKTVTISCTRSRGSIASAYVQWYQQRPGSAPIT |
| | scFv | VIYEDYERPSEIPDRFSGSIDSSSNSASLTISGLKTEDEADYYCQSYD |
| | | DNDHVIFGGGTKVTVLGSRGGGGSGGGGSGGGGSLEMAQVQLQQ |
| | | WGAGLLKPSETLSLTCAVYGGSFSGYYWSWIRQPPGKGLEWIGEIN |
| | | HSGSTNYNPSLKSRIIMSVDTSKRQFSLKLRSATAADTAVYYCARW |
| | | SPFSYKQMYDYWGQGTLVTVSS |
| 21 | Anti-CD3 | DVQLVQSGAEVKKPGASVKVSCKASGYTFTRYTMHWVRQAPGQG |
| | scFv | LEWIGYINPSRGYTNYADSVKGRFTITTDKSTSTAYMELSSLRSEDT |
| | | ATYYCARYYDDHYCLDYWGQGTTVTVSSGEGTSTGSGGSGGSGG |
| | | ADDIVLTQSPATLSLSPGERATLSCRASQSVSYMNWYQQKPGKAP |
| | | KRWIYDTSKVASGYPARFSGSGSGTDYSLTINSLEAEDAATYYCQQ |
| | | WSSNPLTFGGQTKVEIK |
| 22 | Clone | METDTLLLWVLLLWVPGSTGDIQLTQSPSAVSASVGDRVTITCRAS |
| | 8/anti-CD3 | QDVSKWLAWYQQKPGKAPRLLISAASGLQSWVPSRFSGSGSGTEF |
| | bispecific | TLSISSLQPEDFATYYCQQANSFPWTFGQGTKVEIKRSRGGGGSGG |
| | antibody | GGSGGGGSLEMAQVQLQQWGAGLLKPSETLSLTCAVYGGSFSGY |
| | | YWSWIRQPPGKGLEWIGEINHSGSTNYNPSLKSRVTISVDTSKNQFS |
| | | LKLSSVTAADTAVYYCARQSYITDSWGQGTLVTVSSTSGGGGSDV |
| | | QLVQSGAEVKKPGASVKVSCKASGYTFTRYTMHWVRQAPGQGLE |
| | | WIGYINPSRGYTNYADSVKGRFTITTDKSTSTAYMELSSLRSEDTAT |
| | | YYCARYYDDHYCLDYWGQGTTVTVSSGEGTSTGSGGSGGSGGAD |
| | | DIVLTQSPATLSLSPGERATLSCRASQSVSYMNWYQQKPGKAPKR |
| | | WIYDTSKVASGVPARFSGSGSGTDYSLTINSLEAEDAATYYCQQWS |
| | | SNPLTFGGGTKVEIKHHHHHH |
| 23 | Clone | METDTLLLWVLLLWVPGSTGNFMLTQPHSVSESPGKTVTISCTRSR |
| | 12/anti- | GSIASAYVQWYQQRPGSAPITVIYEDYERPSEIPDRFSGSIDSSSNSA |
| | CD3 | SLTISGLKTEDEADYYCQSYDDNDHVIFGGGTKVTVLGSRGGGGS |
| | bispecific | GGGGSGGGGSLEMAQVQLQQWGAGLLKPSETLSLTCAVYGGSFS |
| | antibody | GYYWSWIRQPPGKGLEWIGEINHSGSTNYNPSLKSRIIMSVDTSKR |
| | | QFSLKLRSATAADTAVYYCARWSPFSYKQMYDYWGQGTLVTVSS |
| | | TSGGGGSDVQLVQSGAEVKKPGASVKVSCKASGYTFTRYTMHWV |
| | | RQAPGQGLEWIGYINPSRGYTNYADSVKGRFTITTDKSTSTAYMEL |
| | | SSLRSEDTATYYCARYYDDHYCLDYWGQGTTVTVSSGEGTSTGSG |
| | | GSGGSGGADDIVLTQSPATLSLSPGERATLSCRASQSVSYMNWYQ |
| | | QKPGKAPKRWIYDISKVASGVPARFSGSGSGTDYSLTINSLEAEDA |
| | | ATYYCQQWSSNPLTFGGGTKVEIKHHHHHH |
| 24 | MUC16c344 | WELSQLTHGVTQLGFYVLDRDSLFINGYAPQNLSIRGEYQINFHIVN |
| | | QNLSNPDPTSSEYITLLRDIQDKVTTLYKGSQLHDTFRFCLVTNLTM |
| | | DSVLVTVKALFSSNLDPSLVEQVFLDKTLNASFHQLGSTYQLVDIH |
| | | VTEMESSVYQPTSSSSTQHFYLNFTITNLPYSQDKAQPGTTNYQRN |
| | | KRNIEDALNQLFRNSSIKSYFSDCQVSTFRSVPNRHHTGVDSLCNFS |
| | | PLARRVDRVAIYEEFLRMTRNGTQLQNFTLDRSSVLVDGYSPNRNE |
| | | PLIGNSDLPFWAVILIGLAGLLGLITCLICGVLVTTRRRKKEGEYNV |
| | | QQQCPGYYQSHLDLEDLQ |
| 25 | MUC16c114 | NFSPLARRVDRVAIYEEFLRMTRNGTQLQNFTLDRSSVLVDGYSPN |
| | | RNEPLTGNSDLPFWAVILIGLAGLLGLITCLICGVLVTTRRRKKEGE |
| | | YNVQQQCPGYYQSHLDLEDLQ |
| 26 | MUC16c86 | NFSPLARRVDRVAIYEEFLRMDLPFWAVILIGLAQLLGLITCLICGV |
| | | LVTTRRRKKEGEYNVQQQCPGYYQSHLDLEDLQ |
| 27 | MUC16c80 | NFSPLARRVDRVAIYEEFLRMTRNGTQLQNFTLDRSSVLVDGYSPN |
| | | RNEPLTGNSDLPFWAVILIGLAGLLGLITCLICGDLEDLQ |
| 28 | IgG1 heavy | ASTKGPSVFPLAPSSKSTSGGTAALGCLVKDYFPEPVTVSWNSGAL |
| | chain | TSGVHTFPAVLQSSGLYSLSSVVTVPSSSLGTQTYICNVNHKPSNTK |
| | constant | VDKKVEPKSCDKTHTCPPCPAPELLGGPSVFLFPPKPKDTLMISRTP |
| | region | EVTCVVVDVSHEDPEVKFNWYVDGVEVHNAKTKPREEQYNSTYR |
| | | VVSVLTVLHQDWLNGKEYKCKVSNKALPAPIEKTISKAKGQPREP |
| | | QVYTLPPSREEMTKNQVSLTCLVKGFYPSDIAVEWESNGQPENNY |
| | | KTTPPVLDSDGSFFLYSKLTVDKSRWQQGNVFSCSVMHEALHNHY |
| | | TQKSLSLSPGK |
| 29 | IgG4 heavy | ASTKGPSVFPLAPCSRSTSESTAALGCLVKDYFPEPVTVSWNSGALT |
| | chain | SGVHTFPAVLQSSGLYSLSSVVTVPSSSLGTKTYTCNVDHKPSNTK |
| | constant | VDKRVESKYGPPCPSCPAPEFLGGPSVFLFPPKPKDTLMISRTPEVT |
| | region | CVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSL |
| | | SLSLGK |
| 30 | Light chain | QPKANPTVTLFPPSSEELQANKATLVCLISDFYPGAVTVAWKADGS |
| | constant | PVKAGVETTKPSKQSNNKYAASSYLSLTPEQWKSHRSYSCQVTHE |
| | region | GSTVEKTVAPTECS |
| 31 | MUC16c114 | NFSPLARRVDRVAIYEEFLRMIRNGTQLQAFTLDRSSVLVDGYSPN |
| | N30A | RNEPLTGNSDLPFWAVILIGLAGLLGLITCLICGVLVTTRRRKKEGE |
| | | YNVQQQCPGYYQSHLDLEDLQ |
| | hMUC16 | DKTLASPTSSVVGRTTQSLGVMSSALPESTSRGMTHSEQRTSPSLSP |
| 32 | (mature) | QVNGTPSRNYPATSMVSGLSSPRTRTSSTEGNFTKEASTYTLTVETT |
| | | SGPVTEKYTVPTETSTTEGDSTETPWDTRYIPVKITSPMKTFADSTA |
| | | SKENAPVSMTPAETTVTDSHTPGRTNPSFGTLYSSFLDLSPKGTPNS |
| | | RGETSLELILSTTGYPFSSPEPGSAGHSRISTSAPLSSSASVLDNKISET |
| | | SIFSGQSLTSPLSPGVPEARASTMPNSAIPFSMTLSNAETSAERVRSTI |
| | | SSLGTPSISTKQTAETILTFHAFAETMDIPSTHIAKTLASEWLGSPGT |
| | | LGGTSTSALTTTSPSTTLVSEETNTHHSTSGKETEGTLNTSMTPLETS |
| | | APGEESEMTATLVPTLGFTTLDSKIRSPSQVSSSHPTRELRTTGSTSG |
| | | RQSSSTAAHGSSDILRATTSSTSKASSWTSESTAQQFSEPQHTQWVE |
| | | TSPSMKTERPPASTSVAAPITTSVPSVVSGFTTLKTSSTKGIWLEETS |
| | | ADTLIGESTAGPTTHQFAVPTGISMTQGSSTRGSQGTTHLLTRATAS |
| | | SETSADLTLATNGVPVSVSPAVSKTAAGSSPPGGTKPSYTMVSSVIP |
| | | ETSSLQSSAFREGTSLGLTPLNTRHPFSSPEPDSAGHTKISTSIPLLSS |
| | | ASVLEDKVSATSTFSHHKATSSITTGTPEISTKTKPSSAVLSSMTLSN |
| | | AATSPERVRNATSPLTHPSPSGEETAGSVLTLSTSAETTDSPNIHPTG |
| | | TLTSESSESPSTLSLPSVSGVKTTFSSSTPSTHLFTSGEETEETSNPSVS |
| | | QPETSVSRVRTTLASTSVPTPVFPTMDTWPTRSAQFSSSHLVSELRA |
| | | TSSTSVTNSTGSALPKISHLTGTATMSQTNRDTFNDSAAPQSTTWPE |
| | | TSPRFKTGLPSATTTVSTSATSLSATVMVSKFTSPATSSMEATSIREP |
| | | STTILTTETTNGPGSMAVASTNIPICKGYITEGRLDTSHLPIGTTASSE |
| | | TSMDFTMAKESVSMSVSPSQSMDAAGSSTPQRTSQFVDTFSDDVY |
| | | HLTSREITIPRDGTSSALTPQMTATHPPSPDPGSARSTWLGILSSSPSS |
| | | PTPKVTMSSTFSTQRVTTSMIMDTVETSRWNMPNLPSTTSLIPSNIP |
| | | TSGAIGKSTLVPLDTPSPATSLEASEGGLPTLSTYPESTNTPSIHLGA |
| | | HASSESPSTIKLTMASVVKPGSYTPLTFPSIETHIHVSTARMAYSSGS |
| | | SPEMTAPGETNTGSTWDPTTYITTTDPKDTSSAQVSTPHSVRTLRTT |
| | | ENHPKTESATPAAYSGSPKISSSPNLTSPATKAWTITDTTEHSTQLH |
| | | YTKLAEKSSGFETQSAPGPVSVVIPTSPTIGSSTLELTSDVPGEPLVL |
| | | APSEQTTITLPMATWLSTSLTEEMASTDLDISSPSSPMSTFAIFPPMS |
| | | TPSHELSKSEADISAIRNTDSITLDQHLGIRSLGRTGDLTTVPITPLT |
| | | TTWTSVIEHSTQAQDTLSATMSPTHYTQSLKDQTSIPASASPSHLTE |
| | | VYPELGTQGRSSSEATTFWKPSTDTLSREIETGPTNIQSTPPMDNTTT |
| | | GSSSSGVTLGIAHLPIGTSSPAETSTNMALERRSSTATVSMAGTMGL |
| | | LVTSAPGRSISQSLGRVSSVLSESTTEGVTDSSKGSSPRLNTQGNTA |
| | | LSSSLEPSYAEGSQMSTSIPLTSSPTTPDVEFIGGSTFWTKEVTTVMT |
| | | SDISKSSARTESSSATLMSTALGSTENIGKEKLRTASMDLPSPTPSM |
| | | EVTPWISLTLSNAPNTTDSLDLSHGVHTSSAGTLATDRSLNTGVTR |
| | | ASRLENGSDTSSKSLSMGNSTHTSMTYTEKSEVSSSIHPRPETSAPG |
| | | AETTLTSTPGNRAISLTLPFSSIPVEEVISTGITSGPDINSAPMTHSPITP |
| | | PTIVWTSTGTIEQSTQPLHAVSSEKVSVQTQSTPYVNSVAVSASPTH |
| | | ENSVSSGSSTSSPYSSASLESLDSTISRRNAITSWLWDLTTSLPTTTW |
| | | PSTSLSEALSSGHSGVSNPSSTTTEFPLFSAASTSAAKQRNPETETHG |
| | | PQNTAASTLNTDASSVTGLSETPVGASISSEVPLPMAITSRSDVSGLT |
| | | SESTANPSLGTASSAGTKLTRTISLPTSESLVSFRMNKDPWTVSIPLG |
| | | SHPTTNTETSIPVNSAGPPGLSTVASDVIDTPSDGAESIPTVSFSPSPD |
| | | TEVTTISHFPEKTTHSFRTISSLTHELTSRVTPIPQDWMSSAMSTKPT |
| | | GASPSITLGERRTITSAAPTTSPIVLTASFTETSTVSLDNETTVKTSDI |
| | | LDARKTNELPSDSSSSSDLINTSIASSTMDVTKTASISPTSISGMTASS |
| | | SPSLFSSDRPQVPTSTTETNTATSPSVSSNTYSLDGGSNYGGTPSTLP |
| | | PFTITHPVETSSALLAWSRPVRTFSTMVSTDTASGENPTSSNSVVTS |
| | | VPAPGTWTSVGSTTDLPAMGFLKTSPAGEAHSLLASTIEPATAFTPH |
| | | LSAAVVTGSSATSEASLLTTSESKAIHSSPQTPTTPTSGANWETSATP |
| | | ESLLVVTETSDTTLTSKILVTDTILFSTVSTPPSKFPSTGTLSGASFPT |
| | | LLPDTPAIPLTATEPTSSLATSFDSTPLVTIASDSLGTVPETTLTMSET |
| | | SNGDALVLKTVSNPDRSIPGITIQGVTESPLHPSSTSPSKIVAPRNTTY |
| | | EGSITVALSTLPAGTTGSLVFSQSSENSETTALVDSSAGLERASVMP |
| | | LTTGSQGMASSGGIRSGSTHSTGTKTFSSLPLTMNPGEVTAMSEITT |
| | | NRLTATQSTAPKGIPVKPTSAESGLLTPVSASSSPSKAFASLTTAPPT |
| | | WGIPQSTLTFEFSEVPSLDTKSASLPTPGQSLNTIPDSDASTASSSLSK |
| | | SPEKNPRARMMTSTKAISASSFQSTGFTETPEGSASPSMAGHEPRVP |
| | | TSGTGDPRYASESMSYPDPSKASSAMTSTSLASKLITLFSTGQAARS |
| | | GSSSSPISLSTEKETSFLSPTASTSRKTSLFLGPSMARQPNILVHLQTS |
| | | ALTLSPTSTLNMSQEEPPELTSSQTIAEEEGTTAETQTLTFTPSETPTS |
| | | LLPVSSPTEPTARRKSSPETWASSISVPAKTSLVETTDGTLVTTIKMS |
| | | SQAAQGNSTWPAPAEETGSSPAGTSPGSPEMSTTLKIMSSKEPSISPE |
| | | IRSTVRNSPWKTPETTVPMETTVEPVTLQSTALGSGSTSISHLPTGTT |
| | | SPTKSPTENMLATERVSLSPSPPEAWTNLYSGTPGGTRQSLATMSS |
| | | VSLESPTARSITGTGQQSSPELVSKTTGMEFSMWHGSTGGTTGDTH |
| | | VSLSTSSNILEDPVTSPNSVSSLTDKSKHKTETWVSTTAIPSTVLNNK |
| | | IMAAEQQTSRSVDEAYSSTSSWSDQTSGSDITLGASPDVTNTLYITS |
| | | TAQTTSLVSLPSGDQGITSLTNPSGGKTSSASSVTSPSIGLETLRANV |
| | | SAVKSDIAPTAGHLSQTSSPAEVSILDVTTAPTPGISTTITTMGTNSIS |
| | | TTTPNPEVGMSTMDSTPATERRTTSTEHPSTWSSTAASDSWTVTDM |
| | | TSNLKVARSPGTISTMHTTSFLASSTELDSMSTPHGRITVIGTSLVTP |
| | | SSDASAVKTETSTSERTLSPSDTTASTPISTFSRVQRMSISVPDILSTS |
| | | WTPSSTEAEDVPVSMVSTDHASTKTDPNTPLSTFLFDSLSTLDWDT |
| | | GRSLSSATATTSAPQGATTPQELTLETMISPATSQLPFSIGHITSAVTP |
| | | AAMARSSGVTFSRPDPTSKKAEQTSTQLPTTTSAHPGQVPRSAATT |
| | | LDVIPHTAKTPDATFQRQGQTALTTEARATSDSWNEKEKSTPSAPW |
| | | ITEMMNSVSEDTIKEVTSSSSVLRTLNTLDINLESGTTSSPSWKSSPY |
| | | ERIAPSESTTDKEAIHPSTNTVETTGWVTSSEHASHSTIPAHSASSKL |
| | | TSPVVTTSTREQAIVSMSTTTWPESTRARTEPNSFLTIELRDVSPYM |
| | | DTSSTTQTSIISSPQSTAITKGPRTEITSSKRISSSFLAQSMRSSDSPSE |
| | | AITRLSNFPAMTESGGMILAMQTSPPGATSLSAPTLDTSATASWTGT |
| | | PLATTQRFTYSEKTTLFSKGPEDTSQPSPPSVEETSSSSSLVPIHATTS |
| | | PSNILLTSQGHSPSSTPPVTSVFLSETSGLGKTTDMSRISLEPGTSLPP |
| | | NLSSTAGEALSTYEASRDTKAIHHSADTAVTNMEATSSEYSPIPGHT |
| | | KPSKATSPLVTSHIMGDITSSTSVFGSSETTEIETVSSVNQGLQERSTS |
| | | QVASSATETSTVITHVSSGDATTHVTKTQATFSSGTSISSPHQFITST |
| | | NTFTDVSTNPSTSLIMTESSGVTITTQTGPTGAATQGPYLLDTSTMP |
| | | YLTETPLAVTPDFMQSEKTTLISKGPKDVSWTSPPSVAETSYPSSLT |
| | | PFLVTTIPPATSTLQGQHTSSPVSATSVLTSGLVKTTDMLNTSMEPV |
| | | TNSPQNLNNPSNEILATLAATTDIETIHPSINKAVTNMGTASSAHVL |
| | | HSTLPVSSEPSTATSPMVPASSMGDALASISIPGSETTDIEGEPTSSLT |
| | | AGRKENSTLQEMNSTTESNIILSNVSVGAITEATKMEVPSFDATFIPT |
| | | PAQSTKFPDIFSVASSRLSNSPPMTISTHMTTTQTGSSGATSKIPLAL |
| | | DTSTLETSAGTPSVVTEGFAHSKITTAMNNDVKDVSQTNPPFQDEA |
| | | SSPSSQAPVLVTTLPSSVAFTPQWHSTSSPVSMSSVLTSSLVKTAGK |
| | | VDTSLETVTSSPQSMSNTLDDISVTSAATTDIETTHPSINTVVTNVGT |
| | | TGSAFESHSTVSAYPEPSKVTSPNVTTSTMEDTTISRSIPKSSKTTRT |
| | | ETETTSSLTPKLRETSISQEITSSTETSTVPYKELTGATTEVSRTDVTS |
| | | SSSTSFPGPDQSTVSLDISTETNTRLSTSPIMTESAEITITTQTGPHGAT |
| | | SQDTFTMDPSNTTPQAGIHSAMTHGFSQLDVTTLMSRIPQDVSWTS |
| | | PPSVDKTSSPSSFLSSPAMTTPSLISSTLPEDKLSSPMTSLLTSQLVKI |
| | | TDILRTRLEPVTSSLPNFSSTSDKILATSKDSKDTKEIFPSINTEETNV |
| | | KANNSGHESHSPALADSETPKATTQMVITTTVGDPAPSTSMPVHGS |
| | | SETTNIKREPTYFLTPRLRETSTSQESSFPTDTSFLLSKVPTGTITEVSS |
| | | TQVNSSSKISTPDHDKSTVPPDTFTGEIPRVFTSSIKTKSAEMTITTQA |
| | | SPPESASHSTLPLDTSTTLSQGGTHSTVTQGFPYSEVTTLMGMGPGN |
| | | VSWMTTPPVEETSSVSSLMSSPAMTSPSPVSSTSPQSIPSSPLPVTAL |
| | | PTSVLVTTTDVLGTTSPESVTSSPPNLSSITHERPATYKDTAHTEAA |
| | | MHHSTNTAVTNVGTSGSGHKSQSSVLADSETSKATPLMSTTSTLGD |
| | | TSVSTSTPNISQTNQIQTEPTASLSPRLRESSTSEKTSSTTETNTAFSY |
| | | VPTGAITQASRTEISSSRTSISDLDRPTIAPDISTGMITRLFTSPIMTKS |
| | | AEMTVTTQTTTPGATSQGILPWDTSTTLFQGGTHSTVSQGFPHSEIT |
| | | TLRSRTPGDVSWMTTPPVEETSSGFSLMSPSMTSPSPVSSTSPESIPSS |
| | | PLPVTALLTSVLVTTTNVLGTTSPEPVTSSPPNLSSPTQERLTTYKDT |
| | | AHTEAMHASMHTNTAVANVGTSISGHESQSSVPADSHTSKATSPM |
| | | GITFAMGDTSVSTSTPAFFETRIQTESTSSLIPGLRDTRTSEEINTVTE |
| | | TSTVLSEVPTTTTTEVSRTEVITSSRTTISGPDHSKMSPYISTETITRLS |
| | | TFPFVTGSTEMAITNQTGPIGTISQATLTLDTSSTASWEGTHSPVTQR |
| | | FPHSEETTTMSRSTKQVSWQSPPSVEETSSPSSPVPLPAITSHSSLYS |
| | | AVSGSSPTSALPVTSLLTSGRRKTIDMLDTHSELVTSSLPSASSFSGEI |
| | | LTSEASTNTETIHFSENTAETNMGTTNSMHKLHSSVSIHSQPSGHTP |
| | | PKVTGSMMEDAIVSTSTPGSPETKNVDRDSTSPLTPELKEDSTALV |
| | | MNSTTESNTVFSSVSLDAATEVSRAEVTYYDPTFMPASAQSTKSPDI |
| | | SPEASSSHSNSPPLTISTHKTIATQTGPSGVTSLGQLTLDTSTIATSAG |
| | | TPSARTQDFVDSETTSVMNNDLNDVLKTSPFSAEEANSLSSQAPLL |
| | | VTTSPSPVTSTLQEHSTSSLVSVTSVPTPTLAKITDMDTNLEPVTRSP |
| | | QNLRNTLATSEATTDTHTMHPSINTAVANVGTTSSPNEFYFTVSPDS |
| | | DPYKATSAVVITSTSGDSIVSTSMPRSSAMKKIESETTFSLIFRLRETS |
| | | TSQKIGSSSDISTVFDKAFTAATTEVSRTELTSSSRTSIQGTEKPTMS |
| | | PDTSTRSVTMLSTFAGLTKSEERTIATQTGPHRATSQGTLTWDTSIT |
| | | TSQAGTHSAMTHGFSQLDLSTLTSRVPEYISGTSPPSVEKTSSSSSLL |
| | | SLPAITSPSPVPTTLPESRPSSPVHLTSLPTSGLVKTTDMLASVASLPP |
| | | NLGSTSHKIPTTSEDIKDTEKMYPSTNIAVTNVGTTTSEKESYSSVPA |
| | | YSEPPKVTSPMVTSFNIRDTIVSTSMPGSSEITRIEMESTFSLAHGLK |
| | | GTSTSQDPIVSTEKSAVLHKLTTGATETSRTEVASSRRTSIPQPDHST |
| | | ESPDISTEVIPSLPISLGITESSNMTHITRTGPPLGSTSQGTFTLDTPTTS |
| | | SRAGTHSMATQEFPHSEMTTVMNKDPEILSWTIPPSIEKTSFSSSLM |
| | | PSPAMTSPPVSSTLPKTIHTTPSPMTSLLTPSLVMTTDTLGTSPEPTTS |
| | | SPPNLSSTSHEILTTDEDTTAIEAMHPSTSTAATNVETTSSGHGSQSS |
| | | VLADSEKTKATAPMDTTSTMGHTTVSTSMSVSSETTKIKRESTYSL |
| | | TPGLRETSISQNASFSTDTSIVLSEVPTGTTAEVSRTEVTSSGRTSIPG |
| | | PSQSTVLPEISTRTMTRLFASPTMTESAEMTIPTQTGPSGSTSQDTLT |
| | | LDTSTTKSQAKTHSTLTQRFPHSEMTTLMSRGPGDMSWQSSPSLEN |
| | | PSSLPSLLSLPATTSPPPISSTLPVTISSSPLPVTSLLTSSPVTTTDMLHT |
| | | SPELVTSSPPKLSHTSDERLTTGKDTTNTEAVHPSTNTAASNVEIPSS |
| | | GHESPSSALADSETSKATSPMFITSTQEDTTVAISTPHFLETSRIQKES |
| | | ISSLSPKLRETGSSVETSSAIETSAVLSEVSIGATTEISRTEVTSSSRTSI |
| | | SGSAESTMLPEISTTRKIIKFPTSPILAESSEMTIKTQTSPPGSTSESTFT |
| | | LDTSTTPSLVITHSTMTQRLPHSEITTLVSRGAGDVPRPSSLPVEETS |
| | | PPSSQLSLSAMISPSPVSSTLPASSHSSSASVTSLLIPGQVKTTEVLD |
| | | ASAEPETSSPPSLSSTSVEILATSEVTTDTEKIHPFSNTAVTKVGTSSS |
| | | GHESPSSVLPDSETTKATSAMGTISIMGDTSVSTLTPALSNTRKIQSE |
| | | PASSLTTRLRETSTSEETSLATEANTVLSKVSTGATTEVSRTEAISFS |
| | | RTSMSGPEQSTMSQDISIGTIPRISASSVLTESAKMTITTQTGPSESTL |
| | | ESTLNLNTATTPSWVETHSIVIQGFPHPEMTTSMGRGPGGVSWPSPP |
| | | FVKETSPPSSPLSLPAVTSPHPVSTTFLAHIPPSPLPVTSLLTSGPATTT |
| | | DILGTSTEPGTSSSSSLSTTSHERLTTYKDTAHTEAVHPSTNTGGTN |
| | | VATTSSGYKSQSSVLADSSPMCTTSTMGDTSVLTSTPAFLETRRIQT |
| | | ELASSLTPGLRESSGSEGTSSGTKMSTVLSKVPTGATTEISKEDVTSI |
| | | PGPAQSTISPDISTRTVSWFSTSPVMTESAEITMNTHTSPLGATTQGT |
| | | STLDTSSTTSLTMTHSTISQGFSHSQMSTLMRRGPEDVSWMSPPLLE |
| | | KTRPSFSLMSSPATTSPSPVSSTLPESISSSPLPVTSLLTSGLAKTTDM |
| | | LHKSSEPVTNSPANLSSTSVEILATSEVTTDTEKTHPSSNRTVTDVG |
| | | TSSSGHESTSFVLADSQTSKVTSPMVITSTMEDTSVSTSTPGFFETSR |
| | | IQTEPTSSLTLGLRKTSSSEGTSLATEMSTVLSGVPTGATAEVSRTEV |
| | | TSSSRTSISGFAQLTVSPETSTETITRLPTSSIMTESAEMMIKTQTDPP |
| | | GSTPESTHTVDISTTPNWVETHSTVTQRFSHSEMTTLVSRSPGDML |
| | | WPSQSSVEETSSASSLLSLPATTSPSPVSSTLVEDFPSASLPVTSLLNP |
| | | GLVITTDRMGISREPGTSSTSNLSSTSHERLTTLEDTVDTEDMQPST |
| | | HTAVTNVRTSISGHESQSSVLSDSETPKATSPMGTTYTMGETSVSIS |
| | | TSDFFETSRIQIEPTSSLTSGLRETSSSERISSATEGSTVLSEVPSGATT |
| | | EVSRTEVISSRGTSMSGPDQFTISPDISTEAITRLSTSPIMTESAESAITI |
| | | ETGSPGATSEGTLTLDTSTTTFWSGTHSTASPGFSHSEMTTLMSRTP |
| | | GDVPWPSLPSVEEASSVSSSLSSPAMTSTSFFSTLPESISSSPHPVTAL |
| | | LTLGPVKTTDMLRTSSEPETSSPPNLSSTSAEILATSEVTKDREKIHP |
| | | SSNTPVVNVGTVIYKHLSPSSVLADLVTTKPTSPMATTSTLQNTSVS |
| | | TSTPAFPETMMTQPTSSLTSGLREISTSQETSSATERSASLSGMPTGA |
| | | TTKVSRTEALSLGRTSTPGPAQSTISPEISTETITRISTPLTTTGSAEMT |
| | | ITPKTGHSGASSQGTFTLDTSSRASWPGTHSAATHRSPHSGMTTPM |
| | | SRQPEDVSWPSRPSVEKTSPPSSLVSLSAVTSPSPLYSTPSESSHSSPL |
| | | RVTSLFTPVMMKTTDMLDTSLEPVTTSPPSMNITSDESLATSKATM |
| | | ETEAIQLSENTAVTQMGTISARQEFYSSYPGLPEPSKVTSPVVTSSTI |
| | | KDIVSTTIPASSEITRIEMESTSTLTPTPRETSTSQEIHSATKPSTVPYK |
| | | ALTSATIEDSMTQVMSSSRGPSPDQSTMSQDISTEVITRLSTSPIKTES |
| | | TEMTITTQTGSPGATSRGTLTLDTSTTFMSGTHSTASQGFSHSQMTA |
| | | LMSRTPGDVPWLSHPSVEEASSASFSLSSPVMTSSSPVSSTLPDSIHS |
| | | SSLPVTSLLTSGLVKTTELLGTSSEPETSSPPNLSSTSAEILAITEVTTD |
| | | TEKLEMTNVVTSGYTHESPSSVLADSVTTKATSSMGITYPTGDTNV |
| | | LTSTPAFSDTSRIQTKSKLSLTPGLMETSISEETSSATEKSTVLSSVPT |
| | | GATTEVSRTEAISSSRTSIPGPAQSTMSSDTSMETITRISTPLTRKEST |
| | | DMAITPKTGPSGATSQGTFTLDSSSTASWPGTHSATTQRFPQSVVTT |
| | | PMSRGPEDVSWPSPLSVEKNSPPSSLVSSSSVTSPSPLYSTPSGSSHSS |
| | | PVPVTSLFTSIMMKATDMLDASLEPETTSAPNMNITSDESLAASKAT |
| | | TETEAIHVFENTAASHVETTSATEELYSSSPGFSEPTKVISPVVTSSSI |
| | | RDNMVSTTMPGSSGITRIEIESMSSLTPGLRETRTSQDITSSTETSTVL |
| | | YKMPSGATPEVSRTEVMPSSRTSIPGPAQSTMSLDISDEVVTRLSTS |
| | | PIMTESAEITITTQTGYSLATSQVTLPLGTSMTFLSGTHSTMSQGLSH |
| | | SEMTNLMSRGPESLSWTSPRFVETTRSSSSLTSLPLTTSLSPVSSTLL |
| | | DSSPSSPLPVTSLILPGLVKTTEVLDTSSEPKTSSSPNLSSTSVEIPATS |
| | | EIMTDTEKIHPSSNTAVAKVRTSSSVHESHSSVLADSETTITIPSMGIT |
| | | SAVDDTTVFTSNPAFSETRRIPTEPTFSLTPGFRETSTSEETTSITETSA |
| | | VLYGVPTSATTEVSMTEIMSSNRIHIPDSDQSTMSPDIITEVITRLSSS |
| | | SMMSESTQMTITTQKSSPGATAQSTLTLATTTAPLARTHSTVPPRFL |
| | | HSEMTTLMSRSPENPSWKSSLFVEKTSSSSSLLSLPVTTSPSVSSTLP |
| | | QSIPSSSFSVTSLLTPGMVKTTDTSTEPGTSLSPNLSGTSVEILAASEV |
| | | TTDTEKIHPSSSMAVTNVGTTSSGHELYSSVSIHSEPSKATYPVGTPS |
| | | SMAETSISTSMPANFETTGFEAEPFSHLTSGFRKTNMSLDTSSVTPT |
| | | NTPSSPGSTHLLQSSKTDFTSSAKTSSPDWPPASQYTEIPVDIITPFNA |
| | | SPSITESTGITSFPESRFTMSVTESTHHLSTDLLPSAETISTGTVMPSLS |
| | | EAMTSFATTGVPRAISGSGSPFSRTESGPGDATLSTIAESLPSSTPVPF |
| | | SSSTFTTTDSSTIPALHEITSSSATPYRVDTSLGTESSTTEGRLVMVST |
| | | LDTSSQPGRTSSSPILDTRMTESVELGTVTSAYQVPSLSTRLTRTDGI |
| | | MEHITKIPNEAAHRGTIRPVKGPQTSTSPASPKGLHTGGTKRMETTT |
| | | TALKTTTTALKTTSRATLTTSVYTPTLGTLTPLNASMQMASTIPTEM |
| | | MITTPYVFPDVPETTSSLATSLGAETSTALPRTTPSVFNRESETTASL |
| | | VSRSGAERSPVIQTLDVSSSEPDTTASWVIHPAETIPTVSKTTPNFFH |
| | | SELDTVSSTATSHGADVSSAIPTNISPSELDALTPLVTISGTDTSTTFP |
| | | TLTKSPHETETRTTWLTHPAETSSTIPRTIPNFSHHESDATPSIATSPG |
| | | AETSSAIPIMTVSPGAEDLVTSQVTSSGTDRNMTIPTLTLSPGEPKTI |
| | | ASLVTHPEAQTSSAIPTSTISPAVSRLVTSMVTSLAAKTSTTNRALTN |
| | | SPGEPATTVSLVTHPAQTSPTVPWTTSIFFHSKSDTTPSMTTSHGAES |
| | | SSAVPTPTVSTEVPGVVTPLVTSSRAVISTTIPILTLSPGEPETTPSMA |
| | | TSHGEEASSAIPTPTVSPGVPGVVTSLVTSSRAVTSTTIPILTFSLGEP |
| | | ETTPSMATSHGTEAGSAVPTVLPEVPGMVTSLVASSRAVTSTTLPT |
| | | LTLSPGEPETTPSMATSHGAEASSTVPTVSPEVPGVVTSLVTSSSQV |
| | | NSTSIPTLILSPGELETTPSMATSHGAEASSAVPTPTVSPGVSGVVTP |
| | | LVTSSRAVTSTTIPILTLSSSEPETTPSMATSHGVEASSAVLTVSPEVP |
| | | GMVTSLVTSSRAVTSTTIPTLTISSDEPETTTSLVTHSEAKMISAIPTL |
| | | AVSPTVQGLVTSLVTSSGSETSAFSNLTVASSQPETIDSWVAHPGTE |
| | | ASSVVPTLTVSTGEPFTNISLVTHPAESSSTLPRTTSRFSHSELDTMPS |
| | | TVTSPEAESSSAISTTISPGIPGVLTSLVTSSGRDISATFPTYPESPHES |
| | | EATASWVTHPAVTSTTVPRTTPNYSHSEPDTTPSIATSPGAEATSDF |
| | | PTITVSPDVPDMVTSQVTSSGTDTSITIPTLILSSGEPETTTSFITYSET |
| | | HTSSAIPTLPVSPGASKMLTSLVISSGTDSTTTFPTLTETPYEPETTAI |
| | | QLIHPAETNTMVPRTTPKFSHSKSDTTLPVAITSPGPEASSAVSTTTIS |
| | | PDMSDLVTSLVPSSGTDTSTTFPTLSETPYEPETTATWLTHPAETSTT |
| | | VSGTIPNFSHRGSDTAPSMVTSPGVDTRSGVPTTTIPPSIPGVVTSQV |
| | | TSSATDTSTAIPTLTPSPGEPETTASSATHPGTQTQFTVPIRTVPSSEP |
| | | DTMASWVTHPPQTSTPVSRTTSSFSHSSPDATPVMATSPRTEASSAV |
| | | LTTISPGAPEMVTSQITSSGAATSTTVPTLTHSPGMPETTALLSTHPR |
| | | TETSKTFPASTVFPQVSETTASLTIRPGAETSTALPTQTTSSLFTLLVT |
| | | GTSRVDLSPTASPGVSAKTAPLSTHPGTETSTMIPTSTLSLQLLETTG |
| | | LLATSSSAETSTSTLTLTVSPAVSGLSSASITTDKPQTVTSWNTETSP |
| | | SVTSVGPPEFSRTVTGTTMTLIPSEMPTPPKTSHGEGVSPTTILRTTM |
| | | VEATNLATTGSSPTVAKTTTTFNTLAGSLFTPLTTPGMSTLASESVT |
| | | SRTSYNHRSWISTTSSYNRRYWTPATSTPVTSTFSPGISTSSIPSSTAA |
| | | TVPFMVPFTLNFTITNLQYEEDMRHPGSRKFNATERELQGLLKPLF |
| | | RNSSLEYLYSQCRLASLRPEKDSSATAVDAICTHRPDPEDLGLDRER |
| | | LYWELSNLTNGIQELGPYTLDRNSLYVNGFTHRSSMPTTSTPGTST |
| | | VDVGTSGTPSSSPSPTTAGPLLMPFTLNFTITNLQYEEDMRRTGSRK |
| | | FNTMESVLQGLLKPLFKNTSVGPLYSGCRLTLLRPEKDGAATGVD |
| | | AICTHRLDPKSPGLNREQLYWELSKLTNDIEELGPYTLDRNSLYVN |
| | | GFTHQSSVSTTSTPGTSTVDLRTSGTPSSLSSPTIMAAGPLLVPFTLN |
| | | FTITNLQYGEDMGHPGSRKFNTTERVLQGLLGPIFKNTSVGPLYSG |
| | | CRLTSLRSEKDGAATGVDAICIHHLDPKSPGLNRERLYWELSQLTN |
| | | GIKELGPYTLDRNSLYVNGFTHRTSVPTSSTPGTSTVDLGTSGTPFS |
| | | LPSPATAGPLLVLFTLNFTITNLKYEEDMHRPGSRKFNTTERVLQTL |
| | | LGPMFKNTSVGLLYSGCRLTLLRSEKDGAATGVDAICTHRLDPKSP |
| | | GVDREQLYWELSQLTNGIKELGPYTLDRNSLYVNGFTHWIPVPTSS |
| | | TPGTSTVDLGSGTPSSLPSPTTAGPLLVPFTLNFTITNLKYEEDMHCP |
| | | GSRKFNTTERVLQSLLQPMFKNTSVGPLYSGCRLTLLRSEKDGAAT |
| | | GVDAICTHRLDPKSPGVDREQLYWELSQLTNGIKELGPYTLDRNSL |
| | | YVNGFTHQTSAPNTSTPGTSTVDLGTSGTPSSLPSPTSAGPLLVPFTL |
| | | NFTITNLQYEEDMHHPGSRKFNTTERVLQGLLGPMFKNTSVGLLYS |
| | | GCRLTLLRPEKNGAATGMDAICSHRLDPKSPGLNREQLYWELSQL |
| | | THGIKELGPYTLDRNSLYVNGFTHRSSVAPTSTPGTSTVDLGTSGTP |
| | | SSLPSPTTAVPLLVPFTLNFTITNLQYGEDMRHPGSRKFNTTERVLQ |
| | | GLLGPLFKNSSVGPLYSGCRLISLRSEKDGAATGVDAICTHHLNPQS |
| | | PGLDREQLYWQLSQMTNGIKELGPYTLDRNSLYVNGFTHRSSGLT |
| | | TSTPWTSTVDLGTSGTPSPVPSPTTTGPLLVPFTLNFTITNLQYEENM |
| | | GHPGSRKFNITESVLQGLLKPLFKSTSVGPLYSGCRLTLLRPEKDGV |
| | | ATRVDAICTHRPDPKIPGLDRQQLYWELSQLTHSITELGPYTLDRDS |
| | | LYVNGFTQRSSVPTTSTPGTFTVQPETSETPSSLPGPTATGPVLLPFT |
| | | LNFTITNLQYEEDMRRPGSRKFNTTERVLQGLLMPLFKNTSVSSLY |
| | | SGCRLTLLRPEKDGAATRVDAVCTHRPDPKSPGLDRERLYWKLSQ |
| | | LTHGITELGPYTLDRHSLYVNGFTHQSSMTTTRTPDTSTMHLATSR |
| | | TPASLSGPMTASPLLVLFTINFTITNLRYEENMHHPGSRKENTTERV |
| | | LQGLLRPVFKNTSVGPLYSGCRLTLLRPKKDGAATKVDAICTYRPD |
| | | PKSPGLDREQLYWELSQLTHSITELGPYTLDRDSLYVNGFTQRSSVP |
| | | TTSIPGTPTVDLGTSGTPVSKPGPSAASPLLVLFTLNFTITNLRYEEN |
| | | MQHPGSRKFNTTERVLQGLLRSLFKSTSVGPLYSGCRLTLLRPEKD |
| | | GTATGVDAICTHHPDPKSPRLDREQLYWELSQLTHNITELGPYALD |
| | | NDSLFVNGFTHRSSVSTTSTPGTPTVYLGASKTPASIFGPSAASHLLI |
| | | LFTLNFTITNLRYEENMWPGSRKFNTTERVLQGLLRPLFKNTSVGP |
| | | LYSGCRLTLLRPEKDGEATGVDAICTHRPDPTGPGLDREQLYLELS |
| | | QLTHSITELGPYTLDRDSLYVNGFTHRSSVPTTSTGVVSEEPFTLNFT |
| | | INNLRYMADMGQPGSLKFNITDNVMQHLLSPLFQRSSLGARYTGC |
| | | RVIALRSVKNGAETRVDLLCTYLQPLSGPGLPIKQVFHELSQQTHGI |
| | | TRIGPYSLDKDSLYLNGYNEPGPDEPPTTPKPATTFLPPLSEATTAM |
| | | GYHLKTLTLNFTISNLQYSPDMGKGSATFNSTEGVLQHLLRPLFQK |
| | | SSMGPFYLGCQLISLRPEKDGAATGVDTTCTYHPDPVGPGLDIQQL |
| | | YWELSQLTHGVTQLGFYVLDRDSLFINGYAPQNLSIRGEYQINFHIV |
| | | NWNLSNPDPTSSEYITLLRDIQDKVTTLYKGSQLHDTFRFCLVTNLT |
| | | MDSVLVTVKALFSSNLDPSLVEQVFLDKTLNASFHWLGSTYQLVDI |
| | | HVTEMESSVYQPTSSSSTQHFYLNFTITNLPYSQDKAQPGTTNYQR |
| | | NKRNIEDALNQLFRNSSIKSYFSDCQVSTFRSVPNRHHTGVDSLCNF |
| | | SPLARRVDRVAIYEEFLRMTRNGTQLQNFTLDRSSVLVDGYSPNRN |
| | | EPLTGNSDLPFWAVILIGLAGLLGVITCLICGVLVTTRRRKKEGEYN |
| | | VQQQCPQYYQSHLDLEDLQ |

The present disclosure may be described in terms of the following non-limiting embodiments:

Embodiment 1: An anti-mucin 16 (MUC16) construct comprising an antibody moiety that immunospecifically recognizes a mucin 16 (MUC16) polypeptide, wherein the antibody moiety comprises: a) (i) a variable heavy (VH) chain comprising a heavy chain complementarity determining region (HC-CDR) 1, an HC-CDR2, and an HC-CDR3 of the heavy chain variable domain of SEQ ID NO: 2; and (ii) a variable light (VL) chain comprising a light chain complementarity determining region (LC-CDR) 1, an LC-CDR2, and an LC-CDR3 of the light chain variable domain of SEQ ID NO: 3; or (b) (i) a variable heavy (VH) chain comprising a heavy chain complementarity determining region (HC-CDR) 1, an HC-CDR2, and an HC-CDR3 of the heavy chain variable domain of SEQ ID NO: 10; and ii) a variable light (VL) chain comprising a light chain complementarity determining region (LC-CDR) 1, an LC-CDR2, and an LC-CDR3 of the light chain variable domain of SEQ ID NO: 11.

Embodiment 2: The anti-mucin 16 (MUC16) construct of Embodiment 1 wherein the antibody moiety comprises: (a) (i) a variable heavy (VH) chain comprising a heavy chain complementarity determining region (HC-CDR) 1 comprising the amino acid sequence of SEQ ID NO: 4; a HC-CDR2 comprising the amino acid sequence of SEQ ID NO: 5; and a HC-CDR3 comprising the amino acid sequence of SEQ ID NO: 6; and (ii) a variable light (VL) chain comprising: a light chain complementarity determining region (LC-CDR) 1 comprising the amino acid sequence of SEQ ID NO: 7; a LC-CDR2 comprising the amino acid sequence of SEQ ID NO: 8; and a LC-CDR3 comprising the amino acid sequence of SEQ ID NO: 9; or (b) (i) a variable heavy (VH) chain comprising a HC-CDR1 comprising the amino acid sequence of SEQ ID NO: 12; a HC-CDR2 comprising the amino acid sequence of SEQ ID NO: 13; and a HC-CDR3 comprising the amino acid sequence of SEQ ID NO: 14; and (ii) a variable light (VL) chain comprising: a LC-CDR1 comprising the amino acid sequence of SEQ ID NO: 15; a LC-CDR2 comprising the amino acid sequence of SEQ ID NO: 16; and a LC-CDR3 comprising the amino acid sequence of SEQ ID NO: 17.

Embodiment 3: The anti-MUC16 construct of Embodiment 1 or Embodiment 2, wherein the antibody moiety immunospecifically binds to the ectodomain of MUC16.

Embodiment 4: The anti-MUC16 construct of any one of Embodiments 1-3, wherein the antibody moiety is a full-length antibody, a Fab, a Fab′, a F(ab′)2, an Fv, or a single chain Fv (scFv).

Embodiment 5: The anti-MUC16 construct of any one of Embodiments 1-4, wherein the MUC16 is a human MUC16.

Embodiment 6: the anti-MUC16 construct of any one of Embodiments 1-5, wherein the VH chain and the VL chain are human VH chain and VL chain.

Embodiment 7: The anti-MUC16 construct of any one of Embodiments 1-6, wherein the antibody moiety immunospecifically binds to a MUC16 c114 polypeptide comprising the amino acid sequence of SEQ ID NO: 25.

Embodiment 8: The anti-MUC16 construct of any one of Embodiments 1-6, wherein the anti-MUC16 construct inhibits in vitro invasion of a tumor cell that expresses MUC16 in a Matrigel invasion assay.

Embodiment 9: The anti-MUC16 construct of Embodiment 8, wherein the tumor cell is an ovarian tumor cell.

Embodiment 10: The anti-MUC16 construct of Embodiment 8 or Embodiment 9, wherein the MUC16 is glycosylated.

Embodiment 11: The anti-MUC16 construct of Embodiment 10, wherein the MUC16 is N-glycosylated at N24 or N30 relative to SEQ ID NO: 25.

Embodiment 12: The anti-MUC16 construct of any one of Embodiments 1-11, wherein the antibody moiety is a monoclonal antibody.

Embodiment 13: The anti-MUC16 construct of any one of Embodiments 1-12, wherein the antibody moiety comprises a VH comprising the amino acid sequence of SEQ ID NO: 2.

Embodiment 14: The anti-MUC16 construct of any one of Embodiments 1-13, wherein the antibody moiety comprises a VL comprising the amino acid sequence of SEQ ID NO: 3.

Embodiment 15; The anti-MUC16 construct of any one of Embodiments 1-12, wherein the antibody moiety comprises a VH comprising the amino acid sequence of SEQ ID NO: 10.

Embodiment 16: The anti-MUC16 construct of any one of Embodiments 1-12 or 15, wherein the antibody moiety comprises a VL comprising the amino acid sequence of SEQ ID NO: 11.

Embodiment 17: The anti-MUC16 construct of any one of Embodiments 1-12, wherein the antibody moiety comprises a VH comprising the amino acid sequence of SEQ ID NO: 2 and a VL comprising the amino acid sequence of SEQ ID NO: 3.

Embodiment 18: The anti-MUC16 construct of any one of Embodiments 1-12, wherein the antibody moiety comprises a VH comprising the amino acid sequence of SEQ ID NO: 10 and a VL comprising the amino acid sequence of SEQ ID NO: 11.

Embodiment 19: The anti-MUC16 construct of any one of Embodiments 1-18, wherein the antibody moiety comprises human-derived heavy and light chain constant regions.

Embodiment 20: The anti-MUC16 construct of Embodiment 19, wherein the heavy chain constant region has an isotype selected from the group consisting of gamma 1, gamma 2, gamma 3, and gamma 4.

Embodiment 21: The anti-MUC16 construct of Embodiment 19 or 20, wherein the light chain constant region has an isotype selected from the group consisting of kappa and lambda.

Embodiment 22: The anti-MUC16 construct of any one of Embodiments 1-21, wherein the antibody moiety is an immunoglobulin comprising two identical heavy chains and two identical light chains.

Embodiment 23: The anti-MUC16 construct of Embodiment 22, wherein the immunoglobulin is an IgG.

Embodiment 24: The anti-MUC16 construct of any one of Embodiments 1-22, wherein the anti-MUC16 construct is monospecific.

Embodiment 25: The anti-MUC16 construct of any one of Embodiments 1-22, wherein the anti-MUC16 construct is multispecific.

Embodiment 26: The anti-MUC16 construct of any one of Embodiments 1-22, wherein the anti-MUC16 construct is bispecific.

Embodiment 27: The anti-MUC16 construct of any one of Embodiments 1-22, wherein the anti-MUC16 construct is a tandem scFv, a diabody (Db), a single chain diabody (scDb), a dual-affinity retargeting (DART) antibody, a F(ab′)2, a dual variable domain (DVD) antibody, a knob-into-hole (KiH) antibody, a dock and lock (DNL) antibody, a chemically cross-linked antibody, a heteromultimeric antibody, or a heteroconjugate antibody.

Embodiment 28: The anti-MUC16 construct of Embodiment 27, wherein the construct is a tandem scFv comprising two scFvs linked by a peptide linker.

Embodiment 29: The anti-MUC16 construct of any one of Embodiments 25-28, wherein the antibody moiety that immunospecifically recognizes MUC16 is a first antibody moiety, and wherein the anti-MUC16 construct further comprises a second antibody moiety that immunospecifically recognizes a second antigen.

Embodiment 30: The anti-MUC16 construct of Embodiment 29, wherein the second antigen is an antigen on the surface of a T cell.

Embodiment 31: The anti-MUC16 construct of Embodiment 30, wherein the second antigen is a CD3.

Embodiment 32: The anti-MUC16 construct of Embodiment 31, wherein the second antigen is selected from the group consisting of CD3γ, CD36, CD38, and CD36.

Embodiment 33: The anti-MUC16 construct of Embodiment 32, wherein the second antigen is CD36.

Embodiment 34: The anti-MUC16 construct of any one of Embodiments 1-19 or 24-26, wherein the anti-MUC16 construct is a chimeric antigen receptor (CAR).

Embodiment 35: The anti-MUC16 construct of Embodiment 34, wherein the CAR comprises a co-stimulatory domain.

Embodiment 36: The anti-MUC16 construct of Embodiment 34 or 35, wherein the CAR comprises a CD3 zeta (C) chain cytoplasmic signaling domain.

Embodiment 37: The anti-MUC16 construct of any one of Embodiments 1-36 further conjugated to a peptide agent, a detection agent, an imaging agent, a therapeutic agent, or a cytotoxic agent.

Embodiment 38; A polypeptide comprising an amino acid sequence of one or more of SEQ ID NOs: 2-17 or an amino acid of the anti-MUC16 construct of any one of Embodiments 1-37.

Embodiment 39: A polynucleotide comprising a nucleic acid sequence encoding one or more polypeptides of Embodiment 38.

Embodiment 40: A vector comprising the polynucleotide of Embodiment 39 operably linked to a promoter.

Embodiment 41: A cell comprising the anti-MUC16 construct of any one of Embodiments 1-37, the polypeptide of Embodiment 38, the polynucleotide of Embodiment 39, or the vector of Embodiment 40.

Embodiment 42: The cell of Embodiment 41, wherein the cell is a mammalian cell.

Embodiment 43: The cell of Embodiment 42, wherein the cell is an immune cell.

Embodiment 44: The cell of Embodiment 43, wherein the cell is a lymphocyte.

Embodiment 45: The cell of Embodiment 44, wherein the cell is a T cell or a B cell.

Embodiment 46: A pharmaceutical composition comprising: a therapeutically effective amount of the anti-MUC16 construct of any one of Embodiments 1-37, the polynucleotide of Embodiment 39, the vector of Embodiment 40, or the cell of any one of Embodiments 41-45; and a pharmaceutically acceptable carrier.

Embodiment 47; A method of treating a MUC16-associated disease or disorder in a patient in need thereof, comprising administering to said patient the pharmaceutical composition of Embodiment 46.

Embodiment 48: The method of Embodiment 47, wherein said MUC16-associated disease or disorder is a cancer.

Embodiment 49: The method of Embodiment 47, wherein said cancer is a cancer of the ovary, lung, pancreas, breast, uterine, fallopian tube, or primary peritoneum.

Embodiment 50; The method of Embodiment 47 or 48, wherein said cancer is a metastatic cancer.

Embodiment 51: The method of any one of Embodiments 47-49, wherein the pharmaceutical composition inhibits metastasis in the patient.

Embodiment 52: The method of any one of Embodiments 47-50, wherein said patient is a human patient.

Embodiment 53: A method of producing an effector cell, comprising genetically modifying a cell with one or more nucleic acids encoding the anti-MUC16 construct of any one of Embodiments 1-37.

Embodiment 54: A method of treatment comprising introducing one or more nucleic acids encoding the anti-MUC16 construct of any one of Embodiments 1-37 into one or more primary cells isolated from a patient and administering cells comprising the one or more nucleic acids to the patient.

Embodiment 55: The method of Embodiment 52, further comprising expanding the cells prior to administering the cells to the patient.

Embodiment 56: The method Embodiment 52 or 53, wherein the primary cells are lymphocytes.

Embodiment 57: The method of Embodiment 54, wherein the primary cells are T cells.

Embodiment 58: The method of any one of Embodiments 47-55, wherein the method further comprises administering a therapeutically effective amount of an additional therapeutic agent to the patient.

Embodiment 59: The method of any of any one of Embodiments 53-58, wherein the anti-MUC16 construct is the anti-MUC16 construct of any one of Embodiments 34-36.

Embodiment 60: A method of detecting MUC16 in a sample, comprising: (a) contacting the sample with the anti-MUC16 construct of any one of Embodiments 1-24 and 37; and (b) detecting the binding, directly or indirectly, between the anti-MUC16 construct and any MUC16 in the sample.

Embodiment 61: The method of Embodiment 60, wherein the anti-MUC16 construct is conjugated to a detectable label.

Embodiment 62: The method of Embodiment 61, wherein the detectable label is a chromogenic, enzymatic, radioisotopic, isotopic, fluorescent, toxic, chemiluminescent, nuclear magnetic resonance contrast agent.

Embodiment 63: The method of Embodiment 61 or 62, wherein the binding between the anti-MUC16 construct and any MUC16 in the sample is detected directly by detecting the detectable label.

Embodiment 64: The method of Embodiment 60, wherein the binding between the anti-MUC16 construct and any MUC16 in the sample is detected indirectly using a secondary antibody.

Embodiment 65; A method of diagnosing an individual suspected of having a MUC16-associated disease or disorder, comprising: a) administering an effective amount of the anti-MUC16 construct of any one of Embodiments 1-24 and 37 to the individual; and b) determining the level of the binding, directly or indirectly, between the anti-MUC16 construct and any MUC16 in the individual, wherein a level of the binding above a threshold level indicates that the individual has the MUC16-associated disease or disorder.

Embodiment 66: A method of diagnosing an individual suspected of having a MUC16-associated disease or disorder, comprising: a) contacting a sample comprising cells derived from the individual with the anti-MUC16 construct of any one of Embodiments 1-24 and 37; and b) determining the number of cells in the sample bound to the anti-MUC16 construct, wherein a value for the number of cells bound to the anti-MUC16 construct above a threshold level indicates that the individual has the MUC16-associated disease or disorder.

Embodiment 67: Use of the anti-MUC16 construct of any one of Embodiments 1-37, the polynucleotide of Embodiment 39, the vector of Embodiment 40, or the cell of any one of Embodiments 41-45 for the treatment of a disease or disorder associated with positive MUC16 expression.

Embodiment 68: Use of the anti-MUC16 construct of any one of Embodiments 1-37, the polynucleotide of Embodiment 39, the vector of Embodiment 40, or the cell of any one of Embodiments 41-45 in the manufacture of a medicament for the treatment of a disease or disorder associated with positive MUC16 expression.

Embodiment 69; Use of the anti-MUC16 construct of any one of Embodiments 1-37, the polynucleotide of Embodiment 39, the vector of Embodiment 40, or the cell of any one of Embodiments 41-45 for the diagnosis of a disease or disorder associated with positive MUC16 expression.

Embodiment 70: The use of any one of Embodiments 62-64, wherein the disease or disorder associated with positive MUC16 expression is a cancer.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

What is claimed is:

1. An anti-mucin 16 (MUC16) construct comprising an antibody moiety that immunospecifically recognizes a mucin 16 (MUC16) polypeptide, wherein the antibody moiety comprises:
(a) (i) a variable heavy (VH) chain comprising a heavy chain complementarity determining region (HC-CDR) 1, an HC-CDR2, and an HC-CDR3 of the heavy chain variable domain of SEQ ID NO: 2; and
(ii) a variable light (VL) chain comprising a light chain complementarity determining region (LC-CDR) 1, an LC-CDR2, and an LC-CDR3 of the light chain variable domain of SEQ ID NO: 3;
or
(b) (i) a variable heavy (VH) chain comprising a heavy chain complementarity determining region (HC-CDR) 1, an HC-CDR2, and an HC-CDR3 of the heavy chain variable domain of SEQ ID NO: 10; and
(ii) a variable light (VL) chain comprising a light chain complementarity determining region (LC-CDR) 1, an LC-CDR2, and an LC-CDR3 of the light chain variable domain of SEQ ID NO: 11.

2. The anti-MUC16 construct of claim 1, wherein the antibody moiety comprises:
(a) (i) a variable heavy (VH) chain comprising a heavy chain complementarity determining region (HC-CDR) 1 comprising the amino acid sequence of SEQ ID NO: 4; a HC-CDR2 comprising the amino acid sequence of SEQ ID NO: 5; and a HC-CDR3 comprising the amino acid sequence of SEQ ID NO: 6; and
(ii) a variable light (VL) chain comprising: a light chain complementarity determining region (LC-CDR) 1 comprising the amino acid sequence of SEQ ID NO: 7; a LC-CDR2 comprising the amino acid sequence of SEQ ID NO: 8; and a LC-CDR3 comprising the amino acid sequence of SEQ ID NO: 9;
or
(b) (i) a variable heavy (VH) chain comprising a HC-CDR1 comprising the amino acid sequence of SEQ ID NO: 12; a HC-CDR2 comprising the amino acid sequence of SEQ ID NO: 13; and a HC-CDR3 comprising the amino acid sequence of SEQ ID NO: 14; and
(ii) a variable light (VL) chain comprising: a LC-CDR1 comprising the amino acid sequence of SEQ ID NO: 15; a LC-CDR2 comprising the amino acid sequence of SEQ ID NO: 16; and a LC-CDR3 comprising the amino acid sequence of SEQ ID NO: 17.

3. The anti-MUC16 construct of claim 1 or claim 2, wherein the antibody moiety immunospecifically binds to the ectodomain of MUC16.

4. The anti-MUC16 construct of any one of claims 1-3, wherein the antibody moiety is a full-length antibody, a Fab, a Fab′, a F(ab′)2, an Fv, or a single chain Fv (scFv).

5. The anti-MUC16 construct of any one of claims 1-4, wherein the VH chain is a human VH chain and/or the VL chain is a human VL chain.

6. The anti-MUC16 construct of any one of claims 1-5, wherein the antibody moiety is a monoclonal antibody.

7. The anti-MUC16 construct of any one of claims 1-6, wherein the antibody moiety comprises: (a) a VH comprising the amino acid sequence of SEQ ID NO: 2 and/or a VL comprising the amino acid sequence of SEQ ID NO: 3; or (b) a VH comprising the amino acid sequence of SEQ ID NO: 10 and/or a VL comprising the amino acid sequence of SEQ ID NO: 11.

8. The anti-MUC16 construct of any one of claims 1-7, wherein the antibody moiety comprises human-derived heavy and light chain constant regions.

9. The anti-MUC16 construct of any one of claims 1-8, wherein the antibody moiety is an immunoglobulin comprising two identical heavy chains and two identical light chains.

10. The anti-MUC16 construct of claim 9, wherein the immunoglobulin is an IgG.

11. The anti-MUC16 construct of any one of claims 1-10, wherein the anti-MUC16 construct is monospecific, bispecific or multispecific.

12. The anti-MUC16 construct of any one of claims 1-11, wherein the anti-MUC16 construct is a tandem scFv, a diabody (Db), a single chain diabody (scDb), a dual-affinity retargeting (DART) antibody, a F(ab′)2, a dual variable domain (DVD) antibody, a knob-into-hole (KiH) antibody, a dock and lock (DNL) antibody, a chemically cross-linked antibody, a heteromultimeric antibody, or a heteroconjugate antibody.

13. The anti-MUC16 construct of claim 11 or 12, wherein the antibody moiety that immunospecifically recognizes MUC16 is a first antibody moiety, and wherein the anti-MUC16 construct further comprises a second antibody moiety that immunospecifically recognizes a second antigen.

14. The anti-MUC16 construct of claim 13, wherein the second antigen is a CD3 antigen.

15. The anti-MUC16 construct of any one of claim 1-8 or 11, wherein the anti-MUC16 construct is a chimeric antigen receptor (CAR).

16. The anti-MUC16 construct of any one of claims 1-15 further conjugated to a peptide agent, a detection agent, an imaging agent, a therapeutic agent, or a cytotoxic agent.

17. A polypeptide comprising an amino acid sequence of one or more of SEQ ID NOs: 2-17 or an amino acid of the anti-MUC16 construct of any one of claims 1-16.

18. A polynucleotide comprising a nucleic acid sequence encoding one or more polypeptides of claim 17.

19. A vector comprising the polynucleotide of claim 18 operably linked to a promoter.

20. A cell comprising the anti-MUC16 construct of any one of claims 1-16, the polypeptide of claim 17, the polynucleotide of claim 18, or the vector of claim 19.

21. A pharmaceutical composition comprising: a therapeutically effective amount of the anti-MUC16 construct of any one of claims 1-16, the polypeptide of claim 17, the polynucleotide of claim 18, or the vector of claim 19; and a pharmaceutically acceptable carrier.

22. A method of treating a MUC16-associated disease or disorder in a patient in need thereof, comprising administering to said patient the pharmaceutical composition of claim 21.

23. The method of claim 22, wherein said MUC16-associated disease or disorder is a cancer.

24. The method of claim 22, wherein said cancer is a cancer of the ovary, lung, pancreas, breast, uterine, fallopian tube, or primary peritoneum.

25. A method of producing an effector cell, comprising genetically modifying a cell with one or more nucleic acids encoding the anti-MUC16 construct of any one of claims 1-16.

26. A method of treatment comprising introducing one or more nucleic acids encoding the anti-MUC16 construct of any one of claims 1-16 into one or more primary cells isolated from a patient and administering cells comprising the one or more nucleic acids to the patient.

27. The method of claim 26, wherein the primary cells are T cells.

28. A method of detecting MUC16 in a sample, comprising: (a) contacting the sample with the anti-MUC16 construct of any one of claim 1-11 or 16; and (b) detecting the binding, directly or indirectly, between the anti-MUC16 construct and any MUC16 in the sample.

29. A method of diagnosing an individual suspected of having a MUC16-associated disease or disorder, comprising:
a) administering an effective amount of the anti-MUC16 construct of any one of claim 1-11 or 16 to the individual; and determining the level of the binding, directly or indirectly, between the anti-MUC16 construct and any MUC16 in the individual, wherein a level of the binding above a threshold level indicates that the individual has the MUC16-associated disease or disorder; or
(b) contacting a sample comprising cells derived from the individual with the anti-MUC16 construct of any one of claim 1-11 or 16; and determining the number of cells in the sample bound to the anti-MUC16 construct, wherein a value for the number of cells bound to the anti-MUC16 construct above a threshold level indicates that the individual has the MUC16-associated disease or disorder.

30. Use of the anti-MUC16 construct of any one of claims 1-16, the polypeptide of claim 17, the polynucleotide of claim 18, or the vector of claim 19, or the cell of claim 20 for the treatment of a disease or disorder associated with positive MUC16 expression, in the manufacture of a medicament for the treatment of a disease or disorder associated with positive MUC16 expression, or for the diagnosis of a disease or disorder associated with positive MUC16 expression.
